(19) United States
(12) Patent Application Publication
(10) Pub. No.: US 2011/0183974 A1
(43) Pub. Date: Jul. 28, 2011

(54) 1,2,4-OXADIAZOLE SUBSTITUTED PIPERIDINE AND PIPERAZINE DERIVATIVES AS SMO ANTAGONISTS

(76) Inventors: Gabriella Dessole, Rome (IT); Philip Jones, Brookline, MA (US); Laura Llauger Bufi, Rome (IT); Ester Muraglia, Rome (IT); Jesus Maria Ontoria Ontoria, Rome (IT); Caterina Torrisi, Rome (IT)

(21) Appl. No.: 13/056,027

(22) PCT Filed: Jul. 27, 2009
(86) PCT No.: PCT/GB2009/050926

§ 371 Date: Apr. 13, 2011

(30) Foreign Application Priority Data
Jul. 28, 2008 (GB) 0813740.8

(51) Int. Cl. A61K 31/5355; C07D 413/14; A61K 31/4709; A61P 35/00; A61K 31/4545; A61K 31/454; A61K 31/496

(52) U.S. Cl. 514/234.5; 546/167; 514/314; 546/194; 514/318; 546/199; 514/322; 544/130; 544/363; 514/253.06; 546/15; 514/278

(57) ABSTRACT

The present invention relates to compounds of formula (I): and pharmaceutically acceptable salts, stereoisomers or tautomers thereof which are inhibitors of the Sonic Hedgehog pathway, in particular Smo antagonists. Thus the compounds of this invention are useful for the treatment of diseases associated with abnormal hedgehog pathway activation, including cancer, for example basal cell carcinoma, medulloblastoma, prostate, pancreatic, breast, colon, bone and small cell lung cancers, and cancers of the upper GI tract.

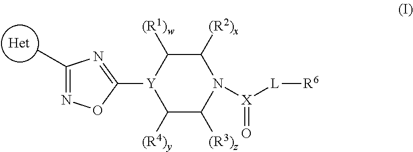

The present invention relates to 1,2,4-oxadiazole substituted piperidine and piperazine derivatives which are inhibitors of the Sonic Hedgehog pathway, in particular Smo antagonists. Thus the compounds of this invention are useful for the treatment of diseases associated with abnormal hedgehog pathway activation, including cancer, for example basal cell carcinoma, medulloblastoma, prostate, pancreatic, breast, colon, bone and small cell lung cancers, and cancers of the upper GI tract.

Hedgehog proteins (Hh) are secreted signaling proteins first discovered in Drosophila. They are highly hydrophobic proteins which after secretion can diffuse and establish gradients in tissues that have a paramount role in the proper development of the embryo. Three Hh homologues with different spatial and temporal distribution patterns have been identified in humans: Sonic hedgehog (SHH), Indian hedgehog (IHH) and Desert hedgehog (DHH).

The Hh signaling cascade is initiated upon binding of Hh to its receptor Patched (Ptch). In the absence of Hh, Ptch inhibits the activity of another membrane spanning protein, Smoothened (Smo) which is a key mediator of Hh signaling. Smo has a structure reminiscent of the G-protein-coupled receptor (GPCR) superfamily, but is not involved in the binding of any Hhs. When Hh is present it binds to Ptch to form an inactive complex, relieving Ptch's inhibition of Smo and activating the Hh response pathway. The Hh signal is then transmitted via a protein complex to the transcription factor cubitus interrupts (Ci) in Drosophila and GLI transcription factors in mammals. In the absence of Hh signaling Ci is cleaved and the amino terminal fragment acts as an inhibitor of Hh target gene transcription. Upon Hh signaling the cleavage of Ci is prevented and Ci becomes an activator of target gene transcription.

Whereas embryonic loss of SHH signaling can result in cyclopia and other developmental defects (Chiang C et al. Nature 383:407-413 (1996)), inappropriate activation of the SHH pathway is believed to lead to increased cell proliferation and tumor formation and is associated with many different types of malignancies, including basal cell carcinoma (BCC), medulloblastoma, pancreatic cancer, small lung cancer, prostate cancer (PC), breast cancer, digestive tract tumors and skin cancer (Kiselyov A S Anti-cancer Agents in Medicinal Chemistry 6:445-449 (2006) and Sidransky D Nature Genet. 14:7-8 (1996)). Thus, the Hh pathway is an important pharmacological target for a variety of conditions.

Aberrant activation of the Hh pathways in cancer are considered to be caused either by mutations in the pathway (ligand independent) or through Hh overexpression (ligand dependent).

Mutations in Ptch 1 have been connected to nevoid basal cell carcinomas syndrome (also called Gorlin syndrome), a condition characterized by a number of development defects and a predisposition for developing numerous basal cell carcinomas (BCC), medulloblastoma, rhabdomyosarcoma and several other neoplasms. Mutations which inactivate Ptch and activate Smo have also been found in sporadic BCC and medulloblastoma, and a number of other sporadic tumors (Reifenberger J et al. Cancer Res. 58:1798-1803 (1998) and Xie J et al. Nature 391:90-92 (1998)).

Plant-derived teratogenic alkaloids cyclopamine and jervine have been proven to cause holoprosencephaly by direct inhibition of SHH signaling (Cooper M K et al. Science 280:1603-1607 (1998) and Incardona J P et al. Development 125:3553-3562 (1998)) by binding to Smo (Chen J K et al. Genes Dev. 16:2743-2748 (2002)). In vitro tests have shown that the teratogen cyclopamine can inhibit the abnormal cell growth of fibroblast cells from Ptch^{-/-} mice, several glioblastoma/glioma cell lines, medulloblastoma cell lines, squamous cell carcinoma cell lines and SCLC cell lines (Bak M et al. Pharmacogenomics 4(4):411-429 (2003)). Cyclopamine has also displayed efficacy in vivo in the models of medulloblastoma (Dahmane N et al. Development 128:5201-5212 (2001) and Berman C M et al. Science 297:1559-1561 (2002)). Synthetic Hh antagonists have been identified in SHH responsive cell models, some targeting Smo (Chen J K et al. Proc. Natl. Acad. Sci. USA 99:14071-14076 (2002), Frank-Kamenetsky M et al. J. Biol. 1:10 (2002) and Williams J A et al. Proc. Natl. Acad. Sci. USA 100:4616-4621 (2003)) and others an unknown target downstream of Smo (Chen J K et al. Proc. Natl. Acad. Sci. USA 99:14071-14076 (2002)).

Reports have shown that Hh overexpression, sometimes accompanied by increased expression of Hh target genes, is detected in a broad spectrum of human tumor biopsies and cell lines, including small cell lung carcinoma, pancreatic adenocarcinoma, oesophageal, stomach and biliary tract cancers, prostate cancer, breast cancer, colon cancer and liver cancer (Rubin L L et al. Nature Reviews Drug Discovery 5:1026-33 (2006)).

The compounds of the present invention are inhibitors of the Hh pathway, in particular Smo antagonists.

The present invention provides a compound of structural formula I:

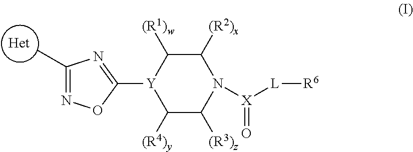

wherein:

each of w, x, y and z is independently 0, 1 or 2;

Y is CH, CR⁵ or N;

L is —(NR⁷)ₐ(O)_{b}(CR⁸R⁹)_{c}(NR⁷)_{d}(C═O)_{f}—;

a is 0 or 1;

b is 0 or 1;

c is 0, 1, 2, 3, 4, 5 or 6;

d is 0 or 1;

f is 0 or 1;

when Y is CH or CR⁵ then each of R¹, R², R³, R⁴ and R⁵ is independently hydroxy, oxo, cyano, halogen, C₁₋₆alkyl, C₂₋₁₀alkenyl, haloC₁₋₆alkyl, hydroxyC₁₋₆alkyl, carboxy, nitro, OR^{a}, CO₂R^{a} or CONR^{a}R^{b};

when Y is N then each of R¹, R², R³ and R⁴ is independently oxo, cyano, C₁₋₆alkyl, C₂₋₁₀alkenyl, haloC₁₋₆alkyl, hydroxyC₁₋₆alkyl, carboxy, CO₂R^{a} or CONR^{a}R^{b};

R⁶ is hydrogen, hydroxy, cyano, halogen, C₁₋₆alkyl, C₂₋₁₀alkenyl, haloC₁₋₆alkyl, hydroxyC₁₋₆alkyl, C₁₋₆alkylcarbonyl, C₁₋₆alkoxy, haloC₁₋₆alkoxy, C₁₋₆alkoxycarbonyl, carboxy, nitro or a ring which is: C₆₋₁₀aryl; C₆₋₁₀aryloxy; C₆₋₁₀arylcarbonyl; C₃₋₁₀cycloalkyl; oxetanyl; azetidinyl; a 5 or 6 membered saturated or partially saturated heterocyclic ring containing one, two or three heteroatoms independently selected from N, O and S; a 5 membered heteroaromatic ring containing 1, 2, 3 or 4 heteroatoms independently selected from N, O and S, not more than one heteroatom of which is O or S; a 6 membered heteroaromatic ring containing one, two or three N atoms; or a 7-15 membered unsaturated, partially saturated or saturated heterocyclic ring containing one, two, three or four heteroatoms independently selected from N, O and S; any of which rings being optionally substituted by one, two or three groups independently selected from (CH₂)ₑ(C═O)_{g}R¹⁰;

e is 0, 1, 2, 3 or 4;

g is 0 or 1;

R⁷ is hydrogen or C₁₋₆alkyl;

each of R⁸ and R⁹ is independently hydrogen, C₁₋₆alkyl, haloC₁₋₆alkyl, NR^{a}R^{b} or a 5 or 6 membered saturated or partially saturated heterocyclic ring containing one, two or three heteroatoms independently selected from N, O and S, which ring is optionally substituted by one, two or three groups independently selected from halogen, C₁₋₆alkyl or haloC₁₋₆alkyl;

Het is pyridin-2-yl or a 7 to 15 membered unsaturated or partially saturated heterocyclic ring containing one, two, three or four heteroatoms independently selected from N, O and S, optionally substituted by one, two or three groups independently selected from R¹¹;

each of R¹⁰ and R¹¹ is independently hydroxy, oxo, oxido, cyano, halogen, C₁₋₆alkyl, C₂₋₁₀alkenyl, haloC₁₋₆alkyl, hydroxyC₁₋₆alkyl, C₁₋₆alkoxyC₁₋₆alkyl, carboxy, nitro, OR^{a}, NR^{a}R^{b}, NR^{a}COR^{b}, NR^{a}S(O)ᵣR^{b}, NR^{a}S(O)ᵣNR^{a}R^{b}, CO₂R^{a}, CONR^{a}R^{b}, S(O)ᵣR^{a}, S(O)ᵣNR^{a}R^{b} or a ring which is: C₃₋₁₀cycloalkyl, C₃₋₁₀cycloalkylC₁₋₆alkyl, C₆₋₁₀aryl, C₆₋₁₀aryloxy, azetidinyl or a 5 or 6 membered saturated or partially saturated heterocyclic ring containing one, two or three heteroatoms independently selected from N, O and S, a 5 membered heteroaromatic ring containing 1, 2, 3 or 4 heteroatoms independently selected from N, O and S, not more than one heteroatom of which is O or S or a 6 membered heteroaromatic ring containing one, two or three N atoms, any of which rings being optionally substituted by one, two or three groups independently selected from hydroxy, oxo, oxido, halogen, C₁₋₆alkyl, haloC₁₋₆alkyl and C₁₋₆alkoxy;

each of R^{a} and R^{b} is independently hydrogen, C₁₋₆alkyl, C₁₋₆alkoxy, C₁₋₆alkylcarbonyl, haloC₁₋₆alkyl, hydroxyC₁₋₆alkyl or C₃₋₁₀cycloalkyl;

r is 0, 1 or 2;

X is C or S═O;

or a pharmaceutically acceptable salt, stereoisomer or tautomer thereof.

In an embodiment:

L is —(NR⁷)ₐ(O)_{b}(CR⁸R⁹)_{c}(NR⁷)_{d}—;

f is 0;

g is 0;

each of R⁸ and R⁹ is independently hydrogen, C₁₋₆alkyl or haloC₁₋₆alkyl;

Het is pyridin-2-yl or a 7 to 15 membered unsaturated heterocyclic ring containing one, two, three or four heteroatoms independently selected from N, O and S, optionally substituted by one, two or three groups independently selected from R¹¹;

each of R¹⁰ and R¹¹ is independently hydroxy, oxo, cyano, halogen, C₁₋₆alkyl, C₂₋₁₀alkenyl, haloC₁₋₆alkyl, hydroxyC₁₋₆alkyl, C₁₋₆alkoxyC₁₋₆alkyl, carboxy, nitro, OR^{a}, NR^{a}R^{b}, NR^{a}COR^{b}, NR^{a}S(O)ᵣR^{b}, NR^{a}S(O)ᵣNR^{a}R^{b}, CO₂R^{a}, CONR^{a}R^{b}, S(O)ᵣR^{a}, S(O)ᵣNR^{a}R^{b} or a ring which is: C₃₋₁₀cycloalkyl, C₆₋₁₀aryl, C₆₋₁₀aryloxy, azetidinyl or a 5 or 6 membered saturated or partially saturated heterocyclic ring containing one, two or three heteroatoms independently selected from N, O and S;

In an embodiment w is 0. In an embodiment w is 1. In another embodiment w is 2.

In an embodiment x is 0. In an embodiment x is 1. In another embodiment x is 2.

In an embodiment y is 0. In an embodiment y is 1. In another embodiment y is 2.

In an embodiment z is 0. In an embodiment z is 1. In another embodiment z is 2.

In an embodiment each of w, x, y and z is independently 0 or 1.

In an embodiment each of w, x, y and z is 0.

In an embodiment Y is CH. In an embodiment Y is CR⁵. In another embodiment Y is N.

In an embodiment a is 0. In another embodiment a is 1.

In an embodiment b is 0. In another embodiment b is 1.

In an embodiment c is 0, 1, 2 or 3. In another embodiment c is 0 or 1. In another embodiment c is 1.

In an embodiment d is 0. In another embodiment d is 1.

In an embodiment f is 0. In another embodiment f is 1.

In an embodiment each of R¹, R², R³, R⁴ and R⁵ is independently oxo, cyano, C₁₋₆alkyl, C₂₋₁₀alkenyl, haloC₁₋₆alkyl, hydroxyC₁₋₆alkyl, carboxy, CO₂R^{a} or CONR^{a}R^{b}.

In an embodiment each of R¹, R², R³, R⁴ and R⁵ is independently hydroxy, halogen or C₁₋₆alkyl.

In an embodiment R¹ is C₁₋₆alkyl.

A particular R¹ group is methyl.

In an embodiment R² is C₁₋₆alkyl.

A particular R² group is methyl.

In an embodiment R⁵ is hydroxy, halogen or C₁₋₆alkyl.

Particular R⁵ groups are hydroxy, fluorine and methyl.

In an embodiment R⁶ is C₁₋₆alkyl or a ring which is: C₃₋₁₀cycloalkyl, C₆₋₁₀aryl, a 5 or 6 membered saturated or partially saturated heterocyclic ring containing one, two or three heteroatoms independently selected from N, O and S, a 5 membered heteroaromatic ring containing 1, 2, 3 or 4 heteroatoms independently selected from N, O and S, not more than one heteroatom of which is O or S, a 6 membered heteroaromatic ring containing one, two or three N atoms or a 8 to 10 membered unsaturated, partially saturated or saturated heterocyclic ring containing one, two, three or four heteroatoms independently selected from N, O and S; any of which rings being optionally substituted by one, two or three groups independently selected from (CH₂)ₑ(C═O)ₑR¹⁰.

In an embodiment R⁶ is C₁₋₆alkyl or a ring which is: C₃₋₁₀cycloalkyl, C₆₋₁₀aryl, a 6 membered saturated heterocyclic ring containing one, two or three heteroatoms independently selected from N, O and S, a 5 membered heteroaromatic ring containing 1, 2, 3 or 4 heteroatoms independently selected from N, O and S, not more than one heteroatom of which is O or S, a 6 membered heteroaromatic ring containing one, two or three N atoms or a 8 to 10 membered unsaturated, partially saturated or saturated heterocyclic ring containing one, two, three or four heteroatoms independently selected from N, O and S; any of which rings being optionally substituted by one, two or three groups independently selected from (CH₂)ₑR¹⁰.

In an embodiment R⁶ is C₁₋₆alkyl, such as methyl haloC₁₋₆alkyl, such as trifluoromethyl or a ring which is: phenyl, cyclohexyl, cycloheptyl, tetrahydrothiopyranyl, cyclopropyl, naphthyl, indolyl, dihydroquinolinyl, furyl, pyridinyl, dihydrobenzodioxinyl, benzofuranyl, cyclobutyl, tetrahydropyranyl, quinolinyl, piperidinyl, dihydroindenyl, adamantyl, benzodioxolyl, cyclopentyl, isoxazolyl, azaazoniaspirononyl, triazolyl, pyrazinyl, azaazoniaspirodecyl, dihydropyridinyl, quinoxalinyl, benzoxazolyl, benzothiazolyl, benzodioxinyl, tetrahydrofuranyl, thiazolyl, tetrahydrothiophenyl, benzimidazolyl, pyrrolidinyl, morpholinyl, dioxanyl, tetrahydroquinolinyl, tetrahydroisoquinolinyl, octahydropyrrolodiazepinyl, octahydropyrrolopyrazinyl, spirooctyl or bicyclopentyl; any of which rings being optionally substituted by one, two or three groups independently selected from (CH₂)ₑ(C═O)_{f}R¹⁰.

In an embodiment R⁶ is C₁₋₆alkyl, such as methyl or a ring which is: phenyl, cyclohexyl, cycloheptyl, tetrahydrothiopyranyl, cyclopropyl, naphthyl, indolyl, dihydroquinolinyl, furyl, pyridinyl, dihydrobenzodioxinyl, benzofuranyl, cyclobutyl, tetrahydropyranyl, quinolinyl, piperidinyl, dihydroindenyl, adamantyl, benzodioxolyl or cyclopentyl; any of which rings being optionally substituted by one, two or three groups independently selected from (CH₂)ₑR¹⁰.

In an embodiment e is 0 or 1. In another embodiment e is 0.

In an embodiment g is 0. In another embodiment g is 1.

In an embodiment R¹⁰ is hydroxy, oxo, oxido, halogen, cyano, C₁₋₆alkyl, haloC₁₋₆alkyl, hydroxyC₁₋₆alkyl, C₁₋₆alkylcarbonyl, C₁₋₆alkoxycarbonyl, NR^{a}R^{b}, CONR^{a}R^{b}, SO₂R^{a}, SO₂NR^{a}R^{b} or a ring which is C₆₋₁₀aryl, C₆₋₁₀aryloxy, a 5 or 6 membered saturated heterocyclic ring containing one, two or three heteroatoms independently selected from N, O and S, a 5 membered heteroaromatic ring containing 1, 2, 3 or 4 heteroatoms independently selected from N, O and S, not more than one heteroatom of which is O or S or a 6 membered heteroaromatic ring containing one, two or three N atoms, any of which rings being optionally substituted by one, two or three groups independently selected from oxido, halogen, C₁₋₆alkyl, haloC₁₋₆alkyl and C₁₋₆alkoxy.

In an embodiment when R¹⁰ is a ring it is unsubstituted or substituted by one or two independently selected groups.

Particular optional substituents on the R¹⁰ ring are selected from oxido, fluoro, methoxy and methyl.

In an embodiment R¹⁰ is halogen, oxo, cyano, haloC₁₋₆alkyl, C₁₋₆alkoxy, C₁₋₆alkyl, C₁₋₆alkylsulfonyl, C₆₋₁₀aryl, C₆₋₁₀aryloxy or a 6 membered saturated heterocyclic ring containing one, two or three heteroatoms independently selected from N, O and S.

Particular R¹⁰ groups are chloro, oxo, phenyl, trifluoromethyl, methoxy, isopropyl, phenoxy, methyl, morpholinyl, cyano, fluoro, methylsulfonyl, piperidinyl, oxido, cyano, acetyl, oxazolyl, hydroxymethyl, hydroxy, pyridinyl, sulfamoyl, dimethylsulfamoyl, dimethylamino, ethyl, butoxycarbonyl, carbamoyl, thiomorpholinyl, dioxidothiomorpholinyl, butoxycarbonylamino, fluoropiperidinyl, difluoropyrrolidinyl, difluoropiperidinyl, acetylamino, pyrrolidinyl, diethylamino, ethylamino, amino, propanylamino, methoxypiperidinyl, cyclohexylamino and methylpiperazinyl.

Specific R¹⁰ groups are chloro, oxo, phenyl, trifluoromethyl, methoxy, isopropyl, phenoxy, methyl, morpholinyl, such as morpholin-4-yl, cyano, fluoro and methylsulfonyl. Further specific R¹⁰ groups are piperidin-1-yl, oxido, cyano, acetyl, 1,3-oxazol-5-yl, hydroxymethyl, hydroxy, pyridin-4-yl, sulfamoyl, dimethylsulfamoyl, dimethylamino, ethyl, tert-butoxycarbonyl, carbamoyl, thiomorpholin-4-yl, 1,1-dioxidothiomorpholin-4-yl, (tert-butoxycarbonyl)amino, 4-fluoropiperidin-1-yl, (3R,4S)-3,4-difluoropyrrolidin-1-yl, 4,4-difluoropiperidin-1-yl, acetylamino, pyrrolidin-1-yl, diethylamino, ethylamino, amino, propanylamino, 4-methoxypiperidin-1-yl, cyclohexylamino and 4-methylpiperazin-1-yl.

In an embodiment L is —NR⁷—, —O—, —(CR⁸R⁹)—, a direct bond, —NR⁷(CR⁸R⁹)—, —NR⁷(CR⁸R⁹)₂NR⁷ or —NR⁷(CR⁸R⁹)₃NR⁷. Further L groups are —NR⁷(CR⁸R⁹)₂—, —NR⁷(CR⁸R⁹)₃ and —NR⁷(CR⁸R⁹)(C═O).

In an embodiment L is —NR⁷—. In another embodiment L is —O—.

In an embodiment L is —NH—, —O—, —CH₂—, a direct bond, —NHCH₂—, —NHCH(CH₃)—, —N(CH₃)—, —NH(CH₂)₂NH—, —NH[C(CH₂CH₃)(CH₃)](CH₂)₂NH— or —N(CH₃)(CH₂)—.

In an embodiment R⁷ is hydrogen or methyl. A further R⁷ group is propyl, for example propan-2-yl.

In an embodiment each of R⁸ and R⁹ is independently hydrogen, C₁₋₆alkyl, di(C₁₋₆alkyl)amino or a 6 membered saturated heterocyclic ring containing one or two heteroatoms independently selected from N and O.

In an embodiment each of R⁸ and R⁹ is independently hydrogen or C₁₋₆alkyl, for example methyl or ethyl.

In an embodiment each of R⁸ and R⁹ is hydrogen.

In an embodiment R⁸ is hydrogen or C₁₋₆alkyl, for example methyl.

In an embodiment R⁹ is hydrogen or C₁₋₆alkyl, for example ethyl. Further R⁹ groups are morpholinyl, for example morpholin-4-yl, piperidinyl, for example piperidin-1-yl and dimethylamino.

Particular R⁶ groups are chlorophenyl, cyclohexyl, cycloheptyl, dichlorophenyl, dioxidotetrahydrothiopyranyl, phenylcyclopropyl, biphenyl, phenyl, bis(trifluoromethyl)phenyl, methoxyphenyl, isopropylphenyl, phenoxyphenyl, (trifluoromethyl)phenyl, methylphenyl, methyl, naphthyl, indolyl, dihydroquinolinyl, methylfuryl, chloropyridinyl, (morpholinylmethyl)phenyl, dihydrobenzodioxinyl, cyanophenyl, benzofuranyl, chlorofluorophenyl, fluoromethylphenyl, pyridinyl, cyclobutyl, tetrahydropyranyl, quinolinyl, (methylsulfonyl)piperidinyl, dihydroindenyl, oxodihydroindenyl, adamantyl, benzodioxolyl and morpholinylcyclopentyl. Further R⁶ groups are difluorocyclohexyl, piperidinylcyclopentyl, dichloropyridinyl, dimethylisoxazolyl, piperidinyl, methylazaazoniaspirononyl, triazolyl, pyrazinyl, oxidopyridinyl, azaazoniaspirodecyl, oxodihydropyridinyl, quinoxalinyl, methylindolyl, chlorocyanophenyl, methylbenzoxazolyl, benzothiazolyl, acetylpiperidinyl, chloro(methylsulfonyl)phenyl, dihydrobenzodioxinyl, cyanopyridinyl, oxazolylphenyl, methyltetrahydrofuranyl, (hydroxymethyl)cyclopentyl, methylthiazolyl, cyclopentyl, hydroxycyclohexyl, pyridinylphenyl, chlorosulfamoylphenyl, benzoxazolyl, (dimethylsulfamoyl)phenyl, methyldioxidotetrahydrothiophenyl, dioxidotetrahydrothiophenyl, methylbenzimidazolyl, tetrahydrothiopyranyl, (methylsulfonyl)phenyl, tetrahydrofuranyl, difluoropiperidinyl, methylcyclohexyl, methyloxopyrrolidinyl, morpholinylcyclohexyl, morpholinylcycloheptyl, piperidinylcyclohexyl, (methylpiperazinyl)cyclohexyl, (dimethylamino)methylpiperidinyl, methylphenylpiperidinyl, fluorophenyl, morpholinyl, trifluoromethyl, ethylpyrrolidinyl, oxopiperidinyl, difluorocyclopentyl, (butoxycarbonyl)piperidinyl, dioxanyl, propanylpyrrolidinyl, methyltetrahydroisoquinolinyl, oxidotetrahydrothiopyranyl, tetrahydroisoquinolinyl, (butoxycarbonyl)tetrahydroisoquinolinyl, carbamoylcyclohexyl, thiomorpholinylcyclopentyl, morpholinyltetrahydrothiophenyl, (dioxidothiomorpholinyl)cyclopentyl, [(butoxycarbonyl)amino]cyclopentyl, methyloxopiperidinyl, (piperidinylcarbonyl)cyclopentyl, difluoro(piperidinyl)cyclohexyl, piperidinyl dihydroindenyl, (fluoropiperidinyl)cyclopentyl, (difluoropyrrolidinyl)cyclopentyl, (difluoropiperidinyl)cyclopentyl, (acetylamino)cyclopentyl, (pyrrolidinylmethyl)cyclopentyl, (dimethylamino)cyclopentyl, (diethylamino)cyclopentyl, pyrrolidinylcyclopentyl, (ethylamino)cyclopentyl, oxido(morpholinyl)tetrahydrothiophenyl, octahydropyrrolodiazepinyl, octahydropyrrolopyrazinyl, ethylpiperidinyl, ethylmorpholinyl, aminocyclopentyl, ethylpyrrolidinyl, phenyltetrahydropyranyl, phenylcyclohexyl, phenylcyclopentyl, spirooctyl, (propanylamino)cyclopentyl, (dimethylamino)tetrahydropyranyl, ethyltetrahydroquinolinyl, piperidinyltetrahydrofuranyl, (methoxypiperidinyl)cyclopentyl, (cyclohexylamino)cyclopentyl, (piperidinylmethyl)cyclopentyl, (methylpiperazinyl)cyclohexyl, propanylpyrrolidinyl, bicyclopentyl, methylcyclobutyl, methylcyclopentyl and methyldioxidotetrahydrothiophenyl.

Specific R⁶ groups are 2-chlorophenyl, cyclohexyl, cycloheptyl, 3,5-dichlorophenyl, 1,1-dioxidotetrahydro-2H-thiopyran-4-yl, (1R,2S)-2-phenylcyclopropyl, 3,4-dichlorophenyl, biphenyl-2-yl, phenyl, 3,5-bis(trifluoromethyl)phenyl, 2-methoxyphenyl, 2,5-dichlorophenyl, 4-chlorophenyl, 2-isopropylphenyl, 4-methoxyphenyl, 3-methoxyphenyl, 4-phenoxyphenyl, 4-(trifluoromethyl)phenyl, 2-methylphenyl, methyl, 2-naphthyl, 2,6-dichlorophenyl, 1H-indol-3-yl, 3,4-dihydroquinolin-1(2H)-yl, 3-methyl-2-furyl, 2,3-dichlorophenyl, 6-chloropyridin-3-yl, 3-(morpholin-4-ylmethyl)phenyl, 2,3-dihydro-1,4-benzodioxin-6-yl, 3-cyanophenyl, 1-benzofuran-6-yl, 4-chloro-2-fluorophenyl, 5-fluoro-2-methylphenyl, 2-(trifluoromethyl)phenyl, pyridin-2-yl, cyclobutyl, 3-chloropyridin-2-yl, tetrahydro-2H-pyran-4-yl, quinolin-8-yl, 1-(methylsulfonyl)piperidin-4-yl, 1-naphthyl, 2,3-dihydro-1H-inden-2-yl, 1-oxo-2,3-dihydroinden-3-yl, 1-adamantyl, 1,3-benzodioxol-5-yl and 1-morpholin-4-ylcyclopentyl. Further specific R⁶ groups are 4,4-difluorocyclohexyl, 1-(piperidin-1-yl)cyclopentyl, 2,6-dichloropyridin-4-yl, 3,5-dimethylisoxazol-4-yl, piperidin-4-yl, 2-methyl-7-aza-2-azoniaspiro[4.4]non-7-yl, 1H-1,2,4-triazol-4-yl, pyrazin-2-yl, 1-oxidopyridin-3-yl, 8-aza-1-azoniaspiro[4.5]dec-8-yl, 2-oxo-1,2-dihydropyridin-3-yl, 4-chloropyridin-3-yl, 2-chloropyridin-3-yl, quinoxalin-6-yl, quinolin-3-yl, 1-methyl-1H-indol-5-yl, 3-chloro-5-cyanophenyl, 2-methyl-1,3-benzoxazol-6-yl, quinolin-6-yl, 1,3-benzothiazol-5yl, 1-acetylpiperidin-4-yl, 2-chloro-5-(methylsulfonyl)phenyl, 2,3-dihydro-1,4-benzodioxin-5-yl, 2-chloro-4-(methylsulfonyl)phenyl, 2-cyanophenyl, 2-chloro-5-cyanophenyl, 6-cyanopyridin-3-yl, 2-(1,3-oxazol-5-yl)phenyl, 2-chloro-4-cyanophenyl, 2-methyltetrahydrofuran-2-yl, 1-benzofuran-5-yl, 1-(hydroxymethyl)cyclopentyl, 4-methyl-1,3-thiazol-2-yl, cyclopentyl, 4-hydroxycyclohexyl, 2-pyridin-4-ylphenyl, 2-chloro-5-sulfamoylphenyl, 1,3-benzoxazol-4-yl, 4-(dimethylsulfamoyl)phenyl, 3-methyl-1,1-dioxidotetrahydrothiophen-3-yl, 1,1-dioxidotetrahydrothiophen-3-yl, 1 methyl-1H-3, 1-benzimidazol-5-yl, 2-chloro-4-sulfamoylphenyl, tetrahydro-2H-thiopyran-4-yl, 4-(methylsulfonyl)phenyl, tetrahydro-2H-pyran-2-yl, tetrahydrofuran-3-yl, 3,3-difluoropiperidin-1-yl, 1-methylcyclohexyl, 1-methyl-2-oxopyrrolidin-3-yl, 1-(morpholin-4-yl)cyclohexyl, 1-(morpholin-4-yl)cycloheptyl, 1-(piperidin-1-yl)cyclohexyl, 1-(4-methylpiperazin-1-yl)cyclohexyl, 4-(dimethylamino)-1-methylpiperidin-4-yl, 1-methyl-4-phenylpiperidin-4-yl, pyridin-3-yl, pyridin-4-yl, 4-fluorophenyl, morpholin-4-yl, trifluoromethyl, 1-ethylpyrrolidin-2-yl, 2-oxopiperidin-3-yl, 3,3-difluorocyclopentyl, tetrahydro-2H-pyran-3-yl, 1-(tert-butoxycarbony)piperidin-4-yl, 1,4-dioxan-2-yl, 1-(propan-2-yl)pyrrolidin-2-yl, 2-methyl-1,2,3-4-tetrahydroisoquinolin-1-yl, 1-oxidotetrahydro-2H-thiopyran-4-yl, 1,2,3,4-tetrahydroisoquinolin-1-yl, 2-(tert-butoxycarbonyl)-1,2,3,4-tetrahydroisoquinolin-1-yl, 4-carbamoylcyclohexyl, 1-methyl-1H-benzimidazol-4-yl, 1-(thiomorpholin-4-yl)cyclopentyl, 3-(morpholin-4-yl)tetrahydrothiophen-3-yl, 1-(1,1-dioxidothiomorpholin-4-yl)cyclopentyl, 1-[(tert-butoxycarbonyl)amino]cyclopentyl, 1-methyl-2-oxopiperidin-3-yl, 1-(piperidin-1-ylcarbonyl)cyclopentyl, 4,4-difluoro-1-(piperidin-1-yl)cyclopentyl, 1-(piperidin-1-yl)-2,3-dihydro-1H-inden-2-yl, 1-(4-fluoropiperidin-1-yl)cyclopentyl, 1-((3R,4S)-3,4-difluoropyrrolidin-1-yl)cyclopentyl, 1-(4,4-difluoropiperidin-1-yl)cyclopentyl, 1-(acetylamino)cyclopentyl, 1-(pyrrolidin-1-ylmethyl)cyclopentyl, 1-(dimethylamino)cyclopentyl, 1-(diethylamino)cyclopentyl, 1-(pyrrolidin-1-yl)cyclopentyl, 1-(ethylamino)cyclopentyl, 4-cyanophenyl, 1-oxido-3-(morpholin-4-yl)tetrahydrothiophen-3-yl, 1,2,3,4-tetrahydroquinolin-2-yl, octahydro-1H-pyrrolo[1,2-a][1,4]diazepin-2-yl, (8aR)-octahydropyrrolo[1,2-a]pyrazin-2-yl, 1-ethylpiperidin-2-yl, (3R)-4-ethylmorpholin-3-yl, 4-ethyl-morpholin-3-yl, (3R)-morpholin-3-yl, (3S)-morpholin-3-yl, (3S)-4-ethylmorpholin-3-yl, 1-aminocyclopentyl, 1-ethylpyrrolidin-2-yl, (2S)-1-ethylpyrrolidin-2-yl, 4-phenyltetrahydro-2H-pyran-4-yl, 1-phenylcyclohexyl, 1-phenylcyclopentyl, spiro[2.5]oct-1-yl, (1R,2R)-2-phenylcyclohexyl, 1-methyl-6-oxopiperidin-3-yl, 1-(propan-2-ylamino)cyclopentyl, 4-(dimethylamino)tetrahydro-2H-pyran-4-yl, 1-ethyl-1,2,3,4-tetrahydroquinolin-2-yl, 1-(piperidin-1-yl)tetrahydrofuran-3-yl, 1-(4-methoxypiperidin-1-yl)cyclopentyl, 1-(cyclohexylamino)cyclopentyl, piperidin-1-yl, 1-(piperidin-1-ylmethyl)cyclopentyl, 1-(4-methylpiperazin-1-yl)cyclohexyl, 1-(propan-2-yl)pyrrolidin-2-yl, bicyclo[1.1.1]pent-1-yl), 1-methylcyclobutyl, 1-methylcyclopentyl and 3-methyl-1,1-dioxidotetrahydrothiophen-3-yl.

In an embodiment Het is pyridin-2-yl or a 8 to 10 membered unsaturated or partially saturated heterocyclic ring containing one, two, three or four heteroatoms independently selected from N, O and S, optionally substituted by one, two or three groups independently selected from R¹¹.

In an embodiment Het is pyridin-2-yl or a 8 to 10 membered unsaturated heterocyclic ring containing one, two, three or four heteroatoms independently selected from N, O and S, optionally substituted by one, two or three groups independently selected from R¹¹.

In an embodiment Het is a 8 to 10 membered unsaturated or partially saturated heterocyclic ring containing one, two, three or four heteroatoms independently selected from N, O and S, optionally substituted by one, two or three groups independently selected from R¹¹.

In an embodiment Het is a 8 to 10 membered unsaturated heterocyclic ring containing one, two, three or four heteroatoms independently selected from N, O and S, optionally substituted by one, two or three groups independently selected from R¹¹.

In an embodiment Het is pyridine-2-yl, quinolinyl, isoquinolinyl, benzimidazolyl, benzothiazolyl, naphthyridinyl, quinoxalinyl, quinazolinyl, tetrahydroquinolinyl, dihydropyranopyridinyl, tetrahydronaphthyridinyl, dihydrothiopyranopyridinyl or benzofuranyl, optionally substituted by one, two or three groups independently selected from R¹¹.

Particular Het groups are quinolinyl, methylpyridin-2-yl, pyridin-2-yl, isoquinolinyl, benzimidazolyl, methylbenzimidazolyl and benzothiazolyl. Further particular Het groups are fluoroquinolinyl, chloroquinolinyl, (fluoro)(methyl)benzimidazolyl, naphthyridinyl, methoxyquinolinyl, quinoxalinyl, quinazolinyl, methylquinolinyl, dimethylpyridinyl, cyanoquinolinyl, tetrahydroquinolinyl, dihydropyranopyridinyl, tetrahydronaphthyridinyl, dioxidodihydrothiopyranopyridinyl, (cyclopropylmethyl)benzimidazolyl, ethylbenzimidazolyl, propanylbenzimidazolyl and benzofuranyl.

In an embodiment when Het is a multicyclic ring then at least one N atom is present in the ring directly adjacent to the 1,2,4-oxadiazole ring.

Specific Het groups are quinolin-2-yl, quinolin-3-yl, 3-methylpyridin-2-yl, pyridin-2-yl, isoquinolin-3-yl, 1H-benzimidazol-2-yl, 1-methyl-1H-benzimidazol-2-yl and 1,3-benzothiazol-2-yl. Further specific Het groups are 5-fluoroquinolin-2-yl, 6-chloroquinolin-2-yl, 6-fluoro-1-methyl-1H-benzimidazol-2-yl, 5-fluoro-1-methyl-1H-benzimidazol-2-yl, 1,6-naphthyridin-2-yl, 6-methoxyquinolin-2-yl, quinoxalin-2-yl, 1,5-naphthyridin-2-yl, 5-chloroquinolin-2-yl, quinazolin-2-yl, 3-methylquinolin-2-yl, 5,6-dimethylpyridin-2-yl, 4-chloroquinolin-2-yl, 5-methoxyquinolin-2-yl, 6-cyanoquinolin-2-yl, 5,6,7,8-tetrahydroquinolin-2-yl, 5-cyanoquinolin-2-yl, 7,8-dihydro-5H-pyrano[4,3-b]pyridin-2-yl, 8-chloroquinolin-2-yl, 7-chloroquinolin-2-yl, 6-fluoroquinolin-2-yl, 7-fluoroquinolin-2-yl, 3-fluoroquinolin-2-yl, 8-fluoroquinolin-2-yl, 5,6,7,8-tetrahydro-1,6-naphthyridin-2-yl, 6,6-dioxido-7,8-dihydro-5H-thiopyranol[4,3-b]pyridin-2-yl, 1-(cyclopropylmethyl)-1H-benzimidazol-2-yl, 1-ethyl-1H-benzimidazol-2-yl, 1-(propan-2-yl)-1H-benzimidazol-2-yl, 1-benzofuran-2-yl, 7-fluoro-1-methyl-1H-benzimidazol-2-yl and 4-fluoro-1-methyl-1H-benzimidazol-2-yl.

In an embodiment Het is not an optionally substituted pyridin-2-yl.

In an embodiment Het is unsubstituted or substituted by one or two independently selected R¹¹ groups.

In an embodiment Het is monosubstituted or unsubstituted.

In an embodiment R¹¹ is oxo, cyano, halogen, C₁₋₆alkyl, C₁₋₆alkoxy or C₃₋₇cycloalkylC₁₋₆alkyl.

In an embodiment R¹¹ is C₁₋₆alkyl, for example methyl.

Particular R¹¹ groups are methyl, fluoro, chloro, methoxy, cyano, oxo, cyclopropylmethyl, ethyl and propanyl.

In an embodiment X is C. In another embodiment X is S═O.

In an embodiment each of R^{a} and R^{b} is independently hydrogen, C₁₋₆alkyl, C₁₋₆alkylcarbonyl, C₁₋₆alkoxycarbonyl or C₃₋₇cycloalkyl.

In an embodiment R^{a} is hydrogen or C₁₋₆alkyl, for example methyl or ethyl.

In an embodiment R^{b} is hydrogen, methyl, ethyl, isopropyl, acetyl, butoxycarbonyl or cyclohexyl.

In an embodiment each R^{a} is independently hydrogen or C₁₋₆alkyl.

In an embodiment each R^{b} is independently hydrogen or C₁₋₆alkyl.

In an embodiment is provided a compound of formula I wherein:

each of w, x, y and z is 0 or 1;

Y is CH, CR⁵ or N;

L is —NR⁷—, —O—, —(CR⁸R⁹)—, a direct bond, —NR⁷(CR⁸R⁹)—, —NR⁷(CR⁸R⁹)₂NR⁷ or —NR⁷(CR⁸R⁹)₃NR⁷;

each of R¹, R², R³, R⁴ and R⁵ is independently hydroxy, halogen or C₁₋₆alkyl;

R⁶ is C₁₋₆alkyl, such as methyl or a ring which is: phenyl, cyclohexyl, cycloheptyl, tetrahydrothiopyranyl, cyclopropyl, naphthyl, indolyl, dihydroquinolinyl, furyl, pyridinyl, dihydrobenzodioxinyl, benzofuranyl, cyclobutyl, tetrahydropyranyl, quinolinyl, piperidinyl, dihydroindenyl, adamantyl, benzodioxolyl or cyclopentyl; any of which rings being optionally substituted by one, two or three groups independently selected from (CH₂)ₑR¹⁰;

e is 0 or 1;

R⁷ is hydrogen or C₁₋₆alkyl;

each of R⁸ and R⁹ is independently hydrogen, C₁₋₆alkyl or haloC₁₋₆alkyl;

Het is a 8 to 10 membered unsaturated heterocyclic ring containing one, two, three or four heteroatoms independently selected from N, O and S, wherein at least one N atom is present in the ring directly adjacent to the 1,2,4-oxadiazole ring, optionally substituted by one, two or three independently selected C₁₋₆alkyl groups;

R¹⁰ is halogen, oxo, cyano, haloC₁₋₆alkyl, C₁₋₆alkoxy, C₁₋₆alkyl, C₁₋₆alkylsulfonyl,

C₆₋₁₀aryl, C₆₋₁₀aryloxy or a 6 membered saturated heterocyclic ring containing one, two or three heteroatoms independently selected from N, O and S;

X is C or S═O.

The present invention also provides a compound of formula II:

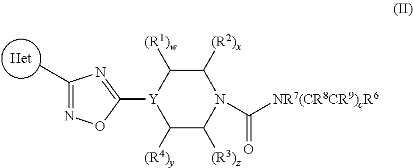

wherein c, w, x, y, z, Y, R¹, R², R³, R⁴, R⁵, R⁶, R⁷, R⁸, R⁹ and Het are as defined above;
or a pharmaceutically acceptable salt, stereoisomer or tautomer thereof.

The present invention also provides a compound of formula III:

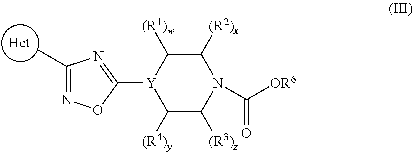

wherein w, x, y, z, Y, R¹, R², R³, R⁴, R⁵, R⁶ and Het are as defined above;
or a pharmaceutically acceptable salt, stereoisomer or tautomer thereof.

The present invention also provides a compound of formula IV:

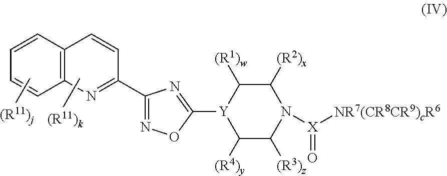

### Wherein:

the sum of j and k is 0, 1, 2 or 3;
c, w, x, y, z, Y, L, R¹, R², R³, R⁴, R⁵, R⁶, R⁷, R⁸, R⁹, R¹¹ and X are as defined above;
or a pharmaceutically acceptable salt, stereoisomer or tautomer thereof.

In an embodiment the sum of j and k is 0, 1 or 2.

The present invention also provides a compound of formula V:

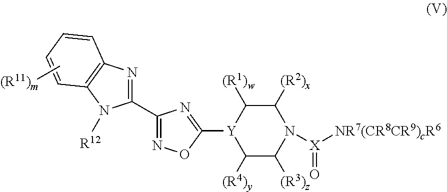

wherein:
m is 0, 1 or 2;
R¹² is hydrogen or R¹¹;
c, w, x, y, z, Y, L, R¹, R², R³, R⁴, R⁵, R⁶, R⁷, R⁸, R⁹, R¹¹ and X are as defined above;
or a pharmaceutically acceptable salt, stereoisomer or tautomer thereof.

In an embodiment R¹² is hydrogen. In another embodiment R¹² is R¹¹.

In an embodiment m is 0 or 1.

The preferred identities with reference to compounds of formulae II, III, IV and V are as defined previously for formula I mutatis mutandis.

For the avoidance of doubt, when w is 0 then the carbon atom to which R¹ can be attached is bonded to two hydrogen atoms; and when w is 1 then this carbon atom is bonded to one hydrogen atom. The same applies when any one or more of x, y and z are 0 or 1, mutatis mutandis.

The present invention also includes within its scope N-oxides of the compounds of formula I above. In general, such N-oxides may be formed on any available nitrogen atom. The N-oxides may be formed by conventional means, such as reacting the compound of formula I with oxone in the presence of wet alumina.

The present invention includes within its scope prodrugs of the compounds of formula I above. In general, such prodrugs will be functional derivatives of the compounds of formula I which are readily convertible in vivo into the required compound of formula I. Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The present invention includes within its scope solvates of the compounds of formula I and salts thereof, for example, hydrates.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention. In addition, the compounds disclosed herein may exist as tautomers and both tautomeric forms are intended to be encompassed by the scope of the invention, even though only one tautomeric structure is depicted.

The compounds may exist in different isomeric forms, all of which are encompassed by the present invention.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

The compounds may exist in a number of different polymorphic forms.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted” should be taken to be equivalent to the phrase “unsubstituted or substituted with one or more substituents” and in such cases the preferred embodiment will have from zero to three substituents. More particularly, there are zero to two substituents. A substituent on a saturated, partially saturated or unsaturated heterocycle can be attached at any substitutable position.

As used herein, “alkyl” is intended to include both branched, straight-chain and cyclic saturated aliphatic hydrocarbon groups having the specified number of carbon atoms. For example, “C₁₋₆alkyl” is defined to include groups having 1, 2, 3, 4, 5 or 6 carbons in a linear, branched or cyclic arrangement. For example, “C₁₋₆alkyl” specifically includes methyl, ethyl, n-propyl, i-propyl, n-butyl, t-butyl, i-butyl, pentyl, hexyl, cyclopropyl, cyclobutyl, cyclopentyl and cyclohexyl and so on. Preferred alkyl groups are methyl and ethyl. The term “cycloalkyl” means a monocyclic, bicyclic or polycyclic saturated aliphatic hydrocarbon group having the specified number of carbon atoms. The multicyclic rings may be fused, bridged or spirolinked. For example, “C₃₋₇cycloalkyl” includes cyclopropyl, methyl-cyclopropyl, 2,2-dimethyl-cyclobutyl, 2-ethyl-cyclopentyl, cyclohexyl, and so on. In an embodiment of the invention the term “cycloalkyl” includes the groups described immediately above and further includes monocyclic unsaturated aliphatic hydrocarbon groups. For example, “cycloalkyl” as defined in this embodiment includes cyclopropyl, methyl-cyclopropyl, 2,2-dimethyl-cyclobutyl, 2-ethyl-cyclopentyl, cyclohexyl, cyclopentenyl, cyclobutenyl, 7,7-dimethylbicyclo[2.2.1]heptyl and so on. Preferred cycloalkyl groups are cyclopropyl, cyclobutyl, cyclopentyl and cyclohexyl.

As used herein, the term “C₂₋₁₀alkenyl” refers to a non-aromatic hydrocarbon radical, straight or branched, containing from 2 to 10, including 2 to 6, carbon atoms and at least one carbon to carbon double bond. Preferably one carbon to carbon double bond is present, and up to four non-aromatic carbon-carbon double bonds may be present. Alkenyl groups include ethenyl, propenyl, butenyl and 2-methylbutenyl. Preferred alkenyl groups include ethenyl and propenyl.

As used herein, the term “C₂₋₁₀alkynyl” refers to a hydrocarbon radical straight or branched, containing from containing from 2 to 10, including 2 to 6 carbon atoms and at least one carbon to carbon triple bond. Up to three carbon-carbon triple bonds may be present. Alkynyl groups include ethynyl, propynyl, butynyl, 3-methylbutynyl and so on. Preferred alkynyl groups include ethynyl and propynyl

“Alkoxy” represents an alkyl group of indicated number of carbon atoms attached through an oxygen bridge. “Alkoxy” therefore encompasses the definitions of alkyl above. Examples of suitable alkoxy groups include methoxy, ethoxy, n-propoxy, i-propoxy, n-butoxy, s-butoxy, t-butoxy, cyclopropyloxy, cyclobutyloxy and cyclopentyloxy. The preferred alkoxy groups are methoxy and ethoxy. The term ‘C₆₋₁₀aryloxy’ can be construed analogously, and an example of this group is phenoxy.

The terms “haloC₁₋₆alkyl” and “haloC₁₋₆alkoxy” mean a C₁₋₆alkyl or C₁₋₆alkoxy group in which one or more (in particular, 1 to 3) hydrogen atoms have been replaced by halogen atoms, especially fluorine or chlorine atoms. Preferred are fluoroC₁₋₆alkyl and fluoroC₁₋₆alkoxy groups, in particular fluoroC₁₋₃alkyl and fluoroC₁₋₃alkoxy groups, for example, CF₃, CHF₂, CH₂F, CH₂CH₂F, CH₂CHF₂, CH₂CF₃, OCF₃, OCHF₂, OCH₂F, OCH₂CH₂F, OCH₂CHF₂ or OCH₂CF₃, and most especially CF₃, OCF₃ and OCHF₂.

As used herein, the term “hydroxyC₁₋₆alkyl” means a C₁₋₆alkyl group in which one or more (in particular, 1 to 3) hydrogen atoms have been replaced by hydroxy groups. Preferred are CH₂OH, CH₂CHOH and CHOHCH₃. The term ‘hydroxyC₂₋₁₀alkenyl’ and ‘hydroxyC₂₋₁₀alkynyl’ can be construed analogously. An example of ‘hydroxyC₂₋₁₀alkynyl’ is (hydroxy)(methyl)butynyl.

As used herein, the term “C₁₋₆alkylcarbonyl” or “C₁₋₆alkoxycarbonyl” denotes a C₁₋₆alkyl or C₁₋₆alkoxy radical, respectively, attached via a carbonyl (C═O) radical. Suitable examples of C₁₋₆alkylcarbonyl groups include methylcarbonyl, ethylcarbonyl, propylcarbonyl, isopropylcarbonyl and tert-butylcarbonyl. Examples of C₁₋₆alkoxycarbonyl include methoxycarbonyl, ethoxycarbonyl, propoxycarbonyl, isopropoxycarbonyl and tert-butoxycarbonyl. The term ‘C₆₋₁₀arylcarbonyl’ can be construed analogously, and an example of this group is benzoyl.

The rings present in the compounds of this invention may be monocyclic or multicyclic, particularly bicyclic. The multicyclic rings may be fused, bridged or spirolinked.

As used herein, “C₆₋₁₀aryl” is intended to mean any stable monocyclic or bicyclic carbon ring of 6 to 10 atoms, wherein at least one ring is aromatic. Examples of such aryl elements include phenyl, naphthyl, tetrahydronaphthyl, indanyl and tetrahydrobenzo[7]annulene. The preferred aryl group is phenyl or naphthyl, especially phenyl.

7-15 membered heterocycles include 7, 8, 9, 10, 11, 12, 13, 14 and 15 membered heterocycles. Similarly, 7-10 membered rings include 7, 8, 9 and 10 membered rings.

Heteroaryl denotes an unsaturated heterocycle ring.

Examples of particular heterocycles of this invention are benzimidazolyl, benzofurandionyl, benzofuranyl, benzofurazanyl, benzopyrazolyl, benzotriazolyl, benzothienyl, benzoxazolyl, benzoxazolonyl, benzothiazolyl, benzothiadiazolyl, benzodioxolyl, benzoxadiazolyl, benzoisoxazolyl, benzoisothiazolyl, chromenyl, chromanyl, isochromanyl, carbazolyl, carbolinyl, cinnolinyl, epoxidyl, furyl, furazanyl, imidazolyl, indolinyl, indolyl, indolizinyl, indolinyl, isoindolinyl, indazolyl, isobenzofuranyl, isoindolyl, isoquinolyl, isothiazolyl, isoxazolyl, naphthpyridinyl, oxadiazolyl, oxazolyl, oxazolinyl, isoxazolinyl, oxetanyl, purinyl, pyranyl, pyrazinyl, pyrazolyl, pyridazinyl, pyridopyridinyl, pyridazinyl, pyridinyl, pyrimidinyl, triazinyl, tetrazinyl, pyrrolyl, quinazolinyl, quinolinyl, quinoxalinyl, quinolizinyl, tetrahydropyranyl, tetrahydrothiopyranyl, tetrahydroisoquinolinyl, tetrazolyl, tetrazolopyridyl, thiadiazolyl, thiazolyl, thienyl, triazolyl, azetidinyl, 1,4-dioxanyl, hexahydroazepinyl, piperazinyl, piperidyl, pyridin-2-onyl, pyrrolidinyl, imidazolinyl, imidazolidinyl, pyrazolinyl, pyrrolinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydroisoquinolinyl, dihydrotetrazolyl, dihydrothiadiazolyl, dihydrothiazolyl, dihydrothienyl, dihydrotriazolyl, dihydroazetidinyl, dihydroisochromenyl, dihydrochromenyl, dihydroimidazolonyl, dihydrotriazolonyl, dihydrobenzodioxinyl, dihydrothiazolopyrimidinyl, dihydroimidazopyrazinyl, methylenedioxybenzoyl, tetrahydrofuranyl, tetrahydrothienyl, tetrahydroquinolinyl, thiazolidinonyl, imidazolonyl, isoindolinonyl, octahydroquinolizinyl, octahydroisoindolyl, imidazopyridinyl, azabicycloheptanyl, chromenonyl, triazolopyrimidinyl, dihydrobenzoxazinyl, thiazolotriazolyl, azoniabicycloheptanyl, azoniabicyclooctanyl, phthalazinyl, naphthyridinyl, pteridinyl, dihydroquinazolinyl, dihydrophthalazinyl, benzisoxazolyl, tetrahydronaphthyridinyl, dibenzo[b,d]furanyl, dihydrobenzothiazolyl, imidazothiazolyl, tetrahydroindazolyl, tetrahydrobenzothienyl, hexahydronaphthyridinyl, tetrahydroimidazopyridinyl, tetrahydroimidazopyrazinyl, pyrrolopyridinyl, diazepanyl, azoniabicyclohexanyl, azoniabicycloheptanyl, azepanyl, octahydropyridopyrazinyl, diazabicycloheptanyl diazoniaspirodecanyl, diazoniaspirononanyl, octahydropyrrolopyrrolyl and tetrahydrotriazolopyrazinyl and N-oxides thereof. Attachment of a heterocyclyl substituent can occur via a carbon atom or via a heteroatom.

Preferred 5 or 6 membered saturated or partially saturated heterocycles are pyrrolidinyl, piperidinyl, piperazinyl, morpholinyl, tetrahydrofuran, thiomorpholinyl, azoniabicyclohexanyl, azoniabicycloheptanyl and tetrahydropyranyl.

Preferred 5 membered heteroaromatic rings are thienyl, thiazolyl, pyrazolyl, isoxazolyl, isothiazolyl, imidazolyl, thiadiazolyl, oxazolyl, oxadiazolyl, triazolyl, tetrazolyl, furyl and pyrrolyl.

Preferred 6 membered heteraromatic rings are pyridinyl, pyrimidinyl, pyridazinyl and pyrazinyl.

Preferred 7-15 membered saturated, partially saturated or unsaturated heterocyclic rings are diazepanyl, azepanyl, tetrahydroquinolinyl, quinolinyl, indolyl, imidazopyridinyl, benzothiazolyl, quinoxalinyl, benzothiadiazolyl, benzoxazolyl, dihydrobenzodioxinyl, benzotriazolyl, benzodioxolyl, dihydroisoindolyl, dihydroindolyl, tetrahydroisoquinolinyl, isoquinolinyl, benzoisothiazolyl, dihydroimidazopyrazinyl, benzothienyl, benzoxadiazolyl, thiazolotriazolyl, dihydrothiazolopyrimidinyl, dihydrobenzoxazinyl, dihydrobenzofuranyl, benzimidazolyl, benzofuranyl, dihydrobenzoxazolyl, dihydroquinazolinyl, dihydrophthalazinyl, indazolyl, benzisoxazolyl, tetrahydronaphthyridinyl, triazolopyrimidinyl, dibenzo[b,d]furanyl, naphthyridinyl, dihydroquinolinyl, dihydroisochromenyl, dihydrochromenyl, dihydrobenzothiazolyl, imidazothiazolyl, tetrahydroindazolyl, tetrahydrobenzothienyl, hexahydronaphthyridinyl, tetrahydroimidazopyridinyl, tetrahydroimidazopyrazinyl, pyrrolopyridinyl, quinazolinyl, indolizinyl, octahydropyridopyrazinyl, diazabicycloheptanyl, diazoniaspirodecanyl, diazoniaspirononanyl, octahydropyrrolopyrrolyl and tetrahydrotriazolopyrazinyl.

As used herein, the term “halogen” refers to fluorine, chlorine, bromine and iodine, of which fluorine and chlorine are preferred.

Particular compounds within the scope of the present invention are the specific compounds named in the representative Examples, and pharmaceutically acceptable salts, free bases, stereoisomers and tautomers thereof.

Included in the instant invention is the free base of compounds of Formula I, as well as the pharmaceutically acceptable salts and stereoisomers thereof. The compounds of the present invention can be protonated at the N atom(s) of an amine and/or N containing heterocycle moiety to form a salt. The term “free base” refers to the amine compounds in non-salt form. The encompassed pharmaceutically acceptable salts not only include the salts exemplified for the specific compounds described herein, but also all the typical pharmaceutically acceptable salts of the free form of compounds of Formula I. The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents.

Thus, pharmaceutically acceptable salts of the compounds of this invention include the conventional non-toxic salts of the compounds of this invention as formed by reacting a basic instant compound with an inorganic, organic acid or polymeric acid. For example, conventional non-toxic salts include those derived from inorganic acids such as hydrochloric, hydrobromic, hydroiodic, sulfuric, sulfurous, sulfamic, phosphoric, phosphorous, nitric and the like, as well as salts prepared from organic acids such as maleic, pamoic, hydroxymaleic, glutamic, salicylic, toluenesulfonic, methanesulfonic, ethane disulfonic, oxalic, aspartic, ethanesulfonic, ethane, disulfonic, trifluoroacetic and the like. Examples of suitable polymeric salts include those derived from the polymeric acids such as tannic acid, carboxymethyl cellulose. Preferably, a pharmaceutically acceptable salt of this invention contains 1 equivalent of a compound of formula (I) and 1, 2 or 3 equivalent of an inorganic or organic acid. More particularly, pharmaceutically acceptable salts of this invention are the trifluoroacetate or the chloride salts. In an embodiment the salt is trifluoroacetate. In another embodiment the salt is chloride.

The preparation of the pharmaceutically acceptable salts described above and other typical pharmaceutically acceptable salts is more fully described by Berg et al (1977) J. Pharm. Sci., ‘Pharmaceutical Salts’, 66:1-19.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

The compounds of this invention may be administered to mammals, preferably humans, either alone or in combination with pharmaceutically acceptable carriers, excipients, diluents, adjuvants, fillers, buffers, stabilisers, preservatives, lubricants, in a pharmaceutical composition, according to standard pharmaceutical practice.

The compounds of this invention may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, (e.g. by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal); and by implant of a depot (e.g. subcutaneously or intramuscularly).

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

The invention also provides pharmaceutical compositions comprising one or more compounds of this invention and a pharmaceutically acceptable carrier. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butanediol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a compound according to this invention is administered into a subject, the selected dosage level will depend on a variety of factors including, but not limited to, the activity of the particular compound, the severity of the individuals symptoms, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician. In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

The present invention provides methods of inhibiting activation of the hedgehog signaling pathway, e.g., to inhibit aberrant growth states resulting from phenotypes such as Ptch loss-of-function, hedgehog gain of-function, smoothened gain-of-function or Gli gain-of-function, comprising contacting the cell with a compound of Formula I, in a sufficient amount to agonize a normal Ptc activity, antagonize a normal hedgehog activity, antagonize smoothened activity, or antagonize Gli activity e.g., to reverse or control the aberrant growth state.

The present invention further provides methods for treating, ameliorating one or more of the symptoms of, and reducing the severity of hyperproliferative disorders, i.e. cancer, as well as other hedgehog pathway mediated disorders or conditions.

Many tumors and proliferative conditions have been shown to depend on the hedgehog pathway. The growth of such cells and survival can be affected by treatment with the compounds of the present invention. For example, small molecule inhibition of the hedgehog pathway has been shown to inhibit the growth of basal cell carcinoma (Williams et al. PNAS 100: 4616-21 (2003)), medulloblastoma (Berman et al. Science 297:1559-61 (2002)), pancreatic cancer, gastrointestinal cancers and esophageal cancer (Berman et al. Nature 425:846-51 (2003) and WO 05/013800), lung cancer (Watkins et al. Nature 422:313-7 (2003)), and prostate cancer (Karhadkar et al. Nature 431: 707-12 (2004)).

In addition, it has been shown that many cancer types have uncontrolled activation of the hedgehog pathway, for example, breast cancer (Kubo et al. Cancer Research 64:6071-4 (2004)), heptacellular cancer (Patil et al. (2005) 96th Annual AACR conference, abstract #2942 and Sicklick et al. (2005) ASCO annual meeting, abstract #9610), hematological malignancies (Watkins and Matsui, unpublished results), basal carcinoma (Bale et al. Human Molec. Genet. B:757-762 (2001), Xie et al. Nature 391: 90-92 (1998)), medulloblastoma (Pietsch et al. Cancer Res. 57: 2085-88 (1997)), and gastric cancer (Ma et al. Carcinogenesis May 19, (2005) (EPub)).

Expression of a dysfunctional mutated patched gene has been reported in sporadic and familial BCCs. Patched gene mutations or deletions have also been found in sporadic medulloblastoma, meningiomas, breast carcinoma, esophageal squamous cell carcinoma and bladder tumors (Oncogene (1998) 17, 1167-1172).

The compounds of the present invention can be used for treating or preventing conditions which can be ameliorated by Smo antagonism. The compounds of the invention are also useful for the manufacture of a medicament for treating or preventing the diseases described herein.

The present invention provides the use of a compound of formula I for the manufacture of a medicament for treating or preventing conditions which can be ameliorated by Smo antagonism.

The present invention also provides a method for the treatment or prevention of conditions which can be ameliorated by Smo antagonism, which method comprises administration to a patient in need thereof of an effective amount of a compound of formula I or a composition comprising a compound of formula I.

The compounds, compositions and methods provided herein are particularly deemed useful for the treatment of cancer. Cancers that may be treated by the compounds, compositions and methods of the invention include, but are not limited to: Cardiac: sarcoma (angio sarcoma, fibrosarcoma, rhabdomyo sarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyo sarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

In an embodiment the compounds of this invention can be used for treating or preventing cancers selected from basal cell carcinoma, medulloblastoma, prostate, pancreatic, breast, colon, small cell lung cancers, sarcoma, lymphomas, leukemia, gastrointestinal cancer, multiple myeloma, glioma and heptacellular. Further cancers that can be treated or prevented by the compounds of the present invention include sporadic and familial basal cell carcinomas, sporadic medulloblastoma, meningiomas, breast carcinoma, esophageal squamous cell carcinoma and bladder cancer.

The present invention also provides the use of a compound of formula I, or a pharmaceutically acceptable salt or solvate thereof for the manufacture of a medicament for the treatment or prevention of cancer.

The present invention also provides a method for the treatment or prevention of cancer, which method comprises administration to a patient in need thereof of an effective amount of a compound of formula I or a composition comprising a compound of formula I.

Inhibition of the hedgehog pathway has been shown to ameliorate the symptoms of psoriasis (Tas, et al., Dermatology 20q:126-131 (2004) and US 2004/0072913).

The present invention provides the use of a compound of formula I for the manufacture of a medicament for the treatment or prevention of psoriasis.

The present invention also provides a method for the treatment or prevention of psoriasis, which method comprises administration to a patient in need thereof of an effective amount of a compound of formula I or a composition comprising a compound of formula I

Hedgehog activation has been shown to stimulate angiogenesis (Pola et al. Nature Medicine 7(6):706-711 (2001) and Nagase et al. Genes to Cells 10(6):595-604 (2005)) and thus compounds which act as hedgehog antagonists may be useful as angiogenesis antagonists.

The present invention provides the use of a compound of formula I for the manufacture of a medicament for the treatment or prevention of angiogenesis.

The present invention also provides a method for the treatment or prevention of angiogenesis, which method comprises administration to a patient in need thereof of an effective amount of a compound of formula I or a composition comprising a compound of formula I

Diseases caused by, supported by or associated with angiogenesis which can be treated or prevented by the compounds of formula I include cancer, ocular neovascular disease, age-related macular degeneration, diabetic retinopathy, retinopathy of prematurity, corneal graft rejection, neovascular glaucoma, retrolental fibroplasia, epidemic keratoconjunctivitis, vitamin A deficiency, contact lens overwear, atopic keratitis, superior limbic keratitis, pterygium keratitis sicca, Sjogren's, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, marginal keratolysis, rheumatoid arthritis, systemic lupus, polyarteritis, trauma, Wegeners sarcoidosis, Scleritis, Stevens Johnson disease, periphigoid radial keratotomy, corneal graph rejection, rheumatoid 15 arthritis, osteoarthritis chronic inflammation (e.g., ulcerative colitis or Crohn's disease), hemangioma, Osler-Weber-Rendu disease, and hereditary hemorrhagic telangiectasia.

In an embodiment the compounds of the present invention are useful for treating and preventing cancers associated with patched loss-of function.

In another embodiment the compounds of the present invention are useful for treating and preventing cancers associated with smoothened gain-of function.

The compounds of formula I are also useful as chemo- and radiosensitizers for cancer treatment. They are useful for the treatment of mammals who have previously undergone or are presently undergoing or will be undergoing treatment for cancer. Such other treatments include chemotherapy, radiation therapy, surgery or immunotherapy, such as cancer vaccines.

The instant compounds are particularly useful in combination with therapeutic, anti-cancer and/or radiotherapeutic agents. Thus, the present invention provides a combination of the presently compounds of formula I with therapeutic, anti-cancer and/or radiotherapeutic agents for simultaneous, separate or sequential administration. The compounds of this invention and the other anticancer agent can act additively or synergistically. A synergistic combination of the present compounds and another anticancer agent might allow the use of lower dosages of one or both of these agents and/or less frequent dosages of one or both of the instant compounds and other anticancer agents and/or to administer the agents less frequently can reduce any toxicity associated with the administration of the agents to a subject without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

The therapeutic agent, anti-cancer agent and/or radiation therapy can be administered according to therapeutic protocols well known in the art. It will be apparent to those skilled in the art that the administration of the therapeutic agent, anti-cancer agent and/or radiation therapy can be varied depending on the disease being treated and the known effects of the anti-cancer agent

and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse affects.

In one embodiment, the compounds of formula I can be administered in combination with one or more agent selected from an anti-inflammatory agent, antihistamine, anti-cancer agent, imununomodulator, therapeutic antibody and a protein kinase inhibitor, e.g., a tyrosine kinase inhibitor.

In another embodiment is provided a combination of a compound of formula I and an anti-cancer agent for simultaneous, separate or sequential administration.

Examples of cancer agents or chemotherapeutic agents for use in combination with the compounds of the present invention can be found in Cancer Principles and Practice of Oncology by V. T. Devita and S. Hellman (editors), 6^{th} edition (Feb. 15, 2001), Lippincott Williams & Wilkins Publishers and WO 2006/061638. A person of ordinary skill in the art would be able to discern which combinations of agents would be useful based on the particular characteristics of the drugs and the cancer involved. Such agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs) and agents that interfere with cell cycle checkpoints. Examples of such agents are provided in WO 2006/061638.

Anticancer agents suitable for use in the combination therapy of the present invention include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-161, and Teniposide [VM-261, etc.), and agents that target topoisomerase I (e.g., Camptothecin and Isirinotecan [CPT-1 11, etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin Dl, etc.), anthracyclines (e.g., Daunorubicin [Daunomycin, and Cerubidine], Doxorubicin [Adrianycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-.alpha., etc.] and interleukins [e.g., IL-2, etc.], etc.); 10) adoptive immunotherapy; 11) hematopoietic growth factors; 12) agents that induce tumor cell differentiation (e.g., alltrans-retinoic acid, etc.); 13) gene therapy techniques; 14) antisense therapy techniques; 15) tumor vaccines; 16) therapies directed against tumor metastases (e.g., Batimistat, etc.); 17) inhibitors of angiogenesis and kinase inhibitors.

In an embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Suitable therapeutic antibodies for use in the combination therapy of the present invention include antibodies directed against the HER2 protein, such as trastuzuinab; antibodies directed against growth factors or growth factor receptors, such as bevacizurnab, which targets vascular endothelial growth factor, and OSI-774, which targets epidermal growth factor; antibodies targeting integrin receptors, such as Vitaxin (also known as MEDI-522), and the like.

In an embodiment is provided a method of treating or preventing basal cell carcinoma, pancreatic cancer, prostate cancer, sarcoma, lymphomas, leukemia, gastrointestinal cancer, multiple myeloma, small cell lung cancer, glioma, breast cancer, heptacellular, or medulloblastoma, which method comprises administration to a patient in need thereof of an effective amount of a compound of formula I in combination with another anti-cancer agent.

In an embodiment is provided a method of treating or preventing psoriasis, which method comprises administration to a patient in need thereof of an effective amount of a compound of formula I in combination with one or more other anti-psoriasis agents including, but not limited to, corticosteroids, tar, calcipotriene, tazarotene, calcineurin inhibitors, ultraviolet irradiation, methotrexate, retinoids, cyclosporine, immunomodulatory drugs, etanercept, alefacept, efalizumab, and infliximab.

The compounds of the formula can be used in combination with radiation therapy. The phrase “radiation therapy” refers to the use of electromagnetic or particulate radiation in the treatment of neoplasia and includes the use of ionizing and non-ionizing radiation.

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In another embodiment, conjunctive therapy with an anti-emesis agent selected from a neurokinin-1 receptor antagonist, a 5HT3 receptor antagonist and a corticosteroid is disclosed for the treatment or prevention of emesis that may result upon administration of the instant compounds.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be useful for treating or preventing cancer in combination with siRNA therapeutics.

A compound of the instant invention may also be useful for treating cancer in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); do farabine (Clolar cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); DROMOSTANOLONE PROPIONATE (DROMOSTANOLONE®); DROMOSTANOLONE PROPIONATE (MASTERONE INJECTION®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); vorinostat (Zolinza®) and zoledronate (Zometa®).

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

The compounds of this invention can be prepared according to the following procedures. All variables within the formulae are defined above.

Abbreviations used in the description of the chemistry and in the Examples that follow are:

DCE: dicholoroethane; DCM: dichloromethane; DMF: dimethylformamide; DMSO: dimethylsulfoxide; EtOH: ethanol; EtOAc: ethyl acetate; MeCN: acetonitrile; MeOH: methanol; THF: tetrahydrofuran; TFA: trifluoroacetic acid; TBAF: tetrabutylammonium fluoride; TBTU: O-benzotriazol-1-yl-N,N,N′,N′-tetramethyluronium tetrafluoroborate; CDI: 1,1′-carbonyldiimidazole; LDA: Lithium diisopropylamide; (Boc)₂O: di-tert-butyl dicarbonate; DIPEA: N,N-diisopropylethylamine; TEA: triethylamine; eq.: equivalent(s); sat. aq.: saturated aqueous; RT: room temperature; min: minutes; h: hour(s); M: molar; atm: atmosphere; NMR: nuclear magnetic resonance; MS: mass spectrometry; ES: electrospray; RP-HPLC: reversed phase high-pressure liquid chromatography; AcOH: acetic acid; DBU: 1,8-diazabicyclo[5.4.0]undec-7-ene; m-CPBA: 3-chloroperoxybenzoic acid; and TBAB: tetrabutylammonium bromide.

Compounds of formula I wherein X is C and L is NH(O)_{b}(CR⁸R⁹)_{c}(NR⁷)_{d} can be prepared by reacting a compound of formula IA with a compound of formula IB;

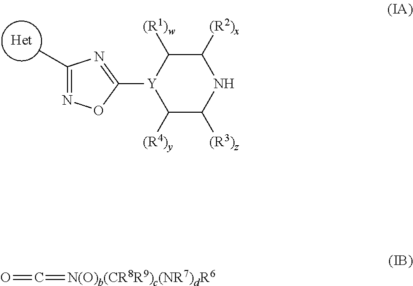

wherein all variables are as defined above. The reaction is generally carried out in the presence of a base such as DIPEA, in solvents such as DCE and DMF at about RT.

Compounds of formula IA wherein Y is CR⁵ can be prepared by reacting a compound of formula IC with a compound of formula ID:

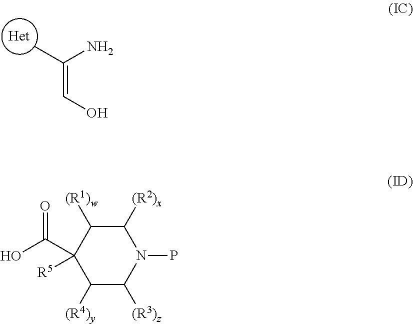

wherein P is a protecting group such as Boc and all other variables are is as defined above. The reaction is generally carried out by firstly reacting with an activating reagent such as TBTU and a base such as DIPEA, in a solvent such as DCM at about RT, followed by cyclizing with a cyclisation agent such as TBAF, in a solvent such as THF at about RT.

The protecting group can be removed according to standard conditions. For example,

when P is Boc it can be removed by the addition of an acid such as HCl in a solvent such as MeOH at about RT.

Compounds of formula IC can be prepared by reacting a compound of formula IE with hydroxylamine:

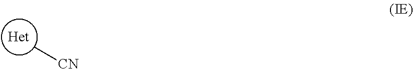

wherein Het is as defined above, generally in the presence of a base such as NaHCO₃, solvents such as EtOH and water at about 80° C.

Alternatively, compounds of formula I wherein X is C and a is 1 can be prepared by converting the compound of formula IA to a chlorocarbamoyl derivative, generally by treatment with triphosgene, in the presence of a base such as DIPEA and in a solvent such as DCM at about −10° C.; followed by reacting with a compound of formula IF:

HNR⁷(O)_{b}(CR⁸R⁹)_{c}(NR⁷)_{d}R⁶ (IF)

wherein all variables are as defined above.

Alternatively, compounds of formula I wherein Y is CR⁵ can be prepared by reacting a compound of formula IC with a compound of formula IG:

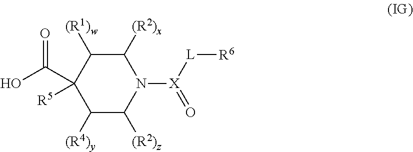

wherein all the variables are as defined above. The reaction is generally carried out under the conditions described above for the reaction between compounds of formulae IC and ID.

Alternatively, compounds of formula I wherein X is C and L is O(CR⁸R⁹)_{c}(NR⁷)_{d} can be prepared by firstly activating the alcohol of formula IH:

HO(CR⁸R⁹)_{c}(NR⁷)_{d}R⁶ (IH)

wherein all the variables are as defined above, generally by using a reagent such as triphosgene, a base such as TEA and in solvents such as DCE and DMF at about RT, followed by addition of a compound of formula IA.

Alternatively, compounds of formula I wherein X is C and L is (CR⁸R⁹)_{c} can be prepared by reacting a compound of formula IA with a compound of formula IJ:

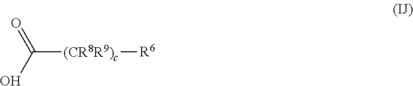

wherein all variables are as defined above. The reaction is generally carried out in the presence of an activating agent such as TBTU, a base such as DIPEA and a solvent such as DMF at about RT.

Compounds of formula I wherein X is S═O and a is 1 can be prepared by reacting a compound of formula IF with a compound of formula IK:

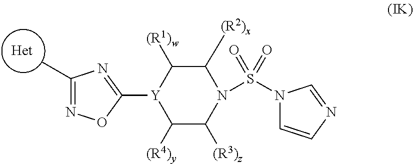

wherein all variables are as defined above. The reaction is generally carried out in the presence of methyl triflate, a base such as MeCN, a solvent such as DCM at about 0° C. to reflux.

Compounds of formula IA wherein Y is N can be prepared by reacting a compound of formula IL with a compound of formula IM:

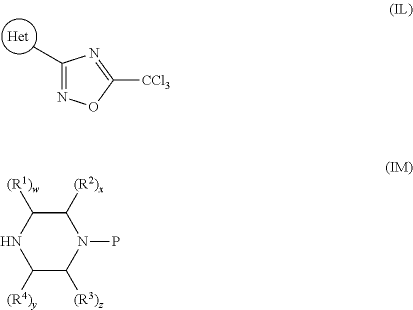

wherein all variables are as defined above. The reaction is generally carried out in a solvent such as THF at about RT.

The protecting group can subsequently be removed according to standard conditions, for example those described above.

Compounds of formula IL can be prepared by reacting a compound of formula IC with a reagent such as trichloroacetyl chloride, generally in the presence of an acid such as trichloroacetic acid at about 90° C.

The compounds of this invention were prepared according to the following schemes. Other methods known in the art can also be used to synthesise the present compounds.

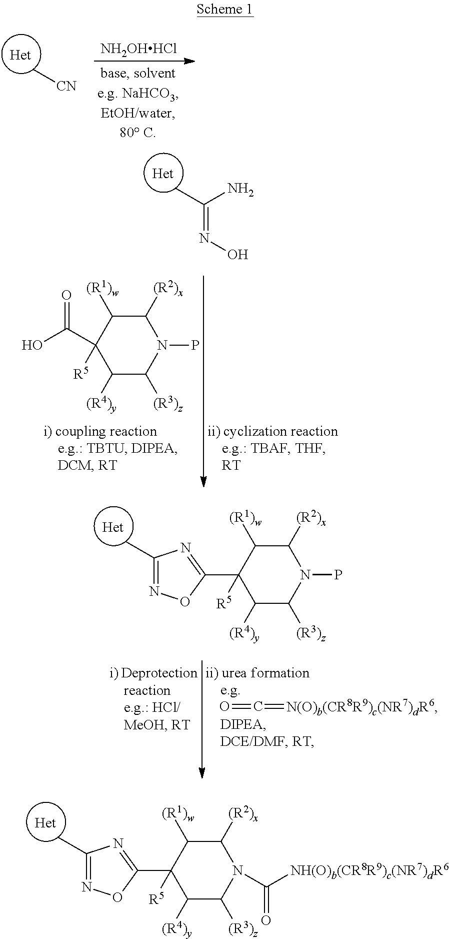

A procedure to synthesize derivatives of compounds of this invention bearing an urea fragment is shown in Scheme 1. Protected 3-heteroaryl-5-piperidin-4-yl-1,2,4-oxadiazol-3-yl analogs can be prepared as described in the literature (i.e.: Williams, J. P. et al. Comb. Chem. & High Thoughput Screening 2000, 3, 43) using a three step synthetic procedure. Heteroaryl amidoximes can be prepared from heteroaryl cyanides by reaction with hydroxylamine in the presence of a base such as NaHCO₃ in a solvent such as EtOH/water at about 80° C. Reaction with protected piperidine-4-carboxylic acid, in the presence of an activating reagent such as TBTU and a base such as DIPEA, followed by treatment with TBAF as described in Gangloff, A. R. et al. Tetrahedron Lett. 2001, 42, 1441 led to the formation of the 1,2,4-oxadiazole intermediate. Other metodologies described in the literature for the formation of the 1,2,4-oxadiazole ring (i.e.: using CDI, as activating reagent, in toluene at 110° C.) can also be applied. Finally, preparation of the ureido derivative was performed by deprotection of the piperidinyl ring by methods known to those skilled in the art (i.e.: if P=Boc, use of acidic media as HCl in MeOH), followed by reaction with an isocyanate in the presence of a base as DIPEA and in a solvent as DCE/DMF.

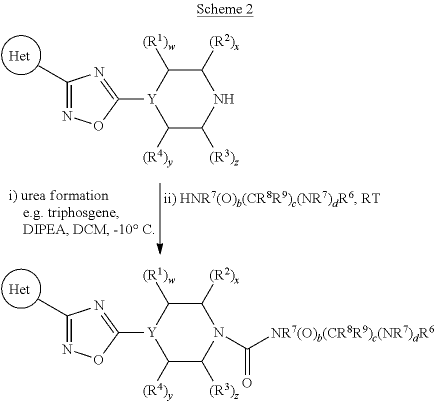

Alternatively, the ureido analog can be prepared by preparation of a chlorocarbamoyl derivative, generally by treating with triphosgene in the presence of a base such as DIPEA and in a solvent such as DCM, followed by addition of the corresponding amine.

The ureido analog can also be synthesized by firstly preparing a chlorocarbamoyl derivative of the amine followed by addition of the compound of formula IA.

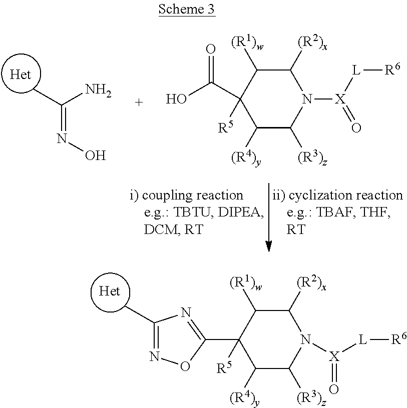

The compounds of this invention can be prepared by reaction of 1-ureidopiperidine-4-carboxylic acids, prepared according to procedures reported in the literature and known to those skilled in the art, with the corresponding heteroarylamidoxime, generally in the presence of an activating reagent as described in Scheme 1, followed by the cyclization reaction.

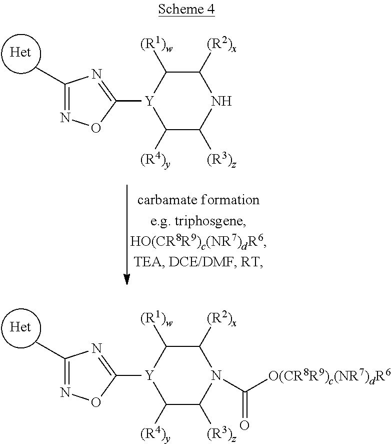

The compounds of this invention which bear a carbamate fragment can be prepared according to the procedure in Scheme 4. Activation of the alcohol was carried out with a reagent such as triphosgene in the presence of a base as TEA, followed by addition of the piperidinyl moiety to produce the desired Smo antagonists.

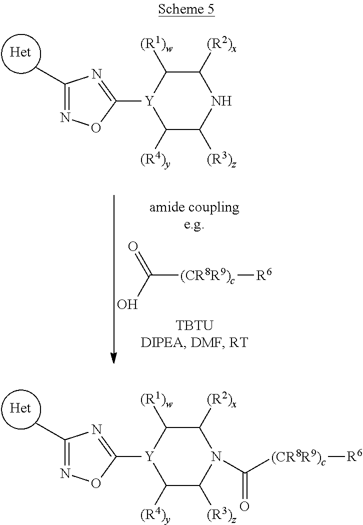

Those compounds of this invention that bears an amide fragment can be prepared as described in Scheme 5. The corresponding carboxylic acid was activated with a reagent such as TBTU in the presence of a base as DIPEA using DMF as a solvent. Then, the piperidinyl moiety was added to afford the desired Smo antagonists. Other methods described in the literature for the formation of amides, for example the use of acid chlorides as acylating reagents, can be applied to obtain the desired amides analogues.

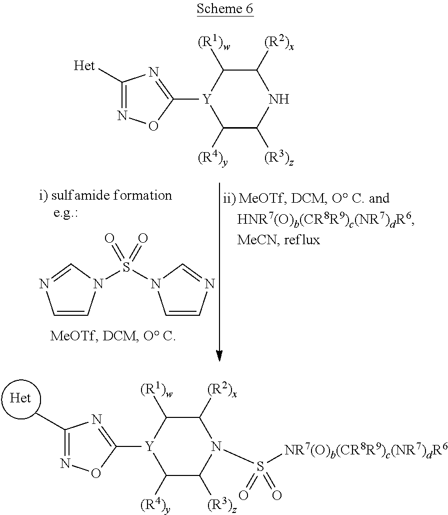

Those compounds of this invention that bears a sulfamide fragment can be prepared following the procedure described in J. Org. Chem. 2003, 68, 115. The piperidinyl or piperazinyl fragment was treated with 1-(1H-imidazol-1-ylsulfonyl)-3-methyl-1H-imidazol-3-ium triflate, previously formed by reaction of N,N′-sulfuryldiimidazole with methyl triflate. This intermediate was treated with methyltriflate and then the corresponding amine was added as described in Scheme 6 to give the desired Smo antagonists.

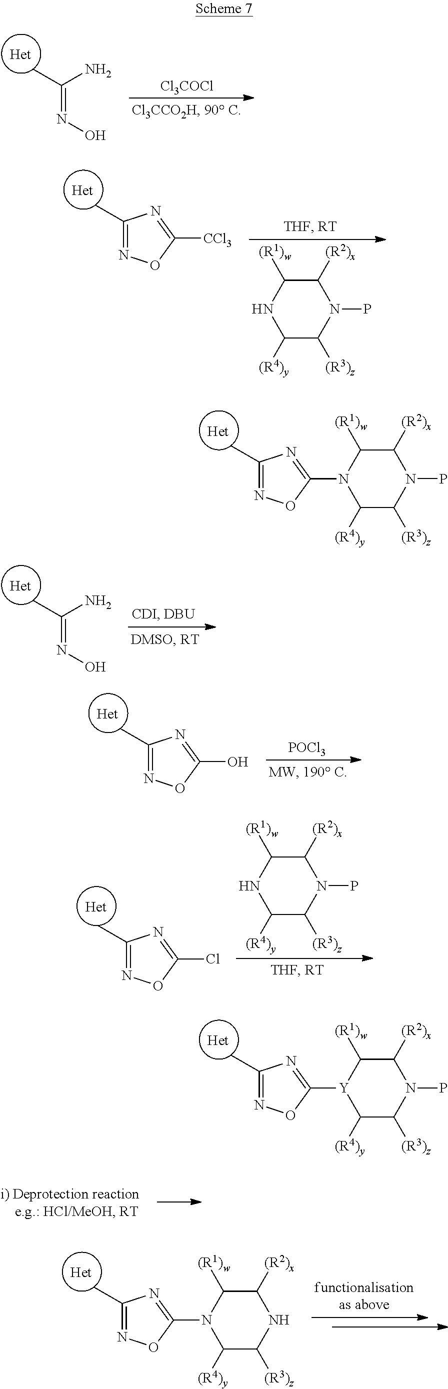

Procedures to synthesize derivatives of those compounds of this invention wherein Y is N are shown in Scheme 7.
Reaction of heteroaryl amidoximes with trichloroacetyl chloride in the presence of trichloroacetic acid at 90° C. led to the formation of 3-heteroaryl-5-(trichloromethyl)-1,2,4-oxadiazole, which by reaction with N′-protected piperazine in a solvent such as THF at RT afforded the protected 1-(3-heteroaryl-1,2,4-oxadiazol-5-yl)piperazine intermediate as described in the literature (e.g. WO 2008/017361).

Alternatively, protected 1-(3-heteroaryl-1,2,4-oxadiazol-5-yl)piperazine intermediate were prepared from heteroaryl amidoximes as described in the scheme below. Reaction of heteroaryl amidoximes with CDI in the presence of a base such as DBU, following the procedure described by Yeh, V. S. C. et al. Biorg. Med. Chem. Lett. 2006, 16, 5414, gave the corresponding 3-heteroaryl-1,2,4-oxadiazol-5-ol. The piperazine moiety was introduced in a two step sequence following the procedure described in WO 2005/110411. Reaction of the 3-heteroaryl-1,2,4-oxadiazol-5-ol intermediate with POCl₃ under heating (i.e.: microwave irradiation at 190° C.) gave the 3-heteroaryl-5-chloro-1,2,4-oxadiazole, which reacted with a monoprotected piperazine derivative to give the protected 1-(3-heteroaryl-1,2,4-oxadiazol-5-yl)piperazine intermediate.

Preparation of the ureido derivative was performed by deprotection of the piperazine ring, followed by functionalisation as described above.

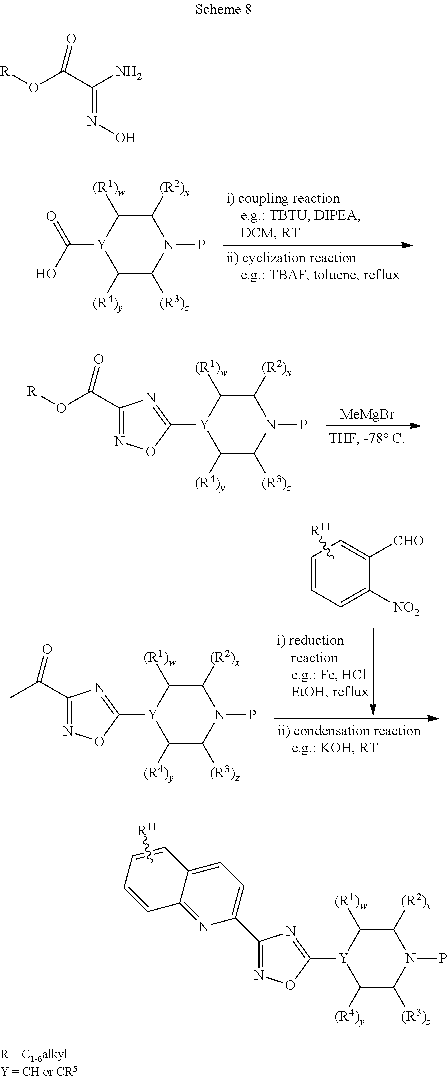

When the Het is a quinoline ring, compounds of formula IA (where Y═CH or CR⁵) can be prepared by an alternative route shown in Scheme 8. Reaction of alkyl (2Z)-amino(hydroxyimino)ethanoate with a protected piperidine-4-carboxylic acid using the same conditions as described in Scheme 1 affords the 3-alkoxycarbonyl-1,2,4-oxadiazole intermediate. Reaction with a methylating reagent such as CH₃MgBr in a solvent such as THF at −78° C. following the procedure described in Jeong, H. et al. Bull. Korean. Che. Soc. 1991, 12, 3, afforded the corresponding methylketone. This intermediate can be converted to protected compound IA using a two step sequence described in Li, A.-H. et al. Org. Biomol. Chem. 2007, 5, 61. Reduction of 2-nitrobenzaldehydes with a metal such as iron under acidic conditions such as 0.1N HCl in a solvent such as EtOH at reflux can be followed by treatment of the resulting aniline with the 3-methylcarbonyl-1,2,4-oxadiazole intermediate in the presence of a base such as KOH to afford the desired compound of formula IA where Y═CH or CR⁵.

Where the synthesis of intermediates and starting materials is not described, these compounds are commercially available or can be made from commercially available compounds by standard methods or by extension of the synthesis above, schemes and Examples herein.

Compounds of formula I may be converted to other compounds of formula I by known methods or by methods described in the Examples herein.

During any of the synthetic sequences described herein it may be necessary and/or desirable to protect sensitive or reactive groups on any of the molecules concerned. This may be achieved by means of conventional protecting groups, such as those described in Protecting Groups in Organic Synthesis, 3rd Edition, Greene, T. W. and Wuts, P. G. M.; Wiley Interscience, 1999 and Kocienski, P. J. Protecting Groups, Thieme, 1994. The protecting groups may be removed at a convenient subsequent stage using methods known from the art. For example, when the Boc (tert-butoxycarbonyl) or benzylcarbonyl protecting group is present, it may be removed by the addition of solvents such as TFA, DCM and/or MeCN at about room temperature. The compound may also be hydrogenated using standard methods, such as treating with a catalyst such as Pd/C, in a solvent such as methanol under a hydrogen atmosphere.

EtOAc in the presence of HCl and 1,4-dioxane may also be added to remove the Boc or benzylcarbonyl protecting group, at about room temperature.

When the compounds of the present invention have chiral centres, the enantiomers may be separated from the racemic mixtures by standard separating methods such as using SFC. The exemplified compounds described herein and tested by the assays described below were found to have an IC₅₀ value of less than 5 uM.

### Shh-Light II Reporter Assay

Assay designed to measure firefly and Renilla luciferase, in the same well.
Prior to assay the Shh-Light II cells (ATCC Catalog No. CRL-2795) were cultured in growth media

### Assay Protocol:

Day −1: seed 60,000 Shh-Light II cells in assay medium 75 uL/well, in presence of DMSO/inhibitor.
Day 0: after overnight incubation at 37° C. 10% CO₂ add 3 uM of Purmorphamine (Calbiochem 540220) in water.
Day 1: After 30 hrs at 37° C. 10% CO₂ of incubation develop the assay, directly to cells in growth medium.

Add 75 μl of DualGlow Luciferase Reagent (Promega, E2940)

Incubate 10 min. in the dark

Read plate at Luminometer: TopCount, by PerkinElmer

Add 75 ul of DualGlow Stop & Glow

Incubate 10 min. in the dark

Read plate at Luminometer: TopCount, by PerkinElmer.
Output is the ratio between FireFly/Renilla counts

### Growth Media:

### For Growth:

DMEM: Dulbecco's Mod Eagle Medium with 0.11G/L Pyr, with Pyridoxine. (GIBCO Cat No: 41966-029). The medium has complemented with 10% FCS (fetal bovine serum), 1% Penicillin-Streptomycin (10 mg/ml) (GIBCO, 15140-114) and 1% L-Glutamine 200 mM(100×) (GIBCO, 3042190) and 0.4 mg/ml of G418 (Roche) and 0.15 mg/ml Zeocyne (Invitrogen R-250-01). Cells cultured at 10% CO₂.
For assay:
DMEM: Dulbecco's Mod Eagle Medium with 0.11G/L Pyr, with Pyridoxine. (GIBCO Cat No: 21063-045), without Phenol Red. The medium has complemented with 2% FCS (fetal bovine serum), 1% Penicillin-Streptomycin (10 mg/ml) (GIBCO, 15140-114) and 1% L-Glutamine 200 mM(100×) (GIBCO, 3042190). Cells cultured at 10% CO₂. DMSO 0.25%.

### SHH Smo Binding Assay

In transfected Cos7 cells we are able to measure the binding of SMO ligand Cyclopamine-bodipy.

### Assay Protocol:

Day −1: Seed 3,500,000 Cos7 cells in Petri dish 10 cm.
Day 0: Transfect cells with Lipofectamine-2000 (Invitrogen) and plasmid pSMO-Myc. After 5 hrs seed the cells in 96 well plate in growth DMEM (10% FCS); 15,000 cells per 100 ul well.
Day 1: 24 hrs after transfection, change the medium with assay DMEM (without Phenol Red 2% FCS) and add compound/DMSO 0.5%. Incubate at 37° C. 5% CO₂.
Day 2: After 16 hrs, add Cyclopamine-Bodipy (Toronto Research Chemical, B674800) at the final concentration of 50 nM. Incubate for 4 hrs at 37° C. 5% CO₂. Then cells are fixed 10 minutes with 3.5% Formaldehyde 100u1/well. Cells are washed 3 times with PBS and nuclei are stained with 1.5 uM Propidium Iodide. Read at Acumen Explorer.

Growth Media:

For growth:
DMEM: GIBCO Dulbecco's Mod Eagle Medium with 0.11G/L Pyr, with Pyridoxine (GIBCO, 41966-029). The medium has complemented with 10% FCS (GIBCO, 10106-169), 1% Penicillin-Streptomycin (10 mg/ml) (GIBCO, 15140-114) and 1% L-Glutamine 200mM(100×) (GIBCO, 3042190). Cells cultured at 5% CO₂
For assay:
DMEM: GIBCO Dulbecco's Mod Eagle Medium with 0.11G/L Pyr, with Pyridoxine (GIBCO, 21063-045) without Phenol Red. The medium has complemented with 2% FCS (GIBCO, 10106-169), 1% Penicillin-Streptomycin (10 mg/ml) (GIBCO, 15140-114) and 1% L-Glutamine 200mM(100×) (GIBCO, 3042190). Cells cultured at 5% CO₂. DMSO 0.5%.

### EXAMPLE 1

### N-(2-Chlorophenyl)-4-(3-quinolin-2-yl-1,2,4-oxadiazol-5-yl)piperidine-1-carboxamide (A2)

### Step 1: 2-(5-Piperidinium-4-yl-1,2,4-oxadiazol-3-yl)quinolinium dichloride (A1)

The title compound was prepared following the procedure described in Gangloff, A. R. et al. Tetrahedron Lett. 2001, 42, 1441, starting from commercially available quinoline-2-carbonitrile and 1-(tert-butoxycarbonyl)piperidine-4-carboxylic acid using a three steps sequence. The title compound was obtained as a yellow powder. ¹H NMR (400 MHz, DMSO-d₆, 300 K) δ 9.14 (1H, bs), 8.65 (1H, d, J=8.7 Hz), 8.22 (2H, d, J=8.7 Hz), 8.15 (1H, d, J=7.3 Hz), 7.92 (1H, ddd, J=8.3, 7.0 and 1.4 Hz), 7.78 (1H, ddd, J=8.0, 6.8 and 1.0 Hz), 3.64-3.59 (1H, m), 3.43-3.40 (2H, m), 3.18-3.09 (2H, m), 2.38-2.34 (2H, m), 2.18-2.08 (2H, m). MS (ES⁺) C₁₆H₁₆N₄O required: 280, found: 281 (M+H)⁺.

### Step 2: N-(2-Chlorophenyl)-4-(3-quinolin-2-yl-1,2,4-oxadiazol-5-yl)piperidine-1-carboxamide (A2)

To a solution of (A1) and DIPEA (1.5 eq.) in DMF/DCE (1:5, 0.3 M) at RT, 1-chloro-2-isocyanatobenzene (2 eq.) was added. The reaction suspension was stirred overnight at RT. The resulting reaction solution was diluted with EtOAc and washed with brine and dried (Na₂SO₄). Evaporation of the solvent under reduced pressure gave a residue which was purified by flash column chromatography on silica using a gradient of EtOAc/Petroleum ether from 30:70 to 100% EtOAc to yield (80%) the title compound as a white powder. ¹H NMR (600 MHz, DMSO-d₆, 300 K) δ 8.65 (1H, d, J=8.6 Hz), 8.32 (1H, s), 8.23 (1H, d, J=8.5 Hz), 8.22 (1H, d, J=8.4 Hz), 8.14 (1H, d, J=7.8 Hz), 7.92 (1H, ddd, J=8.2, 6.9 and 1.2 Hz), 7.78 (1H, ddd, J=8.0, 6.9 and 1.0 Hz), 7.54 (1H, dd, J=8.0 and 1.5 Hz), 7.49 (1H, dd, J=8.0 and 1.3 Hz), 7.33 (1H, ddd, J=7.6 and 1.3 Hz), 7.19 (1H, ddd, J=7.8 and 1.5 Hz), 4.19-4.15 (2H, m), 3.58-3.51 (1H, m), 3.22-3.16 (2H, m), 2.24-2.20 (2H, m), 1.93-1.84 (2H, m). MS (ES⁺) C₂₃H₂₀ClN₅O₂ required: 433, found: 434/436 (M+H)⁺.

### EXAMPLE 2

### 3-(5-{1-[(Cyclohexylamino)carbonyl]piperidin-4-yl}-1,2,4-oxadiazol-3-yl)quinolinium trifluoroacetate (B2)

### Step 1: 3-(5-Piperidinium-4-yl-1,2,4-oxadiazol-3-yl)quinolinium dichloride (B1)

(B1) was prepared following the general procedure reported in Example 1 step 1 starting from commercially available quinoline-3-carbonitrile to yield the title compound as a yellow powder. ¹H NMR (400 MHz, DMSO-d₆, 300 K) δ 9.49 (1H, d, J=2.0 Hz), 9.20-9.00 (3H, m), 8.30 (1H, d, J=7.8 Hz),), 8.17 (1H, d, J=8.3 Hz), 7.96 (1H, ddd, J=8.4, 7.0 and 1.4 Hz), 7.79 (1H, ddd, J=8.0, 6.7 and 0.9 Hz), 3.65-3.59 (1H, m), 3.43-3.40 (2H, m), 3.18-3.09 (2H, m), 2.38-2.33 (2H, m), 2.18-2.08 (2H, m). MS (ES⁺) C₁₆H₁₆N₄O required: 280, found: 281 (M+H)⁺.

### Step 2: 3-(5-{1-[(Cyclohexylamino)carbonyl]piperidin-4-yl}-1,2,4-oxadiazol-3-yl)quinolinium trifluoroacetate (B2)

(B2) was prepared following the general procedure reported in Example 1 step 2, using isocyanatocyclohexane (1.5 eq.). After stirring overnight at RT, the reaction solution was concentrated and the crude was dissolved in EtOAc. The resulting white precipitate was filtered off and the filtrated solution was evaporated to dryness giving a crude that was purified by preparative RP-HPLC, using water (+0.1% TFA) and MeCN (+0.1% TFA) as eluents (column: C18). The desired fractions were lyophilized to yield (77%) the title compound as a white powder. ¹H NMR (400 MHz, DMSO-d₆, 300 K) δ 9.48 (1H, s), 9.08 (1H, s), 8.28 (1H, d, J=8.1 Hz), 8.16 (1H, d, J=8.4 Hz), 8.14 (1H, d, J=7.8 Hz), 7.93 (1H, t, J=7.4 Hz), 7.77 (1H, t, J=7.4 Hz), 6.25 (1H, bs), 4.05-4.02 (2H, m), 3.45-3.43 (2H, m), 2.98-2.93 (2H, m), 2.12-2.10 (2H, m), 1.79-1.71 (6H, m), 1.32-1.04 (6H, m). MS (ES⁺) C₂₃H₂₇N₅O₂ required: 405, found: 406 (M+H)⁺.

### EXAMPLE 3

### N-Cyclohexyl-4-[3-(3-methylpyridin-2-yl)-1,2,4-oxadiazol-5-yl]piperidine-1-carboxamide (C2)

### Step 1: 3-Methyl-2-(5-piperidinium-4-yl-1,2,4-oxadiazol-3-yl)pyridinium dichloride (C1)

(C1) was prepared following the general procedure reported in Example 1 step 1 starting from commercially available 2-cyano-3-methylpyridine to yield the title compound as a pale pink powder. ¹H NMR (400 MHz, DMSO-d₆, 300 K) δ 8.6 (1H, dd, J=4.6 and 0.9 Hz), 7.88 (1H, d, J=7.7 Hz), 7.53 (1H, d, J=7.7 Hz), 7.52 (1H, d, J=7.7 Hz), 3.27-3.23 (1H, m), 3.04-3.01 (2H, m), 2.68-2.63 (2H, m), 2.51 (3H, s), 2.04-2.02 (2H, m), 1.71 (2H, ddt, J=11.3 and 3.8 Hz). MS (ES⁺) C₁₃H₁₆N₄O required: 244, found: 245 (M+H)⁺.

### Step 2: N-Cyclohexyl-4-[3-(3-methylpyridin-2-yl)-1,2,4-oxadiazol-5-yl]piperidine-1-carboxamide (C2)

(C2) was prepared following the general procedure reported in Example 2 step 2. The corresponding crude was purified by preparative RP-HPLC, using water and MeCN as eluents (column: C18). The desired fractions were lyophilized to yield (65%) the title compound as a white powder. ¹H NMR (300 MHz, DMSO-d₆, 300 K) δ 8.61 (1H, d, J=4.2 Hz), 7.88 (1H, d, J=7.9 Hz), 7.55-7.51 (1H, m), 7.52 (1H, t, J=7.6 Hz), 6.22 (1H, d, J=7.6 Hz), 4.02-3.97 (2H, m), 3.46-3.37 (2H, m), 2.98-2.91 (2H, m), 2.51 (3H, s), 2.11-2.05 (2H, m), 1.80-1.58 (7H, m), 1.29-1.08 (5H, m). MS (ES⁺) C₂₀H₂₇N₅O₂ required: 369, found: 370 (M+H)⁺.

### EXAMPLE 4

N-(2-chlorophenol)-3-methyl-4-(3-quinolin-2-yl-1,2,4-oxadiazol-5-yl)piperidine-1-carboxamide (D6)

### Step 1: cis-4-carboxy-3-methylpiperidinium chloride (D1)

To a solution of commercially available 3-methylisonicotinic acid in MeOH (0.5 M), HCl (1.25 M solution in MeOH, 1 eq.) and PtO₂ (20% w/w) were added. The reaction mixture was stirred under hydrogen atm (50 psi) overnight in a Parr apparatus. Then, the catalyst was filtered off and the filtrate was evaporated to dryness yielding quantitatively the title compound as a white foam. The compound is a 4:1 mixture of cis/trans diastereoisomers. ¹H NMR (400 MHz, DMSO-d₆, 300 K) δ 12.50 (1H, brs), 9.19 (1H, s), 8.77 (1H, s), 3.15-3.06 (1H, m), 3.05-2.99 (2H, m), 2.97-2.87 (1H, m), 2.74-2.65 (1H, m), 2.32-2.22 (1H, m), 1.90-1.77 (2H, m), 0.96 (3H, d, J=7.2 Hz). MS (ES⁺) C₇H₁₃NO₂ requires: 143, found: 144 (M+H)⁺.

### Step 2: cis-1-(tert-butoxycarbonyl)-3-methylpiperidine-4-carboxylic acid (D2)

A solution of (D1), (Boc)₂O (1.5 eq.) and NaOH (2.5 eq.) in water (0.2 M) was stirred at RT overnight. The reaction mixture was extracted with DCM, and the organic phase was washed with 1N HCl and brine and dried (Na₂SO₄). Evaporation of the solvent under reduced pressure yielded (90%) the title compound as a white solid. ¹H NMR (400 MHz, DMSO-d₆, 300 K) δ 12.22 (1H, bs), 3.96-3.76 (1H, m), 3.74-3.65 (1H, m), 3.08-2.92 (1H, m), 2.60-2.52 (1H, m), 2.19-2.07 (1H, m), 1.62-1.48 (2H, m), 1.38 (9H, s), 0.80 (3H, d, J=6.9 Hz). MS (ES⁺) C₁₂H₂₁NO₄ requires: 243, found: 244 (M+H)⁺.

### Step 3: 1-(tert-butoxycarbonyl)-3-methylpiperidine-4-carboxylic acid (D3)

A solution of (D2) in dry THF (2 M) was added dropwise to a stirred solution of LDA (2.5 eq.) in dry THF at −10° C. The solution was allowed to reach RT and then was stirred at 40° C. for 5 h. After cooled the reaction mixture at −10° C., it was quenched with dry MeOH (2.5 eq.) and stirring was continued at RT overnight. After evaporation of the solvent the resulting residue was taken up in DCM and washed with 0.1N HCl and brine. The combined organic fractions were dried (Na₂SO₄) and solvent was evaporated under reduced pressure giving a the crude compound as a 1:1 mixture of isomer that was used in the next step without further purification.

### Step 4: tert-butyl 3-methyl-4-(3-quinolin-2-yl-1,2,4-oxadiazol-5-yl)piperidine-1-carboxylate (D4)

A solution of (D3) and CDI (1.2 eq.) in DMF (0.3 M) was stirred at RT for 30 min. Then, N′-hydroxyquinoline-2-carboximidamide (1 eq.) was added and the resulting solution was stirred at RT overnight. Afterwards, more CDI (1.1 eq.) was added and the resulting solution was heated to 115° C. for 12 h. After cooling down, the reaction mixture was extracted with DCM, washed with 1N HCl 1N, sat. sol. NaHCO₃ and brine. The combined organic fractions were dried (Na₂SO₄) and the solvent was evaporated under reduced pressure giving a residue which was purified by flash column chromatography on silica using a gradient of EtOAc/Petroleum ether from 20:80 to 30:70 EtOAc to yield (17%) the title compound as a yellow oil. ¹H NMR (400 MHz, CDCl₃, 300 K) δ 8.39-8.29 (2H, m), 8.25-8.20 (1H, m), 7.93-7.86 (1H, m), 7.84-7.75 (1H, m), 7.69-7.58 (1H, m), 4.3-4.18 (1H, m), 3.88-3.75 (1H, m), 3.54-3.43 (1H, m), 3.40-3.28 (1H, m), 3.03-2.81 (1H, m), 2.51-2.38 (1H, m), 2.29-2.13 (1H, m), 2.04-1.97 (1H, m), 1.49 (9H, s), 0.94 (3H, d, J=6.8 Hz). MS (ES⁺) C₂₂H₂₆N₄O₃ requires: 394, found: 395 (M+H)⁺.

### Step 5: 3-methyl-4-(3-quinolin-2-yl-1,2,4-oxadiazol-5-yl)piperidinium trifluoroacetate (D5)

A solution of (D4) in TFA/DCM (1:9, 0.1 M) was stirred at RT for 3 h. Solvents were evaporated under reduced pressure to yield quantitatively the title compound that was used in the next step without further purification. MS (ES⁺) C₁₇H₁₈N₄O₂ requires: 294, found: 295 (M+H)⁺.

### Step 6: N-(2-chlorophenyl)-3-methyl-4-(3-quinolin-2-yl-1,2,4-oxadiazol-5-yl)piperidine-1-carboxamide (D6)

(D6) was prepared following the general procedure reported in Example 1 step 2. The corresponding crude was purified by flash column chromatography on silica using a gradient of EtOAc/Petroleum ether from 20:80 to 50:50 to yield (46%) the title compound as a white powder. ¹H NMR (400 MHz, DMSO-d₆, 300 K) δ 8.66-8.58 (1H, m), 8.25-8.16 (3H, m), 8.14-8.07 (1H, m), 7.94-7.84 (1H, m), 7.79-7.70 (1H, m), 7.55-7.41 (2H, m), 7.35-7.26 (1H, m), 7.21-7.10 (1H, m), 4.27-4.03 (2H, m), 3.91-3.79 (1H, m), 3.74-3.61 (1H, m), 3.51-3.39 (1H, m), 2.45-2.36 (1H, m), 2.19-1.96 (2H, m), 0.88 (3H, d, J=6.9 Hz). MS (ES⁺) C₂₄H₂₂ClN₅O₂ requires: 447, found: 448 (M+H)⁺.

### EXAMPLE 5

### Cyclohexyl 4-(3-quinolin-2-yl-1,2,4-oxadiazol-5-yl)piperidine-1-carboxylate (E1)

To a solution of triphosgene (0.5 eq.) in dry DCE (0.06 M) at 0° C., a solution of cyclohexanol (1.5 eq.) and TEA (1.5 eq.) in DCE (0.8 M) was added. The reaction mixture was stirred at 0° C. for 1 h. The reaction solution was then added dropwise to a stirring solution of (A1) and TEA (2.2 eq.) in DCE/DMF (5:1, 1.5 M) at 0° C. The resulting solution was stirred overnight at RT. The reaction mixture was diluted with EtOAc and washed with brine. Evaporation of the solvent under reduced pressure gave a residue which was purified by flash column chromatography on silica using a gradient of EtOAc/Petroleum ether from 90:10 to 100% EtOAc to yield (48%) the title compound as an oil. ¹H NMR (400 MHz, DMSO-d₆, 300 K) δ 8.64 (1H, d, J=8.8 Hz),), 8.23-8.20 (2H, m), 8.14 (1H, d, J=7.9 Hz), 7.92 (1H, t, J=8.2 Hz), 7.77 (1H, t, J=7.9 Hz), 4.65-4.60 (1H, m), 4.08-4.04 (2H, m), 3.52-3.46 (1H, m), 3.20-3.05 (2H, m), 2.21-2.17 (2H, m), 1.82-1.68 (6H, m), 1.51-1.27 (6H, m). MS (ES⁺) C₂₃H₂₆N₄O₃ required: 406, found: 407 (M+H)⁺.

### EXAMPLE 6

### 2-{5-[1-(Cycloheptylacetyl)piperidin-4-yl]-1,2,4-oxadiazol-3-yl}quinoline (F1)

A solution of cycloheptylacetic acid, TBTU (1.2 eq.) and DIPEA (1.2 eq.) in DMF (0.47 M) was stirred at RT for 5 min. Afterwards, a solution of (A1) (1.1 eq.) and DIPEA (3.2 eq.) in DMF (0.47 M) was added. The reaction mixture was stirred at RT overnight. Then, the reaction mixture was diluted with DCM and washed with brine and dried (Na₂SO₄). Evaporation of the solvent under reduced pressure gave a residue which was purified by flash column chromatography on silica using a gradient of EtOAc/Petroleum ether from 40:60 to 100% EtOAc to yield (79%) the title compound as a salmon powder. ¹H NMR (400 MHz, DMSO-d₆, 300 K) δ 8.64 (1H, d, J=7.9 Hz), 8.21 (2H, d, J=7.7 Hz), 8.13 (1H, d, J=7.0 Hz), 7.98-7.90 (1H, m), 7.85-7.74 (1H, m), 4.42-4.39 (1H, m), 4.01-3.98 (1H, m), 3.51-3.48 (1H, m), 3.30-3.27 (1H, m), 2.92-2.88 (1H, m), 2.30-2.18 (4H, m), 1.96-1.46 (13H, m), 1.24-1.20 (2H, m). MS (ES⁺) C₂₅H₃₀N₄O₂ required: 418, found: 419 (M+H)⁺.

### EXAMPLE 7

### N-(3,5-Dichlorophenyl)-4-(3-quinolin-2-yl-1,2,4-oxadiazol-5-yl)piperidine-1-sulfonamide (G3)

### Step 1: 2-{5-[1-(1H-Imidazol-1-ylsulfonyl)piperidin-4-yl]-1,2,4-oxadiazol-3-yl}quinoline (G1)

To a solution of 1-(1H-imidazol-1-ylsulfonyl)-3-methyl-1H-imidazol-3-ium trifluoroacetate (1.5 eq.), prepared as described in Beaudoin, S. et al. J. Org. Chem. 2003, 68, 115, in MeCN (0.17 M) was added 2-(5-piperidin-4-yl-1,2,4-oxadiazol-3-yl)quinoline and the reaction mixture was stirred at RT for 2 days. Then, the reaction mixture was diluted with EtOAc and washed with brine and dried (Na₂SO₄). Evaporation of the solvent under reduced pressure gave a residue which was purified by flash column chromatography on silica using a gradient of EtOAc/Petroleum ether from 25:75 to 100% EtOAc to yield (71%) the title compound as a white powder. ¹H NMR (400 MHz, CDCl₃, 300 K) δ 8.32 (2H, d, J=8.4 Hz), 8.18 (1H, d, J=8.5 Hz), 7.93 (1H, bs), 7.88 (1H, dd, J=8.0 and 1.2 Hz), 7.79 (1H, ddd, J=8.4, 7.0 and 1.4 Hz), 7.64 (1H, ddd, J=8.0, 7.0 and 1.0 Hz), 7.27 (1H, t, J=1.4 Hz), 7.18-7.17 (1H, m), 3.90 (2H, ddd, J=12.5 and 3.8 Hz), 3.22-3.15 (1H, m), 2.92-2.86 (2H, m), 2.37-2.32 (2H, m), 2.24-2.14 (2H, m). MS (ES⁺) C₁₉H₁₈N₆O₃S required: 410, found: 411 (M+H)⁺.

### Step 2: 3-Methyl-1-{[4-(3-quinolin-2-yl-1,2,4-oxadiazol-5-yl)piperidin-1-yl]sulfonyl}-1H-imidazol-3-ium trifluoroacetate (G2)

To a solution of (G1) in DCM (0.3 M) at 0° C., methyl triflate (1.16 eq.) was added. The reaction mixture was stirred at 0° C. for 2 h and then, it was concentrated under reduced pressure to give the title compound which was used as such in the next step. MS (ES⁺) C₂₀H₂₁N₆O₃S⁺ required: 425, found: 425 (M⁺).

### Step 3: 3N-(3,5-Dichlorophenyl)-4-(3-quinolin-2-yl-1,2,4-oxadiazol-5-yl)piperidine-1-sulfonamide (G3)

A solution of (G2), 3,5-dichloroaniline (1.2 eq.) and TEA (1 eq.) was heated to 80° C. overnight. After cooling down, the reaction mixture was concentrated under reduced pressure to give a residue which was purified by flash column chromatography on silica using a gradient of EtOAc/Petroleum ether from 50:50 to 100% EtOAc to yield the title compound as a yellow powder. ¹H NMR (400 MHz, CDCl₃, 300 K) δ 10.57 (1H, bs), 8.64 (1H, d, J=8.5 Hz), 8.22-8.19 (2H, m), 8.14 (1H, d, J=8.4 Hz), 7.93-7.90 (1H, m), 7.79-7.75 (1H, m), 7.27 (1H, s), 7.19 (2H, s), 3.74-3.69 (2H, m), 3.4 (1H, masked), 3.09-3.03 (2H, m), 2.25-2.21 (2H, m), 1.85-1.76 (2H, m). MS (ES⁺) C₂₂H₁₉Cl₂N₅O₃S required: 503, found: 504/506 (M+H)⁺.

### EXAMPLE 8

### 2-[5-(1-{[(1,1-Dioxidotetrahydro-2H-thiopyran-4-yl)amino]carbonyl}piperidin-4-yl)-1,2,4-oxadiazol-3-yl]quinolinium trifluoroacetate (H1)

To a solution of (A1) and DIPEA (4 eq.) in DCM (0.02 M) at −10° C., triphosgene (1.1 eq.) was added. The reaction mixture was stirred at −10° C. for 20 min. Then, a solution of tetrahydro-2H-thiopyran-4-amine 1,1-dioxide (1.15 eq.) and DIPEA (2 eq.) in DMSO (0.09 M) was added and the resulting solution was heated to reflux overnight. After cooling down, the reaction mixture was concentrated to give a crude which was purified by preparative RP-HPLC, using water (+0.1% TFA) and MeCN (+0.1% TFA) as eluents (column: C18). The desired fractions were lyophilized to yield (41%) the title compound as a white powder. ¹H NMR (400 MHz, MeOD-d₄, 300 K) δ 8.56 (1H, d, J=8.4 Hz), 8.24-8.21 (2H, m), 8.01 (1H, d, J=8.4 Hz), 7.86 (1H, d, J=7.6 Hz), 7.69 (1H, t, J=7.6 Hz), 4.08 (2H, d, J=13.6 Hz), 3.90-3.86 (1H, m), 3.41-3.36 (1H, m), 3.29-3.20 (2H, m), 3.09-3.02 (4H, m), 2.22-2.08 (6H, m), 1.92-1.83 (2H, m). MS (ES⁺) C₂₂H₂₅N₅O₄S required: 455, found: 456 (M+H)⁺.

### EXAMPLE 9

### 2-(5-{1-[(cyclohexylamino)carbonyl]piperidin-4-yl}-1,2,4-oxadiazol-3-yl)-1-methyl-1H-3,1-benzimidazol-1-ium trifluoroacetate (14)

### Step 1: tert-butyl 4-[3-(1H-benzimidazol-2-yl)-1,2,4-oxadiazol-5-yl]piperidine-1-carboxylate (I1)

(I1) was prepared following the general procedure reported in Example 1 step 1 starting from 1H-benzimidazole-2-carbonitrile and 1-(tert-butoxycarbonyl)piperidine-4-carboxylic acid to yield the title compound as a yellow oil. ¹H NMR (300 MHz, DMSO-d6, 300 K) δ 13.48 (1H, s), 7.76 (1H, d, J=8.05 Hz), 7.58 (1H, d, J=8.05 Hz), 7.39-7.23 (2H, m), 4.03-3.87 (2H, m), 3.49-3.36 (1H, m), 3.09-2.96 (2H, m), 2.19-2.06 (2H, m), 1.83-1.64 (2H, m), 1.42 (9H, s). MS (ES⁺) C₁₉H₂₃N₅O₃ required: 369, found: 370 (M+H)⁺.

### Step 2: tert-butyl 4-[3-(1-methyl-1H-benzimidazol-2-yl)-1,2,4-oxadiazol-5-yl]piperidine-1-carboxylate (I2)

To a stirred solution of (I1) in acetone (0.2 M) was added at RT powdered potassium hydroxide (10 eq.) and, after a few minutes, methyl iodide (6 eq.). The reaction mixture was stirred at RT for 40 min, then solvent was evaporated to dryness and the resulting residue was partitioned between DCM and brine. The organic phase was dried (Na₂SO₄), filtered and solvent was evaporated under reduced pressure to afford the title compound as a yellow solid in 85% yield. ¹H NMR (400 MHz, DMSO-d6, 300 K) δ 7.78 (1H, d, J=8.07 Hz), 7.73 (1H, d, J=8.07 Hz), 7.46-7.39 (1H, m), 7.37-7.30 (1H, m), 4.13 (3H, s), 4.00-3.89 (2H, m), 3.49-3.39 (1H, m), 3.06-2.95 (2H, m), 2.16-2.06 (2H, m), 1.82-1.64 (2H, m), 1.42 (9H, s). MS (ES⁺) C₂₀H₂₅N₅O₃ required: 383, found: 384 (M+H)⁺.

### Step 3: 1-methyl-2-(5-piperidinium-4-yl-1,2,4-oxadiazol-3-yl)-1H-3,1-benzimidazol-1-ium bis(trifluoroacetate) (I3)

(I3) was prepared from (I2) following the general procedure reported in Example 4 step 5. MS (ES⁺) C₁₅H₁₇N₅O required: 283, found: 284 (M+H)⁺.

### Step 4: 2-(5-{1-[(cyclohexylamino)carbonyl]piperidin-4-yl}-1,2,4-oxadiazol-3-yl)-1-methyl-1H-3,1-benzimidazol-1-ium trifluoroacetate (I4)

(I4) was prepared following the general procedure reported in Example 2 step 1 using isocyanatocyclohexane (1 eq.). The corresponding crude was purified by preparative RP-HPLC using water (+0.1% TFA) and MeCN (+0.1% TFA) as eluents (column: C18). The desired fractions were lyophilized to yield (44%) the title compound as a yellow oil. ¹H NMR (300 MHz, DMSO-d6, 300 K) δ 7.78 (1H, d, J=8.05 Hz), 7.74 (1H, d, J=8.05 Hz), 7.47-7.39 (1H, m), 7.38-7.31 (1H, m), 6.20 (1H, bs), 4.14 (3H, s), 4.00-3.91 (2H, m), 3.48-3.33 (1H, m), 3.00-2.86 (2H, m), 2.14-2.03 (2H, m), 1.81-1.62 (6H, m), 1.61-1.50 (1H, m), 1.32-1.02 (5H, m). MS (ES⁺) C₂₂H₂₈N₆O₂ required: 408, found: 409 (M+H)⁺.

### EXAMPLE 10

### 1-methyl-2-{5-[1-({[(1-morpholin-4-ium-4-ylcyclopentyl)methyl]amino}carbonyl)piperidin-4-yl]-1,2,4-oxadiazol-3-yl}-1H-3,1-benzimidazol-1-ium bis(trifluoroacetate) (J1)

A solution of 1-(1-morpholin-4-ylcyclopentyl)methanamine (5 eq.), CDI (5.5 eq.) and TEA (25 eq.) in dry THF (0.1 M) was stirred for two hours at 50° C. The solution was cooled to RT and then a solution of (I3) and TEA (5 eq.) in THF (0.1M) was added. The resulting mixture was stirred at RT overnight. After evaporation of the solvent the product was directly purified by preparative RP-HPLC using water (+0.1% TFA) and MeCN (+0.1% TFA) as eluents (column: C18). The desired fractions were lyophilized to yield (10%) the title compound as a colourless solid. ¹H NMR (300 MHz, DMSO-d6, 300 K) δ 9.64 (1H, m), 7.83-7.68 (2H, m), 7.48-7.39 (1H, m), 7.38-7.29 (1H, m), 7.04-6.90 (1H, m), 4.13 (3H, s), 4.08-3.95 (4H partially under water signal, m), 3.76-3.61 (2H partially under water signal, m), 3.56-3.25 (7H, m), 3.16-2.98 (2H, m), 2.21-2.06 (2H, m), 1.95-1.81 (4H, m), 1.80-1.60 (6H, m). MS (ES⁺) C₂₆H₃₅N₇O₃ required: 493, found: 494 (M+H)⁺.

### EXAMPLE 11

### 2-(5-{4-[(Cyclohexylamino)carbonyl]piperazin-1-yl}-1,2,4-oxadiazol-3-yl)quinolinium trifluoroacetate (K4)

### Step 1: 2-[5-(Trichloromethyl)-1,2,4-oxadiazol-3-yl]quinoline (K1)

Trichloroacetyl chloride (3 eq.) was added dropwise to a mixture of commercially available N′-hydroxyquinoline-2-carboximidamide and trichloroacetic acid (3.6 eq.) heated to 90° C. Then, the reaction mixture was heated to 90° C. overnight. After cooling down, the reaction mixture was partitioned between water and EtOAc. The organic phase was washed with brine and dried (Na₂SO₄). Evaporation of the solvent under reduced pressure gave a residue that was triturated in water/MeCN. The resulting precipitate was filtered and dried to yield (80%) the title compound as a beige powder. ¹H NMR (300 MHz, CDCl₃, 300 K) δ 8.40-8.33 (2H, m), 8.25 (1H, d, J=8.6 Hz), 7.91 (1H, d, J=8.3 Hz), 7.85-7.80 (1H, m), 7.70-7.64 (1H, m). MS (ES⁺) C₁₂H₆N₃Cl₃O required: 313, found: 314/316 (M+H)⁺.

### Step 2: Tert-butyl 4-(3-quinolin-2-yl-1,2,4-oxadiazol-5-yl)piperazine-1-carboxylate (K2)

To a solution of (K1) in dry THF (0.5 M) at RT, 1-(3,3-dimethylbutanoyl)piperazine was added and the reaction mixture was stirred at RT overnight. Then, the solution was diluted in EtOAc and washed with brine and dried (Na₂SO₄). Evaporation of the solvent under reduced pressure gave a residue which was purified by flash column chromatography on silica using a gradient of EtOAc/Petroleum ether from 30:70 to 100% EtOAc to afford the title compound. ¹H NMR (400 MHz, CDCl₃, 300 K) δ 8.31 (1H, d, J=8.5 Hz), 8.27 (1H, d, J=8.5 Hz), 8.13 (1H, d, J=8.5 Hz), 7.86 (1H, d, J=8.0 Hz), 7.76 (1H, ddd, J=8.2, 6.9 and 1.2 Hz), 7.62-7.58 (1H, m), 3.78-3.75 (4H, m), 3.61-3.58 (4H, m), 1.50 (9H, s). MS (ES⁺) C₂₀H₂₃N₅O₃ required: 381, found: 382 (M+H)⁺.

### Step 3: 2-(5-piperazin-4-ium-1-yl-1,2,4-oxadiazol-3-yl)quinolinium bis(trifluoroacetate) (K3)

A solution of (K2) in DCM/TFA (2:1, 0.2 M) was stirred for 3 h at RT. Then, the solution was concentrated under reduced pressure and the residue was dissolved in a mixture of MeCN/H₂O (1:1) and lyophilized to afford the title compound as a yellow oil. MS (ES⁺) C₁₅H₁₅N₅O required: 281, found: 282 (M+H)⁺.

### Step 4: 2-(5-{4-[(Cyclohexylamino)carbonyl]piperazin-1-yl}-1,2,4-oxadiazol-3-yl)quinolinium trifluoroacetate (K4)

To a solution of (K3) and DIPEA (4 eq.) in dry DCE/DMF (5:1, 0.3 M) at 0° C., isocyanatocyclohexane (2 eq.) was added. The reaction suspension was stirred at RT overnight. Then, the reaction mixture was concentrated and the crude dissolved with EtOAc. The resulting white precipitate was filtered off and the filtrate was concentrated. The resulting residue crude was re-dissolved in DMF and purified by prep. HPLC [eluting phase: MeCN (0.1% of TFA) and H₂O (0.1% TFA)]. The product fractions were lyophilized to afford the title compound as a yellow powder. ¹H NMR (300 MHz, CDCl₃, 300 K) δ 8.55 (1H, d, J=8.4 Hz), 8.15 (1H, d, J=8.4 Hz), 8.10 (1H, d, J=8.4 Hz), 8.08 (1H, d, J=7.7 Hz), 7.88-7.84 (1H, m), 7.73-7.69 (1H, m), 6.36 (1H, bs), 3.64-3.62 (4H, m), 3.50-3.47 (4H, m), 1.79-1.68 (4H, m), 1.59-1.56 (1H, m), 1.30-1.04 (6H, m). MS (ES⁺) C₂₂H₂₆N₆O₂ required: 406, found: 407 (M+H)⁺.

The compounds in the following tables were made according to the procedures described above.

TABLE 1
Ureidopiperidineoxadiazoles

| | | Molecular Ion | Procedure of |
|---|---|---|---|
| Example | Name | [M + H]⁺ | Example |
| 12 | 4-{1-[({[4-(3-quinolin-2-yl-1,2,4-oxadiazol- | 491 | 8 |
| | 5-yl)piperidin-1- | | |
| | yl]carbonyl}amino)methyl]cyclopentyl}morpholin- | | |
| | 4-ium trifluoroacetate | | |
| 13 | N-[(1R,2S)-2-phenylcyclopropyl]-4-(3- | 440 | 1 |
| | quinolin-2-yl-1,2,4-oxadiazol-5-yl)piperidine- | | |
| | 1-carboxamide | | |
| 14 | N-(3,4-dichlorophenyl)-4-(3-quinolin-2-yl- | 468/470/472 | 1 |
| | 1,2,4-oxadiazol-5-yl)piperidine-1- | | |
| | carboxamide | | |
| 15 | N-biphenyl-2-yl-4-(3-quinolin-2-yl-1,2,4- | 476 | 1 |
| | oxadiazol-5-yl)piperidine-1-carboxamide | | |
| 16 | N-benzyl-4-(3-quinolin-2-yl-1,2,4-oxadiazol- | 414 | 1 |
| | 5-yl)piperidine-1-carboxamide | | |
| 17 | N-[3,5-bis(trifluoromethyl)phenyl]-4-(3- | 536 | 1 |
| | quinolin-2-yl-1,2,4-oxadiazol-5-yl)piperidine- | | |
| | 1-carboxamide | | |
| 18 | N-(2-methoxyphenyl)-4-(3-quinolin-2-yl- | 430 | 1 |
| | 1,2,4-oxadiazol-5-yl)piperidine-1- | | |
| | carboxamide | | |
| 19 | N-[3,5-bis(trifluoromethyl)phenyl]-4-(3- | 486 | 1 |
| | pyridin-2-yl-1,2,4-oxadiazol-5-yl)piperidine- | | |
| | 1-carboxamide | | |
| 20 | N-phenyl-4-(3-quinolin-2-yl-1,2,4-oxadiazol- | 400 | 1 |
| | 5-yl)piperidine-1-carboxamide | | |
| 21 | N-[(1S)-1-phenylethyl]-4-(3-quinolin-2-yl- | 428 | 1 |
| | 1,2,4-oxadiazol-5-yl)piperidine-1- | | |
| | carboxamide | | |
| 22 | N-(2,5-dichlorophenyl)-4-(3-quinolin-2-yl- | 469 | 1 |
| | 1,2,4-oxadiazol-5-yl)piperidine-1- | | |
| | carboxamide | | |
| 23 | N-(4-chlorophenyl)-4-(3-quinolin-2-yl-1,2,4- | 434 | 1 |
| | oxadiazol-5-yl)piperidine-1-carboxamide | | |
| 24 | N-(2-isopropylphenyl)-4-(3-quinolin-2-yl- | 442 | 1 |
| | 1,2,4-oxadiazol-5-yl)piperidine-1- | | |
| | carboxamide | | |
| 25 | N-(2,5-dichlorophenyl)-4-[3-(3- | 432/434/436 | 3 |
| | methylpyridin-2-yl)-1,2,4-oxadiazol-5- | | |
| | yl]piperidine-1-carboxamide | | |
| 26 | N-(2-isopropylphenyl)-4-[3-(3-methylpyridin- | 406 | 3 |
| | 2-yl)-1,2,4-oxadiazol-5-yl]piperidine-1- | | |
| | carboxamide | | |
| 27 | N-(2,5-dichlorophenyl)-4-(3-pyridin-2-yl- | 418/420/421 | 3 |
| | 1,2,4-oxadiazol-5-yl)piperidine-1- | | |
| | carboxamide | | |
| 28 | N-(4-methoxyphenyl)-4-[3-(3-methylpyridin- | 394 | 3 |
| | 2-yl)-1,2,4-oxadiazol-5-yl]piperidine-1- | | |
| | carboxamide | | |
| 29 | N-biphenyl-2-yl-4-[3-(3-methylpyridin-2-yl)- | 440 | 3 |
| | 1,2,4-oxadiazol-5-yl]piperidine-1- | | |
| | carboxamide | | |
| 30 | N-[3,5-bis(trifluoromethyl)phenyl]-4-[3-(3- | 500 | 3 |
| | methylpyridin-2-yl)-1,2,4-oxadiazol-5- | | |
| | yl]piperidine-1-carboxamide | | |
| 31 | N-(2-methoxyphenyl)-4-[3-(3-methylpyridin- | 394 | 3 |
| | 2-yl)-1,2,4-oxadiazol-5-yl]piperidine-1- | | |
| | carboxamide | | |
| 32 | N-(3-methoxyphenyl)-4-(3-quinolin-2-yl- | 430 | 1 |
| | 1,2,4-oxadiazol-5-yl)piperidine-1- | | |
| | carboxamide | | |
| 33 | N-(4-methoxyphenyl)-4-(3-quinolin-2-yl- | 430 | 1 |
| | 1,2,4-oxadiazol-5-yl)piperidine-1- | | |
| | carboxamide | | |
| 34 | N-(2-chlorophenyl)-4-[3-(3-methylpyridin-2- | 398 | 3 |
| | yl)-1,2,4-oxadiazol-5-yl]piperidine-1- | | |
| | carboxamide | | |
| 35 | N-(3,5-dichlorophenyl)-4-[3-(3- | 432/434/436 | 3 |
| | methylpyridin-2-yl)-1,2,4-oxadiazol-5- | | |
| | yl]piperidine-1-carboxamide | | |
| 36 | 4-[3-(3-methylpyridin-2-yl)-1,2,4-oxadiazol- | 456 | 3 |
| | 5-yl]-N-(4-phenoxyphenyl)piperidine-1- | | |
| | carboxamide | | |
| 37 | N-(4-chlorophenyl)-4-[3-(3-methylpyridin-2- | 398/400 | 3 |
| | yl)-1,2,4-oxadiazol-5-yl]piperidine-1- | | |
| | carboxamide | | |
| 38 | 3-methyl-2-{5-[1-({[4- | 432 | 3 |
| | (trifluoromethyl)phenyl]amino}carbonyl)piperidin- | | |
| | 4-yl]-1,2,4-oxadiazol-3-yl}pyridinium | | |
| | trifluoroacetate | | |
| 39 | N-(2-methylphenyl)-4-(3-quinolin-2-yl-1,2,4- | 414 | 1 |
| | oxadiazol-5-yl)piperidine-1-carboxamide | | |
| 40 | N,N-dimethyl-4-(3-quinolin-2-yl-1,2,4- | 352 | 1 |
| | oxadiazol-5-yl)piperidine-1-carboxamide | | |
| 41 | N-2-naphthyl-4-(3-quinolin-2-yl-1,2,4- | 450 | 1 |
| | oxadiazol-5-yl)piperidine-1-carboxamide | | |
| 42 | N-cyclohexyl-4-(3-quinolin-2-yl-1,2,4- | 406 | 1 |
| | oxadiazol-5-yl)piperidine-1-carboxamide | | |
| 43 | N-(2,6-dichlorophenyl)-4-(3-quinolin-2-yl- | 468 | 1 |
| | 1,2,4-oxadiazol-5-yl)piperidine-1- | | |
| | carboxamide | | |
| 44 | N-1H-indol-3-yl-4-(3-quinolin-2-yl-1,2,4- | 439 | 1 |
| | oxadiazol-5-yl)piperidine-1-carboxamide | | |
| 45 | 3-[5-(1-{[(2- | 430 | 1 |
| | methoxyphenyl)amino]carbonyl}piperidin-4- | | |
| | yl)-1,2,4-oxadiazol-3-yl]isoquinolinium | | |
| | trifluoroacetate | | |
| 46 | 2-[5-(1-{[(2- | 444 | 1 |
| | methoxyphenyl)(methyl)amino]carbonyl}piperidin- | | |
| | 4-yl)-1,2,4-oxadiazol-3-yl]quinolinium | | |
| | trifluoroacetate | | |
| 47 | 2-{5-[1-(3,4-dihydroquinolin-1(2H)- | 440 | 1 |
| | ylcarbonyl)piperidin-4-yl]-1,2,4-oxadiazol-3- | | |
| | yl}quinolinium trifluoroacetate | | |
| 48 | N-(3-methyl-2-furyl)-4-(3-quinolin-2-yl- | 404 | 1 |
| | 1,2,4-oxadiazol-5-yl)piperidine-1- | | |
| | carboxamide | | |
| 49 | N-(3,5-dichlorophenyl)-4-(3-quinolin-2-yl- | 470/468 | 1 |
| | 1,2,4-oxadiazol-5-yl)piperidine-1- | | |
| | carboxamide | | |
| 50 | 2-[5-(1-{[(2,3- | 468/470/472 | 1 |
| | dichlorophenyl)amino]carbonyl}piperidin-4- | | |
| | yl)-1,2,4-oxadiazol-3-yl]quinolinium | | |
| | trifluoroacetate | | |
| 51 | 2-[5-(1-{[(6-chloropyridin-3- | 438/440 | 1 |
| | yl)amino]carbonyl}piperidin-4-yl)-1,2,4- | | |
| | oxadiazol-3-yl]quinolinium trifluoroacetate | | |
| 52 | 4-[3-({[4-(3-quinolin-2-yl-1,2,4-oxadiazol-5- | 502 | 1 |
| | yl)piperidin-1- | | |
| | yl]carbonyl}amino)benzyl]morpholin-4-ium | | |
| | trifluoroacetate | | |
| 53 | 2-(5-{1-[(2,3-dihydro-1,4-benzodioxin-6- | 458 | 1 |
| | ylamino)carbonyl]piperidin-4-yl}-1,2,4- | | |
| | oxadiazol-3-yl)quinolinium trifluoroacetate | | |
| 54 | 2-[5-(1-{[(3- | 425 | 1 |
| | cyanophenyl)amino]carbonyl}piperidin-4-yl)- | | |
| | 1,2,4-oxadiazol-3-yl]quinolinium | | |
| | trifluoroacetate | | |
| 55 | 2-(5-{1-[(1-benzofuran-6- | 440 | 1 |
| | ylamino)carbonyl]piperidin-4-yl}-1,2,4- | | |
| | oxadiazol-3-yl)quinolinium trifluoroacetate | | |
| 56 | 2-[5-(1-{[(4-chloro-2- | 452 | 1 |
| | fluorophenyl)amino]carbonyl}piperidin-4-yl)- | | |
| | 1,2,4-oxadiazol-3-yl]quinolinium | | |
| | trifluoroacetate | | |
| 57 | 2-[5-(1-{[(5-fluoro-2- | 432 | 1 |
| | methylphenyl)amino]carbonyl}piperidin-4- | | |
| | yl)-1,2,4-oxadiazol-3-yl]quinolinium | | |
| | trifluoroacetate | | |
| 58 | 2-[5-(1-{[(2-chlorophenyl)amino]carbonyl}- | 448 | 4 |
| | 4-methylpiperidin-4-yl)-1,2,4-oxadiazol-3- | | |
| | yl]quinolinium trifluoroacetate | | |
| 59 | 2-[5-(1-{[(2-chlorophenyl)amino]carbonyl}- | 448 | 4 |
| | 2-methylpiperidin-4-yl)-1,2,4-oxadiazol-3- | | |
| | yl]quinolinium trifluoroacetate | | |
| 60 | 2-[5-(1-{[(2-chlorophenyl)amino]carbonyl}- | 450 | 4 |
| | 4-hydroxypiperidin-4-yl)-1,2,4-oxadiazol-3- | | |
| | yl]quinolinium trifluoroacetate | | |
| 61 | 2-{5-[1-({[2- | 468 | 4 |
| | (trifluoromethyl)phenyl]amino}carbonyl)piperidin- | | |
| | 4-yl]-1,2,4-oxadiazol-3-yl}quinolinium | | |
| | trifluoroacetate | | |
| 62 | 2-[5-(1-{[(2-chlorophenyl)amino]carbonyl}- | 452 | 4 |
| | 4-fluoropiperidin-4-yl)-1,2,4-oxadiazol-3- | | |
| | yl]quinolinium trifluoroacetate | | |
| 63 | 2-{5-[1-({[2-(pyridin-2- | 444 | 8 |
| | ylamino)ethyl]amino}carbonyl)piperidin-4- | | |
| | yl]-1,2,4-oxadiazol-3-yl}quinolinium | | |
| | trifluoroacetate | | |
| 64 | trans-N-(2-chlorophenyl)-2-methyl-4-(3- | 448 | 1 |
| | quinolin-2-yl-1,2,4-oxadiazol-5-yl)piperidine- | | |
| | 1-carboxamide | | |
| 65 | cis-N-(2-chlorophenyl)-2-methyl-4-(3- | 448 | 1 |
| | quinolin-2-yl-1,2,4-oxadiazol-5-yl)piperidine- | | |
| | 1-carboxamide | | |
| 66 | 2-[5-(1- | 392 | 8 |
| | {[(cyclobutylmethyl)amino]carbonyl}piperidin- | | |
| | 4-yl)-1,2,4-oxadiazol-3-yl]quinolinium | | |
| | trifluoroacetate | | |
| 67 | 2-[5-(1-{[(3-chloropyridin-2- | 435 | 1 |
| | yl)amino]carbonyl}piperidin-4-yl)-1,2,4- | | |
| | oxadiazol-3-yl]quinolinium trifluoroacetate | | |
| 68 | 2-(5-{1-[(tetrahydro-2H-pyran-4- | 408 | 8 |
| | ylamino)carbonyl]piperidin-4-yl}-1,2,4- | | |
| | oxadiazol-3-yl)quinolinium trifluoroacetate | | |
| 69 | N,N,3-trimethyl-3-({[4-(3-quinolin-2-yl-1,2,4- | 451 | 8 |
| | oxadiazol-5-yl)piperidin-1- | | |
| | yl]carbonyl}amino)pentan-1-aminium | | |
| | trifluoroacetate | | |
| 70 | 8-[(methyl{[4-(3-quinolin-2-yl-1,2,4- | 479 | 8 |
| | oxadiazol-5-yl)piperidin-1- | | |
| | yl]carbonyl}amino)methyl]quinolinium | | |
| | trifluoroacetate | | |
| 71 | 2-{5-[1-({[1-(methylsulfonyl)piperidin-4- | 485 | 8 |
| | yl]amino}carbonyl)piperidin-4-yl]-1,2,4- | | |
| | oxadiazol-3-yl}quinolinium trifluoroacetate | | |
| 72 | N-phenyl-4-(3-quinolin-3-yl-1,2,4-oxadiazol- | 400 | 2 |
| | 5-yl)piperidine-1-carboxamide | | |
| 73 | N-(2-chlorophenyl)-4-(3-quinolin-3-yl-1,2,4- | 434/436 | 2 |
| | oxadiazol-5-yl)piperidine-1-carboxamide | | |
| 74 | N-(3,5-dichlorophenyl)-4-(3-quinolin-3-yl- | 468/470 | 2 |
| | 1,2,4-oxadiazol-5-yl)piperidine-1- | | |
| | carboxamide | | |
| 75 | N-(4-chlorophenyl)-4-(3-quinolin-3-yl-1,2,4- | 434/436 | 2 |
| | oxadiazol-5-yl)piperidine-1-carboxamide | | |
| 76 | N-(3-methoxyphenyl)-4-(3-quinolin-3-yl- | 430 | 2 |
| | 1,2,4-oxadiazol-5-yl)piperidine-1- | | |
| | carboxamide | | |
| 77 | N-1-naphthyl-4-(3-quinolin-3-yl-1,2,4- | 450 | 2 |
| | oxadiazol-5-yl)piperidine-1-carboxamide | | |
| 78 | N-(2-methoxyphenyl)-4-(3-quinolin-3-yl- | 430 | 2 |
| | 1,2,4-oxadiazol-5-yl)piperidine-1- | | |
| | carboxamide | | |
| 79 | N-(2-isopropylphenyl)-4-(3-quinolin-3-yl- | 442 | 2 |
| | 1,2,4-oxadiazol-5-yl)piperidine-1- | | |
| | carboxamide | | |
| 80 | N-(4-methoxyphenyl)-4-(3-quinolin-3-yl- | 430 | 2 |
| | 1,2,4-oxadiazol-5-yl)piperidine-1- | | |
| | carboxamide | | |
| 81 | N-(3,4-dichlorophenyl)-4-(3-quinolin-3-yl- | 468/470/472 | 2 |
| | 1,2,4-oxadiazol-5-yl)piperidine-1- | | |
| | carboxamide | | |

TABLE 2
Carbamoylpiperidineoxadiazoles

| | | Molecular | Procedure |
|---|---|---|---|
| | | Ion | of |
| Example | Name | [M + H]⁺ | Example |
| 82 | 2-methoxyphenyl 4-(3-quinolin-2-yl- | 431 | 5 |
| | 1,2,4-oxadiazol-5-yl)piperidine- | | |
| | 1-carboxylate | | |

TABLE 3
Amidopiperidineoxadiazoles

| | | Molecular Ion | Procedure of |
|---|---|---|---|
| Example | Name | [M + H]⁺ | Example |
| 83 | 2-{5-[1-(phenylacetyl)piperidin-4-yl]-1,2,4- | 399 | 6 |
| | oxadiazol-3-yl}quinoline | | |
| 84 | 2-{5-[1-(2,3-dihydro-1H-inden-2- | 439 | 6 |
| | ylacetyl)piperidin-4-yl]-1,2,4-oxadiazol-3- | | |
| | yl}quinoline | | |
| 85 | 3-{[4-(3-quinolin-2-yl-1,2,4-oxadiazol-5- | 439 | 6 |
| | yl)piperidin-1-yl]carbonyl}indan-1-one | | |
| 86 | 2-{5-[1-(1-adamantylacetyl)piperidin-4-yl]- | 457 | 6 |
| | 1,2,4-oxadiazol-3-yl}quinoline | | |
| 87 | 2-{5-[1-(1,3-benzodioxol-5- | 443 | 6 |
| | ylacetyl)piperidin-4-yl]-1,2,4-oxadiazol-3- | | |
| | yl}quinoline | | |

TABLE 4
Benzimidazoles and benzothiazoles

| | | Molecular Ion | Procedure of |
|---|---|---|---|
| Example | Name | [M + H]⁺ | Example |
| 88 | 2-(5-{1- | 395 | 9 |
| | [(cyclohexylamino)carbonyl]piperidin-4-yl}- | | |
| | 1,2,4-oxadiazol-3-yl)-1H-benzimidazol-3-ium | | |
| | trifluoroacetate | | |
| 89 | 4-[3-(1,3-benzothiazol-2-yl)-1,2,4-oxadiazol- | 412 | 9 |
| | 5-yl]-N-cyclohexylpiperidine-1-carboxamide | | |
| 90 | 2-[5-(1-{[(1,1-dioxidotetrahydro-2H- | 459 | 9 |
| | thiopyran-4-yl)amino]carbonyl}piperidin-4- | | |
| | yl)-1,2,4-oxadiazol-3-yl]-1-methyl-1H- | | |
| | benzimidazol-3-ium trifluoroacetate | | |

### EXAMPLE 91

### 2-(5-{4-[(4,4-Difluorocyclohexyl)carbamoyl]piperazin-1-yl}-1,2,4-oxadiazol-3-yl)quinolinium trifluoroacetate (L1)

Triphosgene (0.33 eq.) was added to a stirred solution of (K3) and DIPEA (5 eq.) in DCM (0.15 M) at −20° C. The mixture was stirred at the same temperature for 20 min. Then, a solution of 4,4-difluorocyclohexanaminium chloride (1 eq.) and DIPEA (1 eq.) in DCM (0.024 M) was added and the resulting mixture was stirred at RT overnight. Evaporation of the solvent under reduced pressure gave a crude that was purified by preparative RP-HPLC, using water (+0.1% TFA) and MeCN (+0.1% TFA) as eluents (column: C₁₈). The desired fractions were lyophilized to yield (72%) the title compound as a white powder. ¹H NMR (400 MHz, DMSO-d₆, 300 K) δ 8.55 (1H, d, J=8.4 Hz), 8.15 (1H, d, J=8.4 Hz), 8.11-8.07 (2H, m), 7.88-7.84 (1H, m), 7.74-7.69 (1H, m), 6.47 (1H, d, J=7.2 Hz), 3.66-3.63 (5H, m), 3.52-3.49 (4H, m), 2.08-1.94 (7H, m), 1.89-1.76 (2H, m). MS (ES⁺) C₂₂H₂₄F₂N₆O₂ required: 442, found: 443 (M+H)⁺.

### EXAMPLE 92

### 4-[3-(5-Fluoroquinolin-2-yl)-1,2,4-oxadiazol-5-yl]-N-{[1-(piperidin-1-yl)cyclopentyl]methyl}piperidine-1-carboxamide (M6)

### Step 1: tert-Butyl 4-[3-(ethoxycarbonyl)-1,2,4-oxadiazol-5-yl]piperidine-1-carboxylate (M1)

Ethyl (2Z)-amino(hydroxyimino)ethanoate (1.1 eq.) was added to a solution of TBTU (1.2 eq.), 1-(tert-butoxycarbonyl)piperidine-4-carboxylic acid (1 eq.) and DIPEA (2 eq.) in DCM (0.2 M) at RT. The reaction mixture was stirred at RT overnight. After dilution with EtOAc, the organic phase was washed with sat. sol. NaHCO₃ and dried (Na₂SO₄). Evaporation of the solvent under reduced pressure afforded a white solid which was diluted with THF (0.2 M). The resulting solution was treated with TBAF (0.5 eq.) and the reaction mixture was heated to reflux overnight. Then, the reaction mixture was diluted with EtOAc and washed with sat. sol. NaHCO₃ and dried (Na₂SO₄). Evaporation of the solvent under reduced pressure gave a crude which was purified by flash chromatography on silica using EtOAc/Petroleum ether (1:1) as solvent to afford (49%) the title compound as a brown oil. MS (ES⁺) C₁₅H₂₃N₃O₅ required: 325, found: 326 (M+H)⁺.

### Step 2: tert-Butyl 4-(3-acetyl-1,2,4-oxadiazol-5-yl)piperidine-1-carboxylate (M2)

A solution of (M1) in THF (0.15 M) at −78° C. was treated with CH₃MgBr (2 eq.). The reaction mixture was stirred at −78° C. for 1 h, then it was quenched by careful addition of MeOH and diluted with EtOAc. The organic solution was washed with sat. sol. NaHCO₃ and dried (Na₂SO₄). Evaporation of the solvent under reduced pressure gave a crude that was purified by flash chromatography on silica using a gradient of EtOAc/Petroleum ether (1:1) to afford (55%) the title compound. MS (ES⁺) C₁₄H₂₁N₃O₄ required: 295, found: 296 (M+H)⁺.

### Step 3: 2-Fluoro-6-nitrobenzaldehyde (M3)

A mixture of 1-fluoro-2-methyl-3-nitrobenzene and N,N-dimethylformamide dimethyl acetal (2.75 eq.) was heated to 135° C. for 12 h. The reaction mixture was cooled to RT and added dropwise to a solution of NaIO₄ (2.75 eq.) in water/DMF (1:1, 1.1 M). After the addition is finished, the reaction mixture was stirred at RT for 3 h. Then, it was filtered, and the solid was washed with toluene. The filtrate was transferred to a separatory funnel, and the layers were separated. The organic layer was washed with water and dried (Na₂SO₄). Evaporation of the solvent under reduced pressure gave a crude that was purified by flash chromatography on silica using a gradient of EtOAc/Petroleum ether (1:30) to afford (23%) the title compound. MS (ES⁺) C₇H₄FNO₃ required: 169, found: 170 (M+H)⁺.

### Step 4: tert-Butyl 4-[3-(5-fluoroquinolin-2-yl)-1,2,4-oxadiazol-5-yl]piperidine-1-carboxylate (M4)

To a solution of (M3) in EtOH (0.15 M) was added 0.1N HCl (0.05 eq.) and Fe (4 eq.). The reaction mixture was heated to 80° C. for 2 h. After cooling down, the precipitate was filtered off and the filtrate was concentrated to get a crude that was diluted in EtOH. The resulting solution (0.15 M) was treated with KOH (1.2 eq.) and (M2) (1 eq.). The reaction mixture was stirred at RT for 1.5 h. Evaporation of the solvent under reduced pressure gave a crude that was purified by flash chromatography on silica using a gradient of EtOAc/Petroleum ether (1:3) to afford (55%) the title compound. MS (ES⁺) C₂₁H₂₃FN₄O₃ required: 398, found: 399 (M+H)⁺.

### Step 5: 5-Fluoro-2-[5-(piperidinium-4-yl)-1,2,4-oxadiazol-3-yl]quinolinium dichloride (M5)

A solution of (M4) in MeOH (0.25 M), previously saturated with HCl (gas), was stirred at RT for 2 h. Then, the reaction mixture was concentrated and the residue was triturated with Et₂O and the resulting solid filtered to yield (100%) the title compound. MS (ES⁺) C₁₆H₁₅FN₄O required: 298, found: 299 (M+H)⁺.

### Step 6: 4-[3-(5-Fluoroquinolin-2-yl)-1,2,4-oxadiazol-5-yl]-N-{[1-(piperidin-1-yl)cyclopentyl]methyl}piperidine-1-carboxamide (M6)

(M6) was prepared from (M5) following the general procedure reported in Example 91 using 1-[1-(ammoniomethyl)cyclopentyl]piperidinium dichloride as amine. The crude obtained was purified by preparative TLC to afford (24%) the title compound. ¹H NMR (400 MHz, CD₃OD, 300 K) δ 8.70 (1H, d, J=9.2 Hz), 8.30 (1H, d, J=8.4 Hz), 8.05 (1H, d, J=8.4 Hz), 7.85-7.79 (1H, m), 7.42 (1H, t, J=8.0 Hz), 4.13-4.09 (2H, m), 3.61-3.58 (2H, m), 3.49-3.44 (3H, m), 3.29-3.09 (4H, m), 2.25-2.21 (2H, m), 2.09-1.67 (15H, m), 1.60-1.49 (1H, m). MS (ES⁺) C₂₈H₃₅FN₆O₂ required: 506, found: 507 (M+H)⁺.

### EXAMPLE 93

### 6-Chloro-2-(5-{4-[(4,4-difluorocyclohexyl)carbamoyl]piperazin-1-yl}-1,2,4-oxadiazol-3-yl)quinolinium trifluoroacetate (N8)

### Step 1: 6-Chloroquinoline 1-oxide (N1)

A solution of 6-chloroquinoline in DCM (0.3 M) at 0° C. was treated with m-CPBA (1.2 eq.). The reaction mixture was stirred at RT for 4 h. The reaction mixture was diluted with DCM and washed with 1N NaOH and dried (Na₂SO₄). Evaporation of the solvent under reduced pressure afforded (94%) the title compound. MS (ES⁺) C₉H₆ClNO required: 179, 181, found: 180, 182 (M+H)⁺.

### Step 2: 6-Chloroquinoline-2-carbonitrile (N2)

To a solution of compound (N1) in DCM (0.14 M) at 0° C., trimethylsilyl cyanide (5.5 eq.) was added. After stirring for 10 min at the same temperature dimethylcarbamoyl chloride (5.5 eq.) was added. Then, the reaction mixture was stirred at RT for 2 days. After quenching the reaction with 2N NaOH, it was extracted DCM. The combined organic phase was washed with water and dried (Na₂SO₄). Evaporation of the solvent under reduced pressure gave a crude that was purified by flash chromatography on silica using a gradient of EtOAc/Petroleum ether (1:4) to afford (58%) the title compound. MS (ES⁺) C₉H₆ClN₂ required: 188, 190, found: 189, 191 (M+H)⁺.

### Step 3: 6-Chloro-N′-hydroxyquinoline-2-carboximidamide (N3)

A solution of (N2) in EtOH/water (2:1, 0.4 M) was treated with NH₂OH.HCl (1 eq.) and NaHCO₃ (2 eq.). The reaction mixture was heated to reflux for 12 h. After cooling down, the solvent was removed by reduced pressure. Water was added to the resulting residue and after stirring for 30 min, the solid was filtered and dried to afford (93%) the title compound as a yellow powder. ¹H NMR (300 MHz, DMSO-d₆, 300 K) δ 10.29 (1H, s), 8.35 (1H, d, J=9.0 Hz), 8.17 (1H, d, J=2.1 Hz), 8.08 (2H, dd, J=8.4 Hz), 7.83 (1H, dd, J=9.0 and 2.4 Hz), 6.03 (2H, s). MS (ES⁺) C₁₀H₈ClN₃O required: 221, 223, found: 222, 224 (M+H)⁺.

### Step 4: 3-(6-Chloroquinolin-2-yl)-1,2,4-oxadiazol-5-ol (N4)

To a solution of (N3) in DMSO (0.5 M), CDI (2 eq.) and DBU (2 eq.) were added. The reaction mixture was stirred at RT for 3 h. Then, it was poured into 1N HCl and adjusted to pH=6. The resulting precipitate was collected by filtration, washed with water and dried under vacuum to afford (98%) the title compound. ¹H NMR (300 MHz, DMSO-d₆, 300 K) δ 8.63 (1H, d, J=8.7 Hz), 8.32 (1H, d, J=2.4 Hz), 8.16 (2H, dd, J=12.0 and 9.0 Hz), 7.94 (1H, dd, J=9.0 and 2.4 Hz). MS (ES⁺) C₁₁H₆ClN₃O₂ required: 247, found: 248 (M+H)⁺.

### Step 5: 6-Chloro-2-(5-chloro-1,2,4-oxadiazol-3-yl)quinoline (N5)

A solution of (N4) in POCl₃ (0.4 M) was heated to 190° C. for 1 h under microwave irradiation. Then, the reaction mixture was poured into ice-water and adjusted to pH=8.0 with sat. sol. NaHCO₃. The aqueous phase was extracted with DCM and the combined organic phase the was washed with brine and dried (Na₂SO₄). Evaporation of the solvent under reduced pressure gave the title product as a crude that was used directly in the next step without further purification. ¹H NMR (300 MHz, CDCl₃, 300 K) δ 8.26-8.19 (3H, m), 7.87 (1H, d, J=2.4 Hz), 7.72 (1H, dd, J=9.0 and 2.4 Hz). MS (ES⁺) C_{H}H₅Cl₂N₃O₂ required: 265, 267, found: 266, 268 [(M+H)⁺+2]/(M+H)⁺.

### Step 6: tert-Butyl 4-[3-(6-chloroquinolin-2-yl)-1,2,4-oxadiazol-5-yl]piperazine-1-carboxylate (N6)

To a solution of (N5) in THF (0.15 M), tert-butyl 1-piperazinecarboxylate (1.5 eq.) and TEA (2 eq.) were added. The reaction mixture was stirred at RT for 3 h. Then, evaporation of the solvent under reduced pressure gave a crude which was purified by flash chromatography on silica using EtOAc/Petroleum ether (1:8) as solvent to afford (17% over two steps) the title compound. ¹H NMR (300 MHz, CDCl₃, 300 K) δ 8.24 (1H, d, J=9.3 Hz), 8.17 (2H, d, J=3.9 Hz), 7.84 (1H, d, J=2.1 Hz), 7.69 (1H, dd, J=9.0 and 2.1 Hz), 3.78-3.74 (4H, m), 3.61-3.57 (4H, m) 1.50 (9H, s). MS (ES⁺) C₂₀H₂₂ClN₅O₃ required: 415, 417, found: 416, 418 (M+H)⁺.

### Step 7: 6-Chloro-2-[5-(piperazin-4-ium-1-yl)-1,2,4-oxadiazol-3-yl]quinolinium dichloride (N7)

(N7) was prepared from (N6) following the general procedure reported in Example 92 step 5 affording (99%) the title compound. ¹H NMR (300 MHz, DMSO-d₆, 300 K) δ 8.56 (1H, d, J=8.4 Hz), 8.26 (1H, d, J=2.4 Hz), 8.17 (2H, dd, J=12.0 and 9.0 Hz), 7.90 (1H, dd, J=9.0 and 2.4 Hz), 3.63-3.60 (4H, m), 3.40 (1H, s) 2.89-2.85 (4H, m). MS (ES⁺) C₁₅H₁₄ClN₅O required: 315, 317, found: 316, 318 (M+H)⁺.

### Step 8: 6-Chloro-2-(5-{4-[(4,4-difluorocyclohexyl)carbamoyl]piperazin-1-yl}-1,2,4-oxadiazol-3-yl)quinolinium trifluoroacetate (N8)

(N8) was prepared from (N7) following the general procedure reported in Example 91 affording (51%) the title compound. ¹H NMR (400 MHz, CDCl₃, 300 K) δ 8.31-8.27 (2H, m), 8.22 (1H, d, J=8.4 Hz), 7.91 (1H, s), 7.77 (1H, d, J=8.4 Hz), 5.97 (1H, s), 3.84-3.82 (4H, m), 3.61-3.59 (4H, m), 2.12-2.05 (4H, m), 1.98-1.82 (2H, m), 1.59-1.51 (2H, m). MS (ES⁺) C₂₂H₂₃ClF₂N₆O₂ required: 476, 478, found: 477, 479 (M+H)⁺

### EXAMPLE 94

### N-Cyclohexyl-4-[3-(1-methyl-1H-benzimidazol-2-yl)-1,2,4-oxadiazol-5-yl]piperazine-1-carboxamide (O5)

### Step 1: 2-[5-(Trichloromethyl)-1,2,4-oxadiazol-3-yl]-1H-benzimidazole (O1)

Trichloroacetyl chloride (3 eq.) was added dropwise to a mixture of N′-hydroxy-1H-benzimidazole-2-carboximidamide and trichloroacetic acid (3.5 eq.) heated to 85° C. The reaction mixture was heated to 95° C. overnight. After cooling down, EtOAc and iced sat. sol. NaHCO₃ were added and the mixture was stirred at RT until complete neutralization. The organic phase was separated, washed with brine and dried (Na₂SO₄). Evaporation of the solvent under reduced pressure afforded (86%) title compound as beige solid. ¹H NMR (300 MHz, DMSO-d₆, 300 K) δ 7.75-7.64 (2H, m), 7.37-7.28 (2H, m), MS (ES⁺) C₁₀H₅Cl₃N₄O required: 302, 304, found: 303, 305 (M+H)⁺.

### Step 2: tert-Butyl 4-[3-(1H-benzimidazol-2-yl)-1,2,4-oxadiazol-5-yl]piperazine-1-carboxylate (O2)

(O2) was prepared from (O1) following the general procedure reported in Example 11 step 2. Evaporation of the solvent under reduced pressure afforded the crude title compound as a yellow oil that was used as such in the next step. ¹H NMR (300 MHz, DMSO-d₆, 300 K) δ 7.73-7.54 (2H, m), 7.34-7.20 (2H, m), 3.71-3.59 (4H, m), 3.56-3.47 (4H, m), 1.44 (9H, s). MS (ES⁺) C₁₈H₂₂N₆O₃ required: 370, found: 371 (M+H)⁺.

### Step 3: tert-Butyl 4-[3-(1-methyl-1H-benzimidazol-2-yl)-1,2,4-oxadiazol-5-yl]piperazine-1-carboxylate (O3)

A solution of (O2) in acetone (0.2 M) at RT was treated, first, with powdered KOH (16 eq.) and, after a few minutes, with methyl iodide (19 eq.). The reaction mixture was stirred at RT overnight. Then, solvent was evaporated to dryness and the resulting residue was partitioned between DCM and brine. The organic phase was separated and dried (Na₂SO₄). Evaporation of the solvent under reduced pressure gave a residue that was purified by flash chromatography on silica using a gradient of EtOAc/Petroleum ether from 12% to 100% EtOAc to afford (33% over two steps) the title compound as a white solid. ¹H NMR (300 MHz, DMSO-d₆, 300 K) δ 7.75 (1H, d, J=8.0 Hz), 7.69 (1H, d, J=8.0 Hz), 7.44-7.36 (1H, m), 7.35-7.27 (1H, m), 4.09 (3H, s), 3.69-3.60 (4H, m), 3.56-3.47 (4H, m), 1.44 (9H, s). MS (ES⁺) C₁₉H₂₄N₆O₃ required: 384, found: 385 (M+H)⁺.

### Step 4: 1-Methyl-2-(5-piperazin-4-ium-1-yl-1,2,4-oxadiazol-3-yl)-1H-benzimidazol-1-ium bis(trifluoroacetate) (O4)

(O4) was prepared from (O3) following the general procedure reported in Example 11 step 3 affording (100%) the title compound as a beige powder. ¹H NMR (300 MHz, CD₄OD, 300 K) δ 7.85-7.82 (1H, m), 7.81-7.79 (1H, m), 7.64-7.50 (2H, m), 4.29 (3H, s), 4.07-3.99 (4H, m), 3.48-3.41 (4H, m). MS (ES⁺) C₁₄H₁₆N₆O required: 284, found: 285 (M+H)⁺.

### Step 5: N-Cyclohexyl-4-[3-(1-methyl-1H-benzimidazol-2-yl)-1,2,4-oxadiazol-5-yl]piperazine-1-carboxamide (O5)

(O5) was prepared from (O4) following the general procedure reported in Example 2 step 2 using isocyanatocyclohexane (1 eq.). The crude was purified by flash chromatography on silica using a gradient of MeOH/DCM from 1 to 5% MeOH to afford (71%) the title compound as a white powder. ¹H NMR (300 MHz, DMSO-d₆, 300 K) δ 7.75 (1H, d, J=8.0 Hz), 7.69 (1H, d, J=8.0 Hz), 7.45-7.36 (1H, m), 7.35-7.27 (1H, m), 6.37-6.29 (1H, m), 4.10 (3H, s), 3.67-3.56 (4H, m), 3.53-3.45 (4H, m), 3.44-3.37 (1H, m), 1.83-1.64 (4H, m), 1.63-1.50 (1H, m), 1.34-1.02 (5H, m). MS (ES⁺) C₂₁H₂₇N₇O₂ required: 409, found: 410 (M+H)⁺.

### EXAMPLE 95

### 2-(5-{4-[Cyclohexylmethyl)carbamoyl]piperazin-1-yl}-1,2,4-oxadiazol-3-yl)-1-methyl-1H-3,1-benzimidazol-3-ium trifluoroacetate (P1)

A solution of N-methylcyclohexanamine (1.5 eq.) and TEA (2.1 eq.) in DCM (0.25 M) was added dropwise to a solution of phosgene (1.8 eq., 20% solution in toluene) in DCM (0.25 M) at 0° C. The reaction mixture was stirred at 0° C. for 1 h. Then, it was diluted with DCM and washed with 1N HCl, brine and dried (Na₂SO₄). Evaporation of the solvent gave the crude carbamoyl chloride as a yellow oil, which was dissolved in DCM (0.5 M) and added to a solution of (O4) and TEA (3.5 eq.) in DMF at 0° C. The resulting mixture was stirred at RT overnight. After evaporation of the solvent the product was directly purified by preparative RP-HPLC using water (+0.1% TFA) and MeCN (+0.1% TFA) as eluents (column: C₁₈). The product fractions were lyophilized to afford (65%) the title compound as a white powder. ¹H NMR (300 MHz, DMSO-d₆, 300 K) δ 7.79-7.67 (2H, m), 7.46-7.37 (1H, m), 7.36-7.29 (1H, m), 4.10 (3H, s), 3.58 (4H, m), 3.57-3.42 (1H, m), 3.29-3.17 (4H, m), 2.71 (3H, s), 1.81-1.69 (2H, m), 1.67-1.54 (3H, m), 1.53-1.36 (2H, m), 1.35-1.17 (2H, m), 1.15-0.97 (1H, m). MS (ES⁺) C₂₂H₂₉N₇O₂ required: 423, found: 424 (M+H)⁺.

### EXAMPLE 96

### 2-(5-{4-[(4,4-Difluorocyclohexyl)carbamoyl]piperazin-1-yl}-1,2,4-oxadiazol-3-yl)-6-fluoro-1-methyl-1H-3,1-benzimidazol-3-ium trifluoroacetate (Q10)

### Step 1: 4-Fluorobenzene-1,2-diamine (Q1)

A solution of 5-fluoro-2-nitroaniline in MeOH (0.32 M) was added to Pd/C (5% w/w, 10% w). The reaction mixture was stirred at R.T. under H₂ atmosphere (50 Psi) for 2 h. Then, the mixture was filtrated and the organic solution was concentrated to afford (90%) the title compound as a brown oil. MS (ES⁺) C₆H₇FN₂ required: 126, found: 127 (M+H)⁺.

### Step 2: 6-Fluoro-2-(trichloromethyl)-1H-benzimidazole (Q2)

Cl₃CC(NH)OMe (1 eq.) was added slowly to a solution of compound (Q1) in AcOH (0.8 M) at 0° C., then the reaction mixture was stirred at RT for 2 h. The reaction mixture was diluted with EtOAc, and the resulting organic phase was washed The with sat. aq. NaHCO₃ and dried (Na₂SO₄). Evaporation of the solvent under reduced pressure afforded (75%) the title compound. MS (ES⁺) C₈H₄Cl₃FN₂ required: 252, 254, found: 253, 255 (M+H)⁺.

### Step 3: 6-Fluoro-1H-benzimidazole-2-carbonitrile (Q3)

NH₃ (exc.) was added to a round bottom flask containing (Q2) and cooled to −78° C. The reaction mixture was allowed to warm to R.T. and ammonia was evaporated affording (90%) the title compound. MS (ES⁺) C₈H₄FN₃ required: 161, found: 162 (M+H)⁺.

### Step 4: 6-Fluoro-1-methyl-1H-benzimidazole-2-carbonitrile (Q4) and 5-fluoro-1-methyl-1H-benzimidazole-2-carbonitrile (Q5)

(MeO)₂SO₂ (1 eq.) was added dropwise to a solution of (Q3), NaOH (1.5 eq.) and TBAB (0.1 eq.) in DMSO (0.12 M). The mixture was stirred at RT overnight. Then, the reaction mixture was diluted with water and extracted with EtOAc. The combined organic phase was dried (Na₂SO₄) and evaporation of the solvent under reduced pressure gave a residue that was purified by flash chromatography on silica using a gradient of EtOAc/Petroleum ether (1:3) to afford (38%) 6-fluoro-1-methyl-1H-benzimidazole-2-carbonitrile as a white solid. MS (ES⁺) C₉H₆FN₃ required: 175, found: 176 (M+H)⁺. The same chromatography separation afforded (37%) 5-fluoro-1-methyl-1H-benzimidazole-2-carbonitrile as a white solid. MS (ES⁺) C₉H₆FN₃ required: 175, found: 176 (M+H)⁺.

### Step 5: 6-Fluoro-N′-hydroxy-1-methyl-1H-benzimidazole-2-carboximidamide (Q6)

A solution of (Q5) in MeOH (0.25 M) was treated with a solution of NH₂OH in water (50% w/w, 1.1 eq.). The mixture was heated to 110° C. for 40 min under microwave irradiation. Then, the solvent was removed by reduced pressure. The resulting residue was dissolved in DCM and the organic phase was washed with water and dried (Na₂SO₄). Evaporation of the solvent under reduced pressure gave a crude which was purified by flash chromatography on silica using EtOAc/Petroleum ether as solvent to afford (90%) the title compound. MS (ES⁺) C₉H₉FN₄O required: 208, found: 209 (M+H)⁺.

### Step 6: 6-Fluoro-1-methyl-2-[5-(trichloromethyl)-1,2,4-oxadiazol-3-yl]-1H-benzimidazole (Q⁷)

(Q7) was prepared from (Q6) following the general procedure reported in Example 94 step 1 affording (78%) the title compound. MS (ES⁺) C₁₁H₆Cl₃FN₄O required: 335, found: 336 (M+H)⁺.

### Step 7: tert-Butyl 4-[3-(6-fluoro-1-methyl-1H-benzimidazol-2-yl)-1,2,4-oxadiazol-5-yl]piperazine-1-carboxylate (Q8)

(Q8) was prepared from (Q7) and DIPEA (2 eq.) following the general procedure reported in Example 11 step 2. The reaction crude was purified by flash chromatography on silica using EtOAc/Petroleum ether (1:4) as solvent to afford (62%) the title compound. MS (ES⁺) C₁₉H₂₃FN₆O₃ required: 402, found: 403 (M+H)⁺.

### Step 8: 6-Fluoro-1-methyl-2-[5-(piperazin-4-ium-1-yl)-1,2,4-oxadiazol-3-yl]-1H-3,1-benzimidazol-3-ium dichloride (Q9)

(Q9) was prepared from (Q8) following the general procedure reported in Example 92 step 5 affording (96%) the title product. MS (ES⁺) C₁₄H₁₅FN₆O required: 302, found: 303 (M+H)⁺.

### Step 9: 2-(5-{4-[(4,4-Difluorocyclohexyl)carbamoyl]piperazin-1-yl}-1,2,4-oxadiazol-3-yl)-6-fluoro-1-methyl-1H-3,1-benzimidazol-3-ium trifluoroacetate (Q10)

(Q10) was prepared from (Q9) following the general procedure reported in Example 91 affording (40%) the title compound. ¹H NMR (400 MHz, CDCl₃, 300 K) δ 7.94-7.91 (1H, m), 7.25-7.19 (2H, m), 4.20 (3H, s), 3.78-3.68 (5H, m), 3.55-3.53 (4H, m), 2.12-1.99 (4H, m), 1.92-1.74 (2H, m), 1.55-1.47 (2H, m). MS (ES⁺) C₂₁H₂₄F₃N₇O₂ required: 463, found: 464 (M+H)⁺.

### EXAMPLE 97

### 2-(5-{4-[(4,4-Difluorocyclohexyl)carbamoyl]piperazin-1-yl}-1,2,4-oxadiazol-3-yl)-5-fluoro-1-methyl-1H-3,1-benzimidazol-3-ium trifluoroacetate (R5)

### Step 1: 5-Fluoro-N′-hydroxy-1-methyl-1H-benzimidazole-2-carboximidamide (R1)

(R1) was prepared from 5-fluoro-1-methyl-1-H-benzimidazole-2-carbonitrile (see Q5) following the general procedure reported in Example 96 step 5 affording (90%) the title compound. MS (ES⁺) C₉H₉FN₄O required: 208, found: 209 (M+H)⁺.

### Step 2: 5-Fluoro-1-methyl-2-[5-(trichloromethyl)-1,2,4-oxadiazol-3-yl]-1H-benzimidazole (R2)

(R2) was prepared from (R1) following the general procedure reported in Example 96 step 6 affording (83%) the title compound. MS (ES⁺) C₁₁H₆Cl₃FN₄O required: 335, found: 336 (M+H)⁺.

### Step 3: tert-Butyl 4-[3-(5-fluoro-1-methyl-1H-benzimidazol-2-yl)-1,2,4-oxadiazol-5-yl]piperazine-1-carboxylate (R3)

(R3) was prepared from (R2) following the general procedure reported in Example 96 step 7 affording (55%) the title compound. MS (ES⁺) C₁₉H₂₃FN₆O₃ required: 402, found: 403 (M+H)⁺.

### Step 4: 5-Fluoro-1-methyl-2-[5-(piperazin-4-ium-1-yl)-1,2,4-oxadiazol-3-yl]-1H-3,1-benzimidazol-3-ium dichloride (R4)

(R4) was prepared from (R3) following the general procedure reported in Example 92 step 5 affording (94%) the title compound. MS (ES⁺) C₁₄H₁₅FN₆O required: 302, found: 303 (M+H)⁺.

### Step 5: 2-(5-{4-[(4,4-Difluorocyclohexyl)carbamoyl]piperazin-1-yl}-1,2,4-oxadiazol-3-yl)-5-fluoro-1-methyl-1H-3,1-benzimidazol-3-ium trifluoroacetate (R5)

(R5) was prepared from (R4) following the general procedure reported in Example 91 affording (35%) the title compound. ¹H NMR (400 MHz, CDCl₃, 300 K) δ 7.50-7.46 (1H, m), 7.31-7.27 (1H, m), 7.12-7.06 (1H, m), 4.29-4.27 (1H, m), 4.08 (3H, s), 3.75-3.72 (5H, m), 3.48-3.45 (4H, m), 2.07-1.95 (4H, m), 1.80-1.71 (2H, m), 1.49-1.42 (2H, m). MS (ES⁺) C₂ᵢH₂₄F₃N₇O₂ required: 463, found: 464 (M+H)⁺.

The compounds in the following tables were made according to the procedures described above:

TABLE 5
Further examples of ureidopiperidineoxadiazole compounds

| | | Molecular Ion | Procedure of |
|---|---|---|---|
| No. | Name | [M + H]⁺ | Example |
| 98 | 2-(5-{1-[(2,6-dichloropyridin-4- | 469, 471 | 1 |
| | yl)carbamoyl]piperidin-4-yl}-1,2,4-oxadiazol- | | |
| | 3-yl)quinolinium trifluoroacetate | | |
| 99 | 2-(5-{1-[(3,5-dimethylisoxazol-4- | 419 | 1 |
| | yl)carbamoyl]piperidin-4-yl}-1,2,4-oxadiazol- | | |
| | 3-yl)quinolinium trifluoroacetate | | |
| 100 | 2-(5-{1-[(1-methylpiperidinium-4- | 421 | 8 |
| | yl)carbamoyl]piperidin-4-yl}-1,2,4-oxadiazol- | | |
| | 3-yl)quinolinium bis(trifluoroacetate) | | |
| 101 | 2-(5-{1-[(2- | 435, 437 | 1 |
| | chlorophenyl)carbamoyl]piperidin-4-yl}- | | |
| | 1,2,4-oxadiazol-3-yl)-1,6-naphthyridin-1-ium | | |
| | trifluoroacetate | | |
| 102 | 2-(5-{1-[(2-methyl-7-aza-2- | 447 | 8 |
| | azoniaspiro[4.4]non-7-yl)carbonyl]piperidin- | | |
| | 4-yl}-1,2,4-oxadiazol-3-yl)quinolinium | | |
| | bis(trifluoroacetate) | | |
| 103 | 2-[5-(1-{[2-(1H-1,2,4-triazol-4-ium-1- | 419 | 2 |
| | yl)ethyl]carbamoyl}piperidin-4-yl)-1,2,4- | | |
| | oxadiazol-3-yl]quinolinium | | |
| | bis(trifluoroacetate) | | |
| 104 | 2-[5-(1-{[2-(pyrazin-4-ium-2- | 430 | 2 |
| | yl)ethyl]carbamoyl}piperidin-4-yl)-1,2,4- | | |
| | oxadiazol-3-yl]quinolinium | | |
| | bis(trifluoroacetate) | | |
| 105 | 2-[5-(1-{[(1-oxidopyridin-3- | 431 | 8 |
| | yl)methyl]carbamoyl}piperidin-4-yl)-1,2,4- | | |
| | oxadiazol-3-yl]quinolinium trifluoroacetate | | |
| 106 | 2-{5-[1-(8-aza-1-azoniaspiro[4.5]dec-8- | 447 | 8 |
| | ylcarbonyl)piperidin-4-yl]-1,2,4-oxadiazol-3- | | |
| | yl}quinolinium bis(trifluoroacetate) | | |
| 107 | 2-[5-(1-{[(2-oxo-1,2-dihydropyridin-3- | 431 | 8 |
| | yl)methyl]carbamoyl}piperidin-4-yl)-1,2,4- | | |
| | oxadiazol-3-yl]quinolinium trifluoroacetate | | |
| 108 | N-(4-chloropyridin-3-yl)-4-[3-(quinolin-2-yl)- | 435, 437 | 1 |
| | 1,2,4-oxadiazol-5-yl]piperidine-1- | | |
| | carboxamide | | |
| 109 | N-(2-chloropyridin-3-yl)-4-[3-(quinolin-2-yl)- | 435, 437 | 1 |
| | 1,2,4-oxadiazol-5-yl]piperidine-1- | | |
| | carboxamide | | |
| 110 | 2-(5-{1-[ethyl(propan-2- | 358 | 3 |
| | yl)carbamoyl]piperidin-4-yl}-1,2,4-oxadiazol- | | |
| | 3-yl)-3-methylpyridinium trifluoroacetate | | |
| 111 | 6-[({4-[3-(quinolinium-2-yl)-1,2,4-oxadiazol- | 452 | 1 |
| | 5-yl]piperidin-1- | | |
| | yl}carbonyl)amino]quinoxalin-1-ium | | |
| | bis(trifluoroacetate) | | |
| 112 | 2-{5-[1-(quinolinium-3- | 451 | 1 |
| | ylcarbamoyl)piperidin-4-yl]-1,2,4-oxadiazol- | | |
| | 3-yl}quinolinium bis(trifluoroacetate) | | |
| 113 | 2-(5-{1-[(1-methyl-1H-indol-5- | 453 | 1 |
| | yl)carbamoyl]piperidin-4-yl}-1,2,4-oxadiazol- | | |
| | 3-yl)quinolinium trifluoroacetate | | |
| 114 | 2-(5-{1-[(3-chloro-5- | 459, 461 | 1 |
| | cyanophenyl)carbamoyl]piperidin-4-yl}- | | |
| | 1,2,4-oxadiazol-3-yl)quinolinium | | |
| | trifluoroacetate | | |
| 115 | 2-(5-{1-[(2-methyl-1,3-benzoxazol-6- | 455 | 1 |
| | yl)carbamoyl]piperidin-4-yl}-1,2,4-oxadiazol- | | |
| | 3-yl)quinolinium trifluoroacetate | | |
| 116 | 2-{5-[1-(quinolinium-6- | 451 | 1 |
| | ylcarbamoyl)piperidin-4-yl]-1,2,4-oxadiazol- | | |
| | 3-yl}quinolinium bis(trifluoroacetate) | | |
| 117 | 2-{5-[1-(1,3-benzothiazol-5- | 457 | 1 |
| | ylcarbamoyl)piperidin-4-yl]-1,2,4-oxadiazol- | | |
| | 3-yl}quinolinium trifluoroacetate | | |
| 118 | 2-(5-{1-[(1-acetylpiperidin-4- | 449 | 8 |
| | yl)carbamoyl]piperidin-4-yl}-1,2,4-oxadiazol- | | |
| | 3-yl)quinolinium trifluoroacetate | | |
| 119 | 2-[5-(1-{[2-chloro-5- | 512, 514 | 1 |
| | (methylsulfonyl)phenyl]carbamoyl}piperidin- | | |
| | 4-yl)-1,2,4-oxadiazol-3-yl]quinolinium | | |
| | trifluoroacetate | | |
| 120 | 2-{5-[1-(2,3-dihydro-1,4-benzodioxin-5- | 458 | 1 |
| | ylcarbamoyl)piperidin-4-yl]-1,2,4-oxadiazol- | | |
| | 3-yl}quinolinium trifluoroacetate | | |
| 121 | 2-[5-(1-{[2-chloro-4- | 512, 514 | 1 |
| | (methylsulfonyl)phenyl]carbamoyl}piperidin- | | |
| | 4-yl)-1,2,4-oxadiazol-3-yl]quinolinium | | |
| | trifluoroacetate | | |
| 122 | 2-(5-{1-[(2- | 425 | 1 |
| | cyanophenyl)carbamoyl]piperidin-4-yl}- | | |
| | 1,2,4-oxadiazol-3-yl)quinolinium | | |
| | trifluoroacetate | | |
| 123 | 2-(5-{1-[(2-chloro-5- | 459 | 1 |
| | cyanophenyl)carbamoyl]piperidin-4-yl}- | | |
| | 1,2,4-oxadiazol-3-yl)quinolinium | | |
| | trifluoroacetate | | |
| 124 | 2-(5-{1-[(6-cyanopyridinium-3- | 426 | 1 |
| | yl)carbamoyl]piperidin-4-yl}-1,2,4-oxadiazol- | | |
| | 3-yl)quinolinium bis(trifluoroacetate) | | |
| 125 | 2-(5-{1-[(1,1-dioxidotetrahydro-2H- | 420 | 3 |
| | thiopyran-4-yl)carbamoyl]piperidin-4-yl}- | | |
| | 1,2,4-oxadiazol-3-yl)-3-methylpyridinium | | |
| | trifluoroacetate | | |
| 126 | 2-[5-(1-{[2-(1,3-oxazol-5- | 467 | 1 |
| | yl)phenyl]carbamoyl}piperidin-4-yl)-1,2,4- | | |
| | oxadiazol-3-yl]quinolinium trifluoroacetate | | |
| 127 | 2-(5-{1-[(2-chloro-4- | 459, 461 | 1 |
| | cyanophenyl)carbamoyl]piperidin-4-yl}- | | |
| | 1,2,4-oxadiazol-3-yl)quinolinium | | |
| | trifluoroacetate | | |
| 128 | N-(2-chlorophenyl)-4-[3-(6-methoxyquinolin- | 464, 466 | 1 |
| | 2-yl)-1,2,4-oxadiazol-5-yl]piperidine-1- | | |
| | carboxamide | | |
| 129 | N-cyclohexyl-4-[3-(6-methoxyquinolin-2-yl)- | 436 | 2 |
| | 1,2,4-oxadiazol-5-yl]piperidine-1- | | |
| | carboxamide | | |
| 130 | 2-[5-(1-{[(2-methyltetrahydrofuran-2- | 422 | 8 |
| | yl)methyl]carbamoyl}piperidin-4-yl)-1,2,4- | | |
| | oxadiazol-3-yl]quinolinium trifluoroacetate | | |
| 131 | 2-(5-{1-[(5-fluoro-2- | 396 | 3 |
| | methylphenyl)carbamoyl]piperidin-4-yl}- | | |
| | 1,2,4-oxadiazol-3-yl)-3-methylpyridinium | | |
| | trifluoroacetate | | |
| 132 | 2-{5-[1-(1-benzofuran-5- | 404 | 3 |
| | ylcarbamoyl)piperidin-4-yl]-1,2,4-oxadiazol- | | |
| | 3-yl}-3-methylpyridinium trifluoroacetate | | |
| 133 | 2-(5-{1-[(2,3- | 432, 434 | 3 |
| | dichlorophenyl)carbamoyl]piperidin-4-yl}- | | |
| | 1,2,4-oxadiazol-3-yl)-3-methylpyridinium | | |
| | trifluoroacetate | | |
| 134 | 2-(5-{1-[(3- | 389 | 3 |
| | cyanophenyl)carbamoyl]piperidin-4-yl}- | | |
| | 1,2,4-oxadiazol-3-yl)-3-methylpyridinium | | |
| | trifluoroacetate | | |
| 135 | 2-{5-[1-({[1- | 436 | 8 |
| | (hydroxymethyl)cyclopentyl]methyl}carbamoyl)piperidin- | | |
| | 4-yl]-1,2,4-oxadiazol-3- | | |
| | yl}quinolinium trifluoroacetate | | |
| 136 | 2-[5-(1-{[(4-methyl-1,3-thiazol-2- | 435 | 2 |
| | yl)methyl]carbamoyl}piperidin-4-yl)-1,2,4- | | |
| | oxadiazol-3-yl]quinolinium trifluoroacetate | | |
| 137 | 3-methyl-2-{5-[1-(naphthalen-2- | 414 | 3 |
| | ylcarbamoyl)piperidin-4-yl]-1,2,4-oxadiazol- | | |
| | 3-yl}pyridinium trifluoroacetate | | |
| 138 | 2-(5-{1-[(2- | 435, 437 | 1 |
| | chlorophenyl)carbamoyl]piperidin-4-yl}- | | |
| | 1,2,4-oxadiazol-3-yl)quinoxalin-1-ium | | |
| | trifluoroacetate | | |
| 139 | 2-(5-{1-[methyl(tetrahydro-2H-pyran-4- | 436 | 8 |
| | ylmethyl)carbamoyl]piperidin-4-yl}-1,2,4- | | |
| | oxadiazol-3-yl)quinolinium trifluoroacetate | | |
| 140 | 2-{5-[1-(cyclopentylcarbamoyl)piperidin-4- | 392 | 2 |
| | yl]-1,2,4-oxadiazol-3-yl}quinolinium | | |
| | trifluoroacetate | | |
| 141 | 2-(5-{1-[(4- | 422 | 8 |
| | hydroxycyclohexyl)carbamoyl]piperidin-4- | | |
| | yl}-1,2,4-oxadiazol-3-yl)quinolinium | | |
| | trifluoroacetate | | |
| 142 | 2-[5-(1-{[2-(pyridinium-4- | 477 | 1 |
| | yl)phenyl]carbamoyl}piperidin-4-yl)-1,2,4- | | |
| | oxadiazol-3-yl]quinolinium | | |
| | bis(trifluoroacetate) | | |
| 143 | 2-(5-{1-[(2-chloro-5- | 513, 515 | 1 |
| | sulfamoylphenyl)carbamoyl]piperidin-4-yl}- | | |
| | 1,2,4-oxadiazol-3-yl)quinolinium | | |
| | trifluoroacetate | | |
| 144 | 2-(5-{1-[(1,1-dioxidotetrahydro-2H- | 486 | 8 |
| | thiopyran-4-yl)carbamoyl]piperidin-4-yl}- | | |
| | 1,2,4-oxadiazol-3-yl)-6-methoxyquinolinium | | |
| | trifluoroacetate | | |
| 145 | 2-{5-[1-(1,3-benzoxazol-4- | 441 | 1 |
| | ylcarbamoyl)piperidin-4-yl]-1,2,4-oxadiazol- | | |
| | 3-yl}quinolinium trifluoroacetate | | |
| 146 | 2-[5-(1-{[4- | 507 | 1 |
| | (dimethylsulfamoyl)phenyl]carbamoyl}piperidin- | | |
| | 4-yl)-1,2,4-oxadiazol-3-yl]quinolinium | | |
| | trifluoroacetate | | |
| 147 | N-(quinolin-8-yl)-4-[3-(quinolin-2-yl)-1,2,4- | 451 | 1 |
| | oxadiazol-5-yl]piperidine-1-carboxamide | | |
| 148 | 2-{5-[1-(piperidinium-4- | 407 | 8 |
| | ylcarbamoyl)piperidin-4-yl]-1,2,4-oxadiazol- | | |
| | 3-yl}quinolinium bis(trifluoroacetate) | | |
| 149 | 2-(5-{1-[(3-methyl-1,1- | 456 | 8 |
| | dioxidotetrahydrothiophen-3- | | |
| | yl)carbamoyl]piperidin-4-yl}-1,2,4-oxadiazol- | | |
| | 3-yl)quinolinium trifluoroacetate | | |
| 150 | N-(1,1-dioxidotetrahydrothiophen-3-yl)-4-[3- | 442 | 8 |
| | (quinolin-2-yl)-1,2,4-oxadiazol-5- | | |
| | yl]piperidine-1-carboxamide | | |
| 151 | 2-(5-{1-[(1-methyl-1H-3,1-benzimidazol-3- | 454 | 1 |
| | ium-5-yl)carbamoyl]piperidin-4-yl}-1,2,4- | | |
| | oxadiazol-3-yl)quinolinium | | |
| | bis(trifluoroacetate) | | |
| 152 | 2-(5-{1-[(2-chloro-4- | 513, 515 | 1 |
| | sulfamoylphenyl)carbamoyl]piperidin-4-yl}- | | |
| | 1,2,4-oxadiazol-3-yl)quinolinium | | |
| | trifluoroacetate | | |
| 153 | 6-chloro-2-(5-{1-[(2- | 468, 470 | 1 |
| | chlorophenyl)carbamoyl]piperidin-4-yl}- | | |
| | 1,2,4-oxadiazol-3-yl)quinolinium | | |
| 154 | 6-chloro-2-(5-{1-[(1,1-dioxidotetrahydro-2H- | 490, 492 | 8 |
| | thiopyran-4-yl)carbamoyl]piperidin-4-yl}- | | |
| | 1,2,4-oxadiazol-3-yl)quinolinium | | |
| | trifluoroacetate | | |
| 155 | 6-chloro-2-{5-[1- | 440, 442 | 2 |
| | (cyclohexylcarbamoyl)piperidin-4-yl]-1,2,4- | | |
| | oxadiazol-3-yl}quinolinium trifluoroacetate | | |
| 156 | 2-{5-[1-(tetrahydro-2H-thiopyran-4- | 424 | 8 |
| | ylcarbamoyl)piperidin-4-yl]-1,2,4-oxadiazol- | | |
| | 3-yl}quinolinium trifluoroacetate | | |
| 157 | 2-[5-(1-{[4- | 478 | 1 |
| | (methylsulfonyl)phenyl]carbamoyl}piperidin- | | |
| | 4-yl)-1,2,4-oxadiazol-3-yl]quinolinium | | |
| | trifluoroacetate | | |
| 158 | 4-[3-(quinolin-2-yl)-1,2,4-oxadiazol-5-yl]-N- | 422 | 8 |
| | (tetrahydro-2H-pyran-2-ylmethyl)piperidine- | | |
| | 1-carboxamide | | |
| 159 | 4-[3-(quinolin-2-yl)-1,2,4-oxadiazol-5-yl]-N- | 408 | 8 |
| | (tetrahydrofuran-3-ylmethyl)piperidine-1- | | |
| | carboxamide | | |
| 160 | (3,3-difluoropiperidin-1-yl){4-[3-(quinolin-2- | 428 | 8 |
| | yl)-1,2,4-oxadiazol-5-yl]piperidin-1- | | |
| | yl}methanone | | |
| 161 | N-(1-methylcyclohexyl)-4-[3-(quinolin-2-yl)- | 420 | 8 |
| | 1,2,4-oxadiazol-5-yl]piperidine-1- | | |
| | carboxamide | | |
| 162 | N-(1-methyl-2-oxopyrrolidin-3-yl)-4-[3- | 421 | 8 |
| | (quinolin-2-yl)-1,2,4-oxadiazol-5- | | |
| | yl]piperidine-1-carboxamide | | |
| 163 | N-(1,1-dioxidotetrahydrothiophen-3-yl)-N- | 456 | 8 |
| | methyl-4-[3-(quinolin-2-yl)-1,2,4-oxadiazol- | | |
| | 5-yl]piperidine-1-carboxamide | | |
| 164 | 4-(1-{[({4-[3-(1,5-naphthyridin-2-yl)-1,2,4- | 492 | 8 |
| | oxadiazol-5-yl]piperidin-1- | | |
| | yl}carbonyl)amino]methyl}cyclopentyl)morpholin- | | |
| | 4-ium trifluoroacetate | | |
| 165 | 4-(1-{[({4-[3-(quinolin-2-yl)-1,2,4-oxadiazol- | 505 | 8 |
| | 5-yl]piperidin-1- | | |
| | yl}carbonyl)amino]methyl}cyclohexyl)morpholin- | | |
| | 4-ium trifluoroacetate | | |
| 166 | 4-(1-{[({4-[3-(quinolin-2-yl)-1,2,4-oxadiazol- | 519 | 8 |
| | 5-yl]piperidin-1- | | |
| | yl}carbonyl)amino]methyl}cycloheptyl)morpholin- | | |
| | 4-ium trifluoroacetate | | |
| 167 | 1-(1-{[({4-[3-(quinolin-2-yl)-1,2,4-oxadiazol- | 489 | 8 |
| | 5-yl]piperidin-1- | | |
| | yl}carbonyl)amino]methyl}cyclopentyl)piperidinium | | |
| | trifluoroacetate | | |
| 168 | 1-(1-{[({4-[3-(quinolin-2-yl)-1,2,4-oxadiazol- | 503 | 8 |
| | 5-yl]piperidin-1- | | |
| | yl}carbonyl)amino]methyl}cyclohexyl)piperidinium | | |
| | trifluoroacetate | | |
| 169 | 4-methyl-1-(1-{[({4-[3-(quinolin-2-yl)-1,2,4- | 518 | 8 |
| | oxadiazol-5-yl]piperidin-1- | | |
| | yl}carbonyl)amino]methyl}cyclohexyl)piperazin- | | |
| | 1-ium trifluoroacetate | | |
| 170 | N,N,1-trimethyl-4-{[({4-[3-(quinolin-2-yl)- | 478 | 8 |
| | 1,2,4-oxadiazol-5-yl]piperidin-1- | | |
| | yl}carbonyl)amino]methyl}piperidin-4- | | |
| | aminium trifluoroacetate | | |
| 171 | 1-methyl-4-phenyl-4-{[({4-[3-(quinolin-2-yl)- | 511 | 8 |
| | 1,2,4-oxadiazol-5-yl]piperidin-1- | | |
| | yl}carbonyl)amino]methyl}piperidinium | | |
| | trifluoroacetate | | |
| 172 | 4-{1-(pyridin-2-yl)-2-[({4-[3-(quinolin-2-yl)- | 514 | 8 |
| | 1,2,4-oxadiazol-5-yl]piperidin-1- | | |
| | yl}carbonyl)amino]ethyl}morpholin-4-ium | | |
| | trifluoroacetate | | |
| 173 | 4-{1-(pyridin-3-yl)-2-[({4-[3-(quinolin-2-yl)- | 514 | 8 |
| | 1,2,4-oxadiazol-5-yl]piperidin-1- | | |
| | yl}carbonyl)amino]ethyl}morpholin-4-ium | | |
| | trifluoroacetate | | |
| 174 | 4-{1-(pyridin-4-yl)-2-[({4-[3-(quinolin-2-yl)- | 514 | 8 |
| | 1,2,4-oxadiazol-5-yl]piperidin-1- | | |
| | yl}carbonyl)amino]ethyl}morpholin-4-ium | | |
| | trifluoroacetate | | |
| 175 | 1-{1-phenyl-2-[({4-[3-(quinolin-2-yl)-1,2,4- | 511 | 8 |
| | oxadiazol-5-yl]piperidin-1- | | |
| | yl}carbonyl)amino]ethyl}piperidinium | | |
| | trifluoroacetate | | |
| 176 | 1-{1-(4-fluorophenyl)-2-[({4-[3-(quinolin-2- | 529 | 8 |
| | yl)-1,2,4-oxadiazol-5-yl]piperidin-1- | | |
| | yl}carbonyl)amino]ethyl}piperidinium | | |
| | trifluoroacetate | | |
| 177 | 1-{1-(pyridin-3-yl)-2-[({4-[3-(quinolin-2-yl)- | 512 | 8 |
| | 1,2,4-oxadiazol-5-yl]piperidin-1- | | |
| | yl}carbonyl)amino]ethyl}piperidinium | | |
| | trifluoroacetate | | |
| 178 | N,N-dimethyl-1-phenyl-2-[({4-[3-(quinolin-2- | 471 | 8 |
| | yl)-1,2,4-oxadiazol-5-yl]piperidin-1- | | |
| | yl}carbonyl)amino]ethanaminium | | |
| | trifluoroacetate | | |
| 179 | 1-{1-phenyl-2-[({4-[3-(quinolin-2-yl)-1,2,4- | 497 | 8 |
| | oxadiazol-5-yl]piperidin-1- | | |
| | yl}carbonyl)amino]ethyl}pyrrolidinium | | |
| | trifluoroacetate | | |
| 180 | N,N-dimethyl-1-(pyridin-2-yl)-2-[({4-[3- | 472 | 8 |
| | (quinolin-2-yl)-1,2,4-oxadiazol-5- | | |
| | yl]piperidin-1- | | |
| | yl}carbonyl)amino]ethanaminium | | |
| | trifluoroacetate | | |
| 181 | 4-{2-methyl-1-[({4-[3-(quinolin-2-yl)-1,2,4- | 465 | 8 |
| | oxadiazol-5-yl]piperidin-1- | | |
| | yl}carbonyl)amino]propan-2-yl}morpholin-4- | | |
| | ium trifluoroacetate | | |
| 182 | 1,1,1-trifluoro-N,N-dimethyl-3-[({4-[3- | 463 | 8 |
| | (quinolin-2-yl)-1,2,4-oxadiazol-5- | | |
| | yl]piperidin-1-yl}carbonyl)amino]propan-2- | | |
| | aminium trifluoroacetate | | |
| 183 | 2-{2,2-dimethyl-3-[({4-[3-(quinolin-2-yl)- | 471 | 8 |
| | 1,2,4-oxadiazol-5-yl]piperidin-1- | | |
| | yl}carbonyl)amino]propyl}pyridinium | | |
| | trifluoroacetate | | |
| 184 | 3-{2,2-dimethyl-3-[({4-[3-(quinolin-2-yl)- | 471 | 8 |
| | 1,2,4-oxadiazol-5-yl]piperidin-1- | | |
| | yl}carbonyl)amino]propyl}pyridinium | | |
| | trifluoroacetate | | |
| 185 | 4-{2,2-dimethyl-3-[({4-[3-(quinolin-2-yl)- | 471 | 8 |
| | 1,2,4-oxadiazol-5-yl]piperidin-1- | | |
| | yl}carbonyl)amino]propyl}pyridinium | | |
| | trifluoroacetate | | |
| 186 | (2R)-1-ethyl-2-{[({4-[3-(quinolin-2-yl)-1,2,4- | 435 | 8 |
| | oxadiazol-5-yl]piperidin-1- | | |
| | yl}carbonyl)amino]methyl}pyrrolidinium | | |
| | trifluoroacetate | | |
| 187 | N-(2-chlorophenyl)-4-[3-(5-chloroquinolin-2- | 468, 470 | 1 |
| | yl)-1,2,4-oxadiazol-5-yl]piperidine-1- | | |
| | carboxamide | | |
| 188 | 5-chloro-2-{5-[1- | 440, 442 | 2 |
| | (cyclohexylcarbamoyl)piperidin-4-yl]-1,2,4- | | |
| | oxadiazol-3-yl}quinolinium trifluoroacetate | | |
| 189 | 4-(1-{[({4-[3-(5-chloroquinolin-2-yl)-1,2,4- | 525, 527 | 8 |
| | oxadiazol-5-yl]piperidin-1- | | |
| | yl}carbonyl)amino]methyl}cyclopentyl)morpholin- | | |
| | 4-ium trifluoroacetate | | |
| 190 | 4-(1-{[({4-[3-(quinazolin-2-yl)-1,2,4- | 492 | 8 |
| | oxadiazol-5-yl]piperidin-1- | | |
| | yl}carbonyl)amino]methyl}cyclopentyl)morpholin- | | |
| | 4-ium trifluoroacetate | | |
| 191 | 2-(5-{1-[(2-oxopiperidin-3- | 421 | 8 |
| | yl)carbamoyl]piperidin-4-yl}-1,2,4-oxadiazol- | | |
| | 3-yl)quinolinium trifluoroacetate | | |
| 192 | 2-(5-{1-[(3,3- | 428 | 8 |
| | difluorocyclopentyl)carbamoyl]piperidin-4- | | |
| | yl}-1,2,4-oxadiazol-3-yl)quinolinium | | |
| | trifluoroacetate | | |
| 193 | 2-(5-{1-[(4,4- | 442 | 8 |
| | difluorocyclohexyl)carbamoyl]piperidin-4- | | |
| | yl}-1,2,4-oxadiazol-3-yl)quinolinium | | |
| | trifluoroacetate | | |
| 194 | 2-{5-[1-(cyclohexylcarbamoyl)piperidin-4- | 420 | 2 |
| | yl]-1,2,4-oxadiazol-3-yl}-3- | | |
| | methylquinolinium trifluoroacetate | | |
| 195 | 2-(5-{1-[methyl(tetrahydro-2H-pyran-4- | 422 | 8 |
| | yl)carbamoyl]piperidin-4-yl}-1,2,4-oxadiazol- | | |
| | 3-yl)quinolinium trifluoroacetate | | |
| 196 | 2-{5-[1-(tetrahydro-2H-pyran-3- | 408 | 8 |
| | ylcarbamoyl)piperidin-4-yl]-1,2,4-oxadiazol- | | |
| | 3-yl}quinolinium trifluoroacetate | | |
| 197 | 2-(5-{1-[methyl(tetrahydrofuran-3- | 408 | 8 |
| | yl)carbamoyl]piperidin-4-yl}-1,2,4-oxadiazol- | | |
| | 3-yl)quinolinium trifluoroacetate | | |
| 198 | tert-butyl 4-[({4-[3-(quinolin-2-yl)-1,2,4- | 507 | 8 |
| | oxadiazol-5-yl]piperidin-1- | | |
| | yl}carbonyl)amino]piperidine-1-carboxylate | | |
| 199 | 2-{5-[1-(cyclohexylcarbamoyl)piperidin-4- | 407 | 2 |
| | yl]-1,2,4-oxadiazol-3-yl}-1,5-naphthyridin-1- | | |
| | ium trifluoroacetate | | |
| 200 | 2-[5-(1-{[(1,1-dioxidotetrahydrothiophen-3- | 456 | 8 |
| | yl)methyl]carbamoyl}piperidin-4-yl)-1,2,4- | | |
| | oxadiazol-3-yl]quinolinium trifluoroacetate | | |
| 201 | 2-(5-{1-[(1,4-dioxan-2- | 424 | 8 |
| | ylmethyl)carbamoyl]piperidin-4-yl}-1,2,4- | | |
| | oxadiazol-3-yl)quinolinium trifluoroacetate | | |
| 202 | 4-(1-{[({4-[3-(3-methylquinolin-2-yl)-1,2,4- | 505 | 8 |
| | oxadiazol-5-yl]piperidin-1- | | |
| | yl}carbonyl)amino]methyl}cyclopentyl)morpholin- | | |
| | 4-ium trifluoroacetate | | |
| 203 | 6-{5-[1-(cyclohexylcarbamoyl)piperidin-4- | 384 | 2 |
| | yl]-1,2,4-oxadiazol-3-yl}-2,3- | | |
| | dimethylpyridinium trifluoroacetate | | |
| 204 | 4-(1-{[({4-[3-(5,6-dimethylpyridinium-2-yl)- | 469 | 8 |
| | 1,2,4-oxadiazol-5-yl]piperidin-1- | | |
| | yl}carbonyl)amino]methyl}cyclopentyl)morpholin- | | |
| | 4-ium bis(trifluoroacetate) | | |
| 205 | 6-(5-{1-[(2- | 412, 414 | 1 |
| | chlorophenyl)carbamoyl]piperidin-4-yl}- | | |
| | 1,2,4-oxadiazol-3-yl)-2,3-dimethylpyridinium | | |
| | trifluoroacetate | | |
| 206 | 4-chloro-2-{5-[1- | 442, 442 | 2 |
| | (cyclohexylcarbamoyl)piperidin-4-yl]-1,2,4- | | |
| | oxadiazol-3-yl}quinolinium trifluoroacetate | | |
| 207 | 1-(propan-2-yl)-2-{[({4-[3-(quinolin-2-yl)- | 449 | 8 |
| | 1,2,4-oxadiazol-5-yl]piperidin-1- | | |
| | yl}carbonyl)amino]methyl}pyrrolidinium | | |
| | trifluoroacetate | | |
| 208 | 2-methyl-1-{[({4-[3-(quinolin-2-yl)-1,2,4- | 483 | 8 |
| | oxadiazol-5-yl]piperidin-1- | | |
| | yl}carbonyl)amino]methyl}-1,2,3,4- | | |
| | tetrahydroisoquinolinium trifluoroacetate | | |
| 209 | 4-(1-{[({4-[3-(6-chloroquinolin-2-yl)-1,2,4- | 525, 527 | 8 |
| | oxadiazol-5-yl]piperidin-1- | | |
| | yl}carbonyl)amino]methyl}cyclopentyl)morpholin- | | |
| | 4-ium trifluoroacetate | | |
| 210 | 2-(5-{1-[(2- | 464, 466 | 1 |
| | chlorophenyl)carbamoyl]piperidin-4-yl}- | | |
| | 1,2,4-oxadiazol-3-yl)-5-methoxyquinolinium | | |
| | trifluoroacetate | | |
| 211 | 2-{5-[1-(cyclohexylcarbamoyl)piperidin-4- | 436 | 2 |
| | yl]-1,2,4-oxadiazol-3-yl}-5- | | |
| | methoxyquinolinium trifluoroacetate | | |
| 212 | 4-(1-{[({4-[3-(5-methoxyquinolin-2-yl)-1,2,4- | 521 | 8 |
| | oxadiazol-5-yl]piperidin-1- | | |
| | yl}carbonyl)amino]methyl}cyclopentyl)morpholin- | | |
| | 4-ium trifluoroacetate | | |
| 213 | 2-(5-{1-[(1-oxidotetrahydro-2H-thiopyran-4- | 440 | 8 |
| | yl)carbamoyl]piperidin-4-yl}-1,2,4-oxadiazol- | | |
| | 3-yl)quinolinium trifluoroacetate | | |
| 214 | 2-(5-{1-[(1-oxidotetrahydro-2H-thiopyran-4- | 440 | 8 |
| | yl)carbamoyl]piperidin-4-yl}-1,2,4-oxadiazol- | | |
| | 3-yl)quinolinium trifluoroacetate | | |
| 215 | 6-cyano-2-{5-[1- | 431 | 2 |
| | (cyclohexylcarbamoyl)piperidin-4-yl]-1,2,4- | | |
| | oxadiazol-3-yl}quinolinium trifluoroacetate | | |
| 216 | N,N,2-trimethyl-1-[({4-[3-(quinolin-2-yl)- | 423 | 8 |
| | 1,2,4-oxadiazol-5-yl]piperidin-1- | | |
| | yl}carbonyl)amino]propan-2-aminium | | |
| | trifluoroacetate | | |
| 217 | 4-{2-[({4-[3-(quinolin-2-yl)-1,2,4-oxadiazol- | 437 | 8 |
| | 5-yl]piperidin-1- | | |
| | yl}carbonyl)amino]ethyl}morpholin-4-ium | | |
| | trifluoroacetate | | |
| 218 | 1-{[({4-[3-(quinolin-2-yl)-1,2,4-oxadiazol-5- | 469 | 8 |
| | yl]piperidin-1-yl}carbonyl)amino]methyl}- | | |
| | 1,2,3,4-tetrahydroisoquinolinium | | |
| | trifluoroacetate | | |
| 219 | N-[2-methyl-3-(pyridin-3-yl)propyl]-4-[3- | 457 | 8 |
| | (quinolin-2-yl)-1,2,4-oxadiazol-5- | | |
| | yl]piperidine-1-carboxamide | | |
| 220 | N-[2-methyl-3-(pyridin-4-yl)propyl]-4-[3- | 457 | 8 |
| | (quinolin-2-yl)-1,2,4-oxadiazol-5- | | |
| | yl]piperidine-1-carboxamide | | |
| 221 | N-[2-methyl-3-(pyridin-2-yl)propyl]-4-[3- | 457 | 8 |
| | (quinolin-2-yl)-1,2,4-oxadiazol-5- | | |
| | yl]piperidine-1-carboxamide | | |
| 222 | tert-butyl 1-{[({4-[3-(quinolin-2-yl)-1,2,4- | 569 | 8 |
| | oxadiazol-5-yl]piperidin-1- | | |
| | yl}carbonyl)amino]methyl}-3,4- | | |
| | dihydroisoquinoline-2(1H)-carboxylate | | |
| 223 | 2-(5-{1-[(4- | 449 | 8 |
| | carbamoylcyclohexyl)carbamoyl]piperidin-4- | | |
| | yl}-1,2,4-oxadiazol-3-yl)quinolinium | | |
| | trifluoroacetate | | |
| 224 | 2-{5-[1-({[1-(morpholin-4-ium-4- | 495 | 8 |
| | yl)cyclopentyl]methyl}carbamoyl)piperidin- | | |
| | 4-yl]-1,2,4-oxadiazol-3-yl}-5,6,7,8- | | |
| | tetrahydroquinolinium bis(trifluoroacetate) | | |
| 225 | N-(1-methyl-1H-benzimidazol-4-yl)-4-[3- | 454 | 1 |
| | (quinolin-2-yl)-1,2,4-oxadiazol-5- | | |
| | yl]piperidine-1-carboxamide | | |
| 226 | 4-[3-(quinolin-2-yl)-1,2,4-oxadiazol-5-yl]-N- | 394 | 8 |
| | (tetrahydrofuran-3-yl)piperidine-1- | | |
| | carboxamide | | |
| 227 | 4-(1-{[({4-[3-(5-cyanoquinolin-2-yl)-1,2,4- | 516 | 8 |
| | oxadiazol-5-yl]piperidin-1- | | |
| | yl}carbonyl)amino]methyl}cyclopentyl)morpholin- | | |
| | 4-ium trifluoroacetate | | |
| 228 | N-cyclohexyl-4-[3-(5,6,7,8- | 410 | 2 |
| | tetrahydroquinolin-2-yl)-1,2,4-oxadiazol-5- | | |
| | yl]piperidine-1-carboxamide | | |
| 229 | 4-(1-{[({4-[3-(quinolin-2-yl)-1,2,4-oxadiazol- | 507 | 8 |
| | 5-yl]piperidin-1- | | |
| | yl}carbonyl)amino]methyl}cyclopentyl)thiomorpholin- | | |
| | 4-ium trifluoroacetate | | |
| 230 | 4-(3-{[({4-[3-(quinolin-2-yl)-1,2,4-oxadiazol- | 509 | 8 |
| | 5-yl]piperidin-1- | | |
| | yl}carbonyl)amino]methyl}tetrahydrothiophen- | | |
| | 3-yl)morpholin-4-ium trifluoroacetate | | |
| 231 | 4-(1-{[({4-[3-(quinolin-2-yl)-1,2,4-oxadiazol- | 539 | 8 |
| | 5-yl]piperidin-1- | | |
| | yl}carbonyl)amino]methyl}cyclopentyl)thiomorpholin- | | |
| | 4-ium 1,1-dioxide trifluoroacetate | | |
| 232 | tert-butyl (1-{[({4-[3-(quinolin-2-yl)-1,2,4- | 521 | 8 |
| | oxadiazol-5-yl]piperidin-1- | | |
| | yl}carbonyl)amino]methyl}cyclopentyl)carbamate | | |
| 233 | 2-(5-{1-[(2- | 440, 442 | 1 |
| | chlorophenyl)carbamoyl]piperidin-4-yl}- | | |
| | 1,2,4-oxadiazol-3-yl)-7,8-dihydro-5H- | | |
| | pyrano[4,3-b]pyridin-1-ium trifluoroacetate | | |
| 234 | 2-{5-[1-(cyclohexylcarbamoyl)piperidin-4- | 412 | 2 |
| | yl]-1,2,4-oxadiazol-3-yl}-7,8-dihydro-5H- | | |
| | pyrano[4,3-b]pyridin-1-ium trifluoroacetate | | |
| 235 | 4-(1-{[({4-[3-(7,8-dihydro-5H-pyrano[4,3- | 497 | 8 |
| | b]pyridin-2-yl)-1,2,4-oxadiazol-5- | | |
| | yl]piperidin-1- | | |
| | yl}carbonyl)amino]methyl}cyclopentyl)morpholin- | | |
| | 4-ium trifluoroacetate | | |
| 236 | 1-(1-{[({4-[3-(5-chloroquinolin-2-yl)-1,2,4- | 437, 439 | 8 |
| | oxadiazol-5-yl]piperidin-1- | | |
| | yl}carbonyl)amino]methyl}cyclohexyl)piperidinium | | |
| | trifluoroacetate | | |
| 237 | 2-(5-{1-[(1-methyl-2-oxopiperidin-3- | 435 | 8 |
| | yl)carbamoyl]piperidin-4-yl}-1,2,4-oxadiazol- | | |
| | 3-yl)quinolinium trifluoroacetate | | |
| 238 | N-{[1-(piperidin-1- | 517 | 8 |
| | ylcarbonyl)cyclopentyl]methyl}-4-[3- | | |
| | (quinolin-2-yl)-1,2,4-oxadiazol-5- | | |
| | yl]piperidine-1-carboxamide | | |
| 239 | 8-chloro-2-{5-[1- | 440, 442 | 2 |
| | (cyclohexylcarbamoyl)piperidin-4-yl]-1,2,4- | | |
| | oxadiazol-3-yl}quinolinium trifluoroacetate | | |
| 240 | 1-(4,4-difluoro-1-{[({4-[3-(quinolin-2-yl)- | 539 | 8 |
| | 1,2,4-oxadiazol-5-yl]piperidin-1- | | |
| | yl}carbonyl)amino]methyl}cyclohexyl)piperidinium | | |
| | trifluoroacetate | | |
| 241 | 1-(2-{[({4-[3-(quinolin-2-yl)-1,2,4-oxadiazol- | 537 | 8 |
| | 5-yl]piperidin-1-yl}carbonyl)amino]methyl}- | | |
| | 2,3-dihydro-1H-inden-2-yl)piperidinium | | |
| | trifluoroacetate | | |
| 242 | 4-fluoro-1-(1-{[({4-[3-(quinolin-2-yl)-1,2,4- | 507 | 8 |
| | oxadiazol-5-yl]piperidin-1- | | |
| | yl}carbonyl)amino]methyl}cyclopentyl)piperidinium | | |
| | trifluoroacetate | | |
| 243 | (3R,4S)-3,4-difluoro-1-(1-{[({4-[3-(quinolin- | 511 | 8 |
| | 2-yl)-1,2,4-oxadiazol-5-yl]piperidin-1- | | |
| | yl}carbonyl)amino]methyl}cyclopentyl)pyrrolidinium | | |
| | trifluoroacetate | | |
| 244 | 4,4-difluoro-1-(1-{[({4-[3-(quinolin-2-yl)- | 525 | 8 |
| | 1,2,4-oxadiazol-5-yl]piperidin-1- | | |
| | yl}carbonyl)amino]methyl}cyclopentyl)piperidinium | | |
| | trifluoroacetate | | |
| 245 | 4-(1-{[({4-[3-(7-chloroquinolin-2-yl)-1,2,4- | 525, 527 | 8 |
| | oxadiazol-5-yl]piperidin-1- | | |
| | yl}carbonyl)amino]methyl}cyclopentyl)morpholin- | | |
| | 4-ium trifluoroacetate | | |
| 246 | 2-{5-[1-({[1- | 463 | 8 |
| | (acetylamino)cyclopentyl]methyl}carbamoyl)piperidin- | | |
| | 4-yl]-1,2,4-oxadiazol-3- | | |
| | yl}quinolinium trifluoroacetate | | |
| 247 | 2-{5-[1-({[1-(pyrrolidinium-1- | 490 | 8 |
| | ylmethyl)cyclopentyl]methyl}carbamoyl)piperidin- | | |
| | 4-yl]-1,2,4-oxadiazol-3-yl}quinolinium | | |
| | bis(trifluoroacetate) | | |
| 248 | N,N-dimethyl-1-{[({4-[3-(quinolin-2-yl)- | 449 | 8 |
| | 1,2,4-oxadiazol-5-yl]piperidin-1- | | |
| | yl}carbonyl)amino]methyl}cyclopentanaminium | | |
| | trifluoroacetate | | |
| 249 | N,N-diethyl-1-{[({4-[3-(quinolin-2-yl)-1,2,4- | 477 | 8 |
| | oxadiazol-5-yl]piperidin-1- | | |
| | yl}carbonyl)amino]methyl}cyclopentanaminium | | |
| | trifluoroacetate | | |
| 250 | 1-(1-{[({4-[3-(quinolin-2-yl)-1,2,4-oxadiazol- | 475 | 8 |
| | 5-yl]piperidin-1- | | |
| | yl}carbonyl)amino]methyl}cyclopentyl)pyrrolidinium | | |
| | trifluoroacetate | | |
| 251 | N-ethyl-1-{[({4-[3-(quinolin-2-yl)-1,2,4- | 449 | 8 |
| | oxadiazol-5-yl]piperidin-1- | | |
| | yl}carbonyl)amino]methyl}cyclopentanaminium | | |
| | trifluoroacetate | | |
| 252 | 1-(1-{[({4-[3-(5-chloroquinolin-2-yl)-1,2,4- | 523, 525 | 8 |
| | oxadiazol-5-yl]piperidin-1- | | |
| | yl}carbonyl)amino]methyl}cyclopentyl)piperidinium | | |
| | trifluoroacetate | | |
| 253 | 6-fluoro-2-{5-[1-({[1-(piperidinium-1- | 507 | 8 |
| | yl)cyclopentyl]methyl}carbamoyl)piperidin- | | |
| | 4-yl]-1,2,4-oxadiazol-3-yl}quinolinium | | |
| | bis(trifluoroacetate) | | |
| 254 | 2-(5-{1-[(4- | 425 | 1 |
| | cyanophenyl)carbamoyl]piperidin-4-yl}- | | |
| | 1,2,4-oxadiazol-3-yl)quinolinium | | |
| | trifluoroacetate | | |
| 255 | 4-(1-oxido-3-{[({4-[3-(quinolin-2-yl)-1,2,4- | 525 | 8 |
| | oxadiazol-5-yl]piperidin-1- | | |
| | yl}carbonyl)amino]methyl}tetrahydrothiophen- | | |
| | 3-yl)morpholin-4-ium trifluoroacetate | | |
| 256 | 6-chloro-2-{5-[1-({[1-(piperidinium-1- | 523, 525 | 8 |
| | yl)cyclopentyl]methyl}carbamoyl)piperidin- | | |
| | 4-yl]-1,2,4-oxadiazol-3-yl}quinolinium | | |
| | bis(trifluoroacetate) | | |
| 257 | 4-[3-(quinolin-2-yl)-1,2,4-oxadiazol1-5-yl]-N- | 469 | 8 |
| | (1,2,3,4-tetrahydroquinolin-2- | | |
| | ylmethyl)piperidine-1-carboxamide | | |
| 258 | 2-({4-[3-(quinolin-2-yl)-1,2,4-oxadiazol-5- | 447 | 8 |
| | yl]piperidin-1-yl}carbonyl)octahydro-1H- | | |
| | pyrrolo[1,2-a][1,4]diazepin-6-ium | | |
| | trifluoroacetate | | |
| 259 | (8aR)-2-({4-[3-(quinolin-2-yl)-1,2,4- | 433 | 8 |
| | oxadiazol-5-yl]piperidin-1- | | |
| | yl}carbonyl)octahydropyrrolo[1,2-a]pyrazin- | | |
| | 5-ium trifluoroacetate | | |
| 260 | 1-ethyl-2-{[({4-[3-(quinolin-2-yl)-1,2,4- | 449 | 8 |
| | oxadiazol-5-yl]piperidin-1- | | |
| | yl}carbonyl)amino]methyl}piperidinium | | |
| | trifluoroacetate | | |
| 261 | N,N-dimethyl-1-[({4-[3-(quinolin-2-yl)-1,2,4- | 409 | 8 |
| | oxadiazol-5-yl]piperidin-1- | | |
| | yl}carbonyl)amino]propan-2-aminium | | |
| | trifluoroacetate | | |
| 262 | (3R)-4-ethyl-3-{[({4-[3-(quinolin-2-yl)-1,2,4- | 451 | 8 |
| | oxadiazol-5-yl]piperidin-1- | | |
| | yl}carbonyl)amino]methyl}morpholin-4-ium | | |
| | trifluoroacetate | | |
| 263 | 4-ethyl-3-{[({4-[3-(quinolin-2-yl)-1,2,4- | 451 | 8 |
| | oxadiazol-5-yl]piperidin-1- | | |
| | yl}carbonyl)amino]methyl}morpholin-4-ium | | |
| | trifluoroacetate | | |
| 264 | (3R)-3-{[({4-[3-(quinolin-2-yl)-1,2,4- | 423 | 8 |
| | oxadiazol-5-yl]piperidin-1- | | |
| | yl}carbonyl)amino]methyl}morpholin-4-ium | | |
| | trifluoroacetate | | |
| 265 | (3S)-3-{[({4-[3-(quinolin-2-yl)-1,2,4- | 423 | 8 |
| | oxadiazol-5-yl]piperidin-1- | | |
| | yl}carbonyl)amino]methyl}morpholin-4-ium | | |
| | trifluoroacetate | | |
| 266 | (3S)-4-ethyl-3-{[({4-[3-(quinolin-2-yl)-1,2,4- | 451 | 8 |
| | oxadiazol-5-yl]piperidin-1- | | |
| | yl}carbonyl)amino]methyl}morpholin-4-ium | | |
| | trifluoroacetate | | |
| 267 | 1-{[({4-[3-(quinolin-2-yl)-1,2,4-oxadiazol-5- | 421 | 8 |
| | yl]piperidin-1- | | |
| | yl}carbonyl)amino]methyl}cyclopentanaminium | | |
| | trifluoroacetate | | |
| 268 | 1-ethyl-2-{[({4-[3-(quinolin-2-yl)-1,2,4- | 435 | 8 |
| | oxadiazol-5-yl]piperidin-1- | | |
| | yl}carbonyl)amino]methyl}pyrrolidinium | | |
| | trifluoroacetate | | |
| 269 | (2S)-1-ethyl-2-{[({4-[3-(quinolin-2-yl)-1,2,4- | 435 | 8 |
| | oxadiazol-5-yl]piperidin-1- | | |
| | yl}carbonyl)amino]methyl}pyrrolidinium | | |
| | trifluoroacetate | | |
| 270 | 2-[5-(1-{[(4-phenyltetrahydro-2H-pyran-4- | 498 | 8 |
| | yl)methyl]carbamoyl}piperidin-4-yl)-1,2,4- | | |
| | oxadiazol-3-yl]quinolinium trifluoroacetate | | |
| 271 | 2-[5-(1-{[(1- | 496 | 8 |
| | phenylcyclohexyl)methyl]carbamoyl}piperidin- | | |
| | 4-yl)-1,2,4-oxadiazol-3-yl]quinolinium | | |
| | trifluoroacetate | | |
| 272 | 2-[5-(1-{[(1- | 482 | 8 |
| | phenylcyclopentyl)methyl]carbamoyl}piperidin- | | |
| | 4-yl)-1,2,4-oxadiazol-3-yl]quinolinium | | |
| | trifluoroacetate | | |
| 273 | 2-(5-{1-[(3,3,3-trifluoro-2- | 434 | 8 |
| | methylpropyl)carbamoyl]piperidin-4-yl}- | | |
| | 1,2,4-oxadiazol-3-yl)quinolinium | | |
| | trifluoroacetate | | |
| 274 | 2-(5-{1-[(spiro[2.5]oct-1- | 446 | 8 |
| | ylmethyl)carbamoyl]piperidin-4-yl}-1,2,4- | | |
| | oxadiazol-3-yl)quinolinium trifluoroacetate | | |
| 275 | 2-{5-[1-({[(1R,2R)-2- | 496 | 8 |
| | phenylcyclohexyl]methyl}carbamoyl)piperidin- | | |
| | 4-yl]-1,2,4-oxadiazol-3-yl}quinolinium | | |
| | trifluoroacetate | | |
| 276 | 2-(5-{1-[(1-methyl-6-oxopiperidin-3- | 435 | 8 |
| | yl)carbamoyl]piperidin-4-yl}-1,2,4-oxadiazol- | | |
| | 3-yl)quinolinium trifluoroacetate | | |
| 277 | N-(propan-2-yl)-1-{[({4-[3-(quinolin-2-yl)- | 463 | 8 |
| | 1,2,4-oxadiazol-5-yl]piperidin-1- | | |
| | yl}carbonyl)amino]methyl}cyclopentanaminium | | |
| | trifluoroacetate | | |
| 278 | N,N-dimethyl-4-{[({4-[3-(quinolin-2-yl)- | 465 | 8 |
| | 1,2,4-oxadiazol-5-yl]piperidin-1- | | |
| | yl}carbonyl)amino]methyl}tetrahydro-2H- | | |
| | pyran-4-aminium trifluoroacetate | | |
| 279 | 1-ethyl-2-{[({4-[3-(quinolin-2-yl)-1,2,4- | 497 | 8 |
| | oxadiazol-5-yl]piperidin-1- | | |
| | yl}carbonyl)amino]methyl}-1,2,3,4- | | |
| | tetrahydroquinolinium trifluoroacetate | | |
| 280 | 1-(3-{[({4-[3-(quinolin-2-yl)-1,2,4-oxadiazol- | 491 | 8 |
| | 5-yl]piperidin-1- | | |
| | yl}carbonyl)amino]methyl}tetrahydrofuran-3- | | |
| | yl)piperidinium trifluoroacetate | | |
| 281 | 4-methoxy-1-(1-{[({4-[3-(quinolin-2-yl)- | 519 | 8 |
| | 1,2,4-oxadiazol-5-yl]piperidin-1- | | |
| | yl}carbonyl)amino]methyl}cyclopentyl)piperidinium | | |
| | trifluoroacetate | | |
| 282 | N-(1-{[({4-[3-(quinolin-2-yl)-1,2,4- | 503 | 8 |
| | oxadiazol-5-yl]piperidin-1- | | |
| | yl}carbonyl)amino]methyl}cyclopentyl)cyclo | | |
| | hexanaminium trifluoroacetate | | |
| 283 | 2-[5-(1-{[2-oxo-2-(piperidin-1- | 449 | 8 |
| | yl)ethyl]carbamoyl}piperidin-4-yl)-1,2,4- | | |
| | oxadiazol-3-yl]quinolinium trifluoroacetate | | |
| 284 | 1-(1-{[({4-[3-(5-fluoroquinolin-2-yl)-1,2,4- | 507 | 8 |
| | oxadiazol-5-yl]piperidin-1- | | |
| | yl}carbonyl)amino]methyl}cyclopentyl)piperidinium | | |
| | trifluoroacetate | | |
| 285 | 1-(1-{[({4-[3-(7-fluoroquinolin-2-yl)-1,2,4- | 507 | 8 |
| | oxadiazol-5-yl]piperidin-1- | | |
| | yl}carbonyl)amino]methyl}cyclopentyl)piperidinium | | |
| | trifluoroacetate | | |
| 286 | 1-(1-{[({4-[3-(3-fluoroquinolin-2-yl)-1,2,4- | 507 | 8 |
| | oxadiazol-5-yl]piperidin-1- | | |
| | yl}carbonyl)amino]methyl}cyclopentyl)piperidinium | | |
| | trifluoroacetate | | |
| 287 | 1-(1-{[({4-[3-(8-fluoroquinolin-2-yl)-1,2,4- | 507 | 8 |
| | oxadiazol-5-yl]piperidin-1- | | |
| | yl}carbonyl)amino]methyl}cyclopentyl)piperidinium | | |
| | trifluoroacetate | | |
| 288 | 2-[5-(1-{[1-(piperidinium-1- | 489 | 8 |
| | ylmethyl)cyclopentyl]carbamoyl}piperidin-4- | | |
| | yl)-1,2,4-oxadiazol-3-yl]quinolinium | | |
| | bis(trifluoroacetate) | | |
| 289 | 2-{5-[1-(cyclohexylcarbamoyl)piperidin-4- | 411 | 2 |
| | yl]-1,2,4-oxadiazol-3-yl}-5,6,7,8-tetrahydro- | | |
| | 1,6-naphthyridin-6-ium trifluoroacetate | | |
| 290 | 4-[3-(6,6-dioxido-7,8-dihydro-5H- | 546 | 8 |
| | thiopyrano[4,3-b]pyridin-2-yl)-1,2,4- | | |
| | oxadiazol-5-yl]-N-{[1-(morpholin-4- | | |
| | yl)cyclopentyl]methyl}piperidine-1- | | |
| | carboxamide | | |

TABLE 6
Benzimidazole and benzothiazole ureidopiperidineoxadiazoles

| | | Molecular Ion | Procedure of |
|---|---|---|---|
| No. | Compound Name | [M + H]⁺ | Example |
| 291 | 2-{5-[1-({[1-(piperidinium-1- | 495 | 10 |
| | yl)cyclopentyl]methyl}carbamoyl)piperidin- | | |
| | 4-yl]-1,2,4-oxadiazol-3-yl}-1,3-benzothiazol- | | |
| | 3-ium bis(trifluoroacetate) | | |
| 292 | N-cyclohexyl-4-{3-[1-(cyclopropylmethyl)- | 449 | 9 |
| | 1H-benzimidazol-2-yl]-1,2,4-oxadiazol-5- | | |
| | yl}piperidine-1-carboxamide | | |
| 293 | N-(2-chlorophenyl)-4-[3-(1-methyl-1H- | 437, 439 | 9 |
| | benzimidazol-2-yl)-1,2,4-oxadiazol-5- | | |
| | yl]piperidine-1-carboxamide | | |
| 294 | N-cyclohexyl-4-[3-(1-ethyl-1H-benzimidazol- | 423 | 9 |
| | 2-yl)-1,2,4-oxadiazol-5-yl]piperidine-1- | | |
| | carboxamide | | |
| 295 | N-cyclohexyl-4-{3-[1-(propan-2-yl)-1H- | 437 | 9 |
| | benzimidazol-2-yl]-1,2,4-oxadiazol-5- | | |
| | yl}piperidine-1-carboxamide | | |
| 296 | 4-(1-{[({4-[3-(1-benzofuran-2-yl)-1,2,4- | 480 | 8 |
| | oxadiazol-5-yl]piperidin-1- | | |
| | yl}carbonyl)amino]methyl}cyclopentyl)morpholin- | | |
| | 4-ium trifluoroacetate | | |
| 297 | 4-[3-(1-methyl-1H-benzimidazol-2-yl)-1,2,4- | 492 | 10 |
| | oxadiazol-5-yl]-N-{[1-(piperidin-1- | | |
| | yl)cyclopentyl]methyl}piperidine-1- | | |
| | carboxamide | | |

TABLE 7
Ureidopiperazineoxadiazoles

| | | Molecular Ion | Procedure of |
|---|---|---|---|
| No. | Compound Name | [M + H]⁺ | Example |
| 298 | 2-{5-[4-({[1-(morpholin-4-ium-4- | 492 | 91 |
| | yl)cyclopentyl]methyl}carbamoyl)piperazin-1- | | |
| | yl]-1,2,4-oxadiazol-3-yl}quinolinium | | |
| | bis(trifluoroacetate) | | |
| 299 | N-{[1-(piperidin-1-yl)cyclopentyl]methyl}-4-[3- | 490 | 91 |
| | (quinolin-2-yl)-1,2,4-oxadiazol-5-yl]piperazine- | | |
| | 1-carboxamide | | |
| 300 | 2-(5-{4-[(2-chlorophenyl)carbamoyl]piperazin- | 435, 437 | 11 |
| | 1-yl}-1,2,4-oxadiazol-3-yl)quinolinium | | |
| | trifluoroacetate | | |
| 301 | 2-(5-{4-[(1- | 421 | 91 |
| | methylcyclohexyl)carbamoyl]piperazin-1-yl}- | | |
| | 1,2,4-oxadiazol-3-yl)quinolinium | | |
| | trifluoroacetate | | |
| 302 | 2-(5-{4-[methyl(tetrahydro-2H-pyran-4- | 423 | 91 |
| | yl)carbamoyl]piperazin-1-yl}-1,2,4-oxadiazol-3- | | |
| | yl)quinolinium trifluoroacetate | | |
| 303 | 2-{5-[4-(tetrahydro-2H-thiopyran-4- | 425 | 91 |
| | ylcarbamoyl)piperazin-1-yl]-1,2,4-oxadiazol-3- | | |
| | yl}quinolinium trifluoroacetate | | |
| 304 | 2-(5-{4-[(1,1-dioxidotetrahydro-2H-thiopyran-4- | 457 | 91 |
| | yl)carbamoyl]piperazin-1-yl}-1,2,4-oxadiazol-3- | | |
| | yl)quinolinium trifluoroacetate | | |
| 305 | 2-[5-(4-{[2-(propan-2- | 443 | 11 |
| | yl)phenyl]carbamoyl}piperazin-1-yl)-1,2,4- | | |
| | oxadiazol-3-yl]quinolinium trifluoroacetate | | |
| 306 | 2-(5-{4-[(3,3- | 429 | 91 |
| | difluorocyclopentyl)carbamoyl]piperazin-1-yl}- | | |
| | 1,2,4-oxadiazol-3-yl)quinolinium | | |
| | trifluoroacetate | | |
| 307 | 4-methyl-1-(1-{[({4-[3-(quinolin-2-yl)-1,2,4- | 519 | 91 |
| | oxadiazol-5-yl]piperazin-1- | | |
| | yl}carbonyl)amino]methyl}cyclohexyl)piperazin- | | |
| | 1-ium trifluoroacetate | | |
| 308 | 1-(1-{[({4-[3-(quinolin-2-yl)-1,2,4-oxadiazol-5- | 504 | 91 |
| | yl]piperazin-1- | | |
| | yl}carbonyl)amino]methyl}cyclohexyl)piperidinium | | |
| | trifluoroacetate | | |
| 309 | 2-(5-{4-[(2,6- | 496, 471 | 11 |
| | dichlorophenyl)carbamoyl]piperazin-1-yl}- | | |
| | 1,2,4-oxadiazol-3-yl)quinolinium | | |
| | trifluoroacetate | | |
| 310 | 2-(5-{4-[(2,3- | 469, 471 | 11 |
| | dichlorophenyl)carbamoyl]piperazin-1-yl}- | | |
| | 1,2,4-oxadiazol-3-yl)quinolinium | | |
| | trifluoroacetate | | |
| 311 | 2-(5-{4- | 393 | 91 |
| | [(cyclobutylmethyl)carbamoyl]piperazin-1-yl}- | | |
| | 1,2,4-oxadiazol-3-yl)quinolinium | | |
| | trifluoroacetate | | |
| 312 | 2-[5-(4-{[2-chloro-4- | 513, 515 | 11 |
| | (methylsulfonyl)phenyl]carbamoyl}piperazin-1- | | |
| | yl)-1,2,4-oxadiazol-3-yl]quinolinium | | |
| | trifluoroacetate | | |
| 313 | 2-{5-[4-({[1-(propan-2-yl)pyrrolidinium-2- | 450 | 91 |
| | yl]methyl}carbamoyl)piperazin-1-yl]-1,2,4- | | |
| | oxadiazol-3-yl}quinolinium bis(trifluoroacetate) | | |
| 314 | 2-{5-[4-(cyclohexylcarbamoyl)piperazin-1-yl]- | 425 | 11 |
| | 1,2,4-oxadiazol-3-yl}-5-fluoroquinolinium | | |
| | trifluoroacetate | | |
| 315 | 2-{5-[4-(bicyclo[1.1.1]pent-1- | 391 | 91 |
| | ylcarbamoyl)piperazin-1-yl]-1,2,4-oxadiazol-3- | | |
| | yl}quinolinium trifluoroacetate | | |
| 316 | 2-(5-{4-[(1- | 393 | 91 |
| | methylcyclobutyl)carbamoyl]piperazin-1-yl}- | | |
| | 1,2,4-oxadiazol-3-yl)quinolinium | | |
| | trifluoroacetate | | |
| 317 | 2-(5-{4- | 421 | 91 |
| | [cyclohexyl(methyl)carbamoyl]piperazin-1-yl}- | | |
| | 1,2,4-oxadiazol-3-yl)quinolinium | | |
| | trifluoroacetate | | |
| 318 | 5-chloro-2-{5-[4- | 441, 443 | 11 |
| | (cyclohexylcarbamoyl)piperazin-1-yl]-1,2,4- | | |
| | oxadiazol-3-yl}quinolinium trifluoroacetate | | |
| 319 | 2-{5-[4-(cyclohexylcarbamoyl)piperazin-1-yl]- | 425 | 11 |
| | 1,2,4-oxadiazol-3-yl}-8-fluoroquinolinium | | |
| | trifluoroacetate | | |
| 320 | 2-(5-{4-[(1- | 407 | 91 |
| | methylcyclopentyl)carbamoyl]piperazin-1-yl}- | | |
| | 1,2,4-oxadiazol-3-yl)quinolinium | | |
| | trifluoroacetate | | |
| 321 | 2-(5-{4-[(3-methyl-1,1- | 457 | 91 |
| | dioxidotetrahydrothiophen-3- | | |
| | yl)carbamoyl]piperazin-1-yl}-1,2,4-oxadiazol-3- | | |
| | yl)quinolinium trifluoroacetate | | |
| 322 | 6-chloro-2-{5-[4- | 441, 443 | 11 |
| | (cyclohexylcarbamoyl)piperazin-1-yl]-1,2,4- | | |
| | oxadiazol-3-yl}quinolinium trifluoroacetate | | |
| 323 | 4-[3-(5-chloroquinolin-2-yl)-1,2,4-oxadiazol-5- | 477, 479 | 91 |
| | yl]-N-(4,4-difluorocyclohexyl)piperazine-1- | | |
| | carboxamide | | |
| 324 | 5-chloro-2-(5-{4-[(1- | 455 | 91 |
| | methylcyclohexyl)carbamoyl]piperazin-1-yl}- | | |
| | 1,2,4-oxadiazol-3-yl)quinolinium | | |
| | trifluoroacetate | | |
| 325 | 5-chloro-2-(5-{4-[methyl(tetrahydro-2H-pyran- | 457, 459 | 91 |
| | 4-yl)carbamoyl]piperazin-1-yl}-1,2,4-oxadiazol- | | |
| | 3-yl)quinolinium trifluoroacetate | | |
| 326 | 2-(5-{4-[(4,4- | 461 | 91 |
| | difluorocyclohexyl)carbamoyl]piperazin-1-yl}- | | |
| | 1,2,4-oxadiazol-3-yl)-5-fluoroquinolinium | | |
| | trifluoroacetate | | |
| 327 | 5-fluoro-2-(5-{4-[(1- | 439 | 91 |
| | methylcyclohexyl)carbamoyl]piperazin-1-yl}- | | |
| | 1,2,4-oxadiazol-3-yl)quinolinium | | |
| | trifluoroacetate | | |
| 328 | 5-fluoro-2-(5-{4-[methyl(tetrahydro-2H-pyran- | 441 | 91 |
| | 4-yl)carbamoyl]piperazin-1-yl}-1,2,4-oxadiazol- | | |
| | 3-yl)quinolinium trifluoroacetate | | |
| 329 | 2-{5-[4-(cyclohexylcarbamoyl)piperazin-1-yl]- | 425 | 11 |
| | 1,2,4-oxadiazol-3-yl}-3-fluoroquinolinium | | |
| | trifluoroacetate | | |
| 330 | 5-chloro-2-(5-{4- | 455, 457 | 91 |
| | [cyclohexyl(methyl)carbamoyl]piperazin-1-yl}- | | |
| | 1,2,4-oxadiazol-3-yl)quinolinium | | |
| | trifluoroacetate | | |
| 331 | 5-chloro-2-(5-{4-[(3,3- | 463, 465 | 91 |
| | difluorocyclopentyl)carbamoyl]piperazin-1-yl}- | | |
| | 1,2,4-oxadiazol-3-yl)quinolinium | | |
| | trifluoroacetate | | |
| 332 | 2-(5-{4- | 439 | 91 |
| | [cyclohexyl(methyl)carbamoyl]piperazin-1-yl}- | | |
| | 1,2,4-oxadiazol-3-yl)-5-fluoroquinolinium | | |
| | trifluoroacetate | | |
| 333 | 2-(5-{4-[(3,3- | 447 | 91 |
| | difluorocyclopentyl)carbamoyl]piperazin-1-yl}- | | |
| | 1,2,4-oxadiazol-3-yl)-5-fluoroquinolinium | | |
| | trifluoroacetate | | |
| 334 | 5-chloro-2-(5-{4-[(4,4- | 491, 493 | 91 |
| | difluorocyclohexyl)(methyl)carbamoyl]piperazin- | | |
| | 1-yl}-1,2,4-oxadiazol-3-yl)quinolinium | | |
| | trifluoroacetate | | |
| 335 | 2-(5-{4-[(4,4- | 474 | 91 |
| | difluorocyclohexyl)(methyl)carbamoyl]piperazin- | | |
| | 1-yl}-1,2,4-oxadiazol-3-yl)-5- | | |
| | fluoroquinolinium trifluoroacetate | | |
| 336 | 6-chloro-2-(5-{4-[methyl(tetrahydro-2H-pyran- | 457, 459 | 91 |
| | 4-yl)carbamoyl]piperazin-1-yl}-1,2,4-oxadiazol- | | |
| | 3-yl)quinolinium trifluoroacetate | | |
| 337 | 6-chloro-2-(5-{4-[(4,4- | 491, 493 | 91 |
| | difluorocyclohexyl)(methyl)carbamoyl]piperazin- | | |
| | 1-yl}-1,2,4-oxadiazol-3-yl)quinolinium | | |
| | trifluoroacetate | | |
| 338 | 6-chloro-2-(5-{4-[(3,3- | 463, 465 | 91 |
| | difluorocyclopentyl)carbamoyl]piperazin-1-yl}- | | |
| | 1,2,4-oxadiazol-3-yl)quinolinium | | |
| | trifluoroacetate | | |
| 339 | 2-{5-[4-(cyclohexylcarbamoyl)piperazin-1-yl]- | 425 | 11 |
| | 1,2,4-oxadiazol-3-yl}-6-fluoroquinolinium | | |
| | trifluoroacetate | | |
| 340 | 2-(5-{4-[(4,4- | 461 | 91 |
| | difluorocyclohexyl)carbamoyl]piperazin-1-yl}- | | |
| | 1,2,4-oxadiazol-3-yl)-6-fluoroquinolinium | | |
| | trifluoroacetate | | |
| 341 | 6-fluoro-2-(5-{4-[methyl(tetrahydro-2H-pyran- | 441 | 91 |
| | 4-yl)carbamoyl]piperazin-1-yl}-1,2,4-oxadiazol- | | |
| | 3-yl)quinolinium trifluoroacetate | | |
| 342 | 2-(5-{4- | 439 | 91 |
| | [cyclohexyl(methyl)carbamoyl]piperazin-1-yl}- | | |
| | 1,2,4-oxadiazol-3-yl)-6-fluoroquinolinium | | |
| | trifluoroacetate | | |
| 343 | 2-(5-{4-[(4,4- | 474 | 91 |
| | difluorocyclohexyl)(methyl)carbamoyl]piperazin- | | |
| | 1-yl}-1,2,4-oxadiazol-3-yl)-6- | | |
| | fluoroquinolinium trifluoroacetate | | |
| 344 | 2-(5-{4-[(3,3- | 447 | 91 |
| | difluorocyclopentyl)carbamoyl]piperazin-1-yl}- | | |
| | 1,2,4-oxadiazol-3-yl)-6-fluoroquinolinium | | |
| | trifluoroacetate | | |
| 345 | 2-{5-[4-(tetrahydro-2H-pyran-4- | 409 | 91 |
| | ylcarbamoyl)piperazin-1-yl]-1,2,4-oxadiazol-3- | | |
| | yl}quinolinium trifluoroacetate | | |
| 346 | 6-chloro-2-(5-{4- | 455, 457 | 91 |
| | [cyclohexyl(methyl)carbamoyl]piperazin-1-yl}- | | |
| | 1,2,4-oxadiazol-3-yl)quinolinium | | |
| | trifluoroacetate | | |
| 347 | 6-fluoro-2-(5-{4-[(1- | 439 | 91 |
| | methylcyclohexyl)carbamoyl]piperazin-1-yl}- | | |
| | 1,2,4-oxadiazol-3-yl)quinolinium | | |
| | trifluoroacetate | | |
| 348 | 6-chloro-2-(5-{4-[(1- | 455, 457 | 91 |
| | methylcyclohexyl)carbamoyl]piperazin-1-yl}- | | |
| | 1,2,4-oxadiazol-3-yl)quinolinium | | |
| | trifluoroacetate | | |

TABLE 8
Benzimidazole and benzothiazole ureidopiperazineoxadiazoles

| | | Molecular Ion | Procedure of |
|---|---|---|---|
| No. | Compound Name | [M + H]⁺ | Example |
| 349 | 1-methyl-2-{5-[4-({[1-(piperidinium-1- | 493 | 94 |
| | yl)cyclopentyl]methyl}carbamoyl)piperazin- | | |
| | 1-yl]-1,2,4-oxadiazol-3-yl}-1H-benzimidazol-3- | | |
| | ium bis(trifluoroacetate) | | |
| 350 | 1-methyl-2-{5-[4-({[1-(propan-2- | 453 | 94 |
| | yl)pyrrolidinium-2- | | |
| | yl]methyl}carbamoyl)piperazin-1-yl]-1,2,4- | | |
| | oxadiazol-3-yl}-1H-benzimidazol-3-ium | | |
| | bis(trifluoroacetate) | | |
| 351 | 2-(5-{4-[(4,4- | 446 | 94 |
| | difluorocyclohexyl)carbamoyl]piperazin-1- | | |
| | yl}-1,2,4-oxadiazol-3-yl)-1-methyl-1H- | | |
| | benzimidazol-3-ium trifluoroacetate | | |
| 352 | N-cyclohexyl-4-[3-(6-fluoro-1-methyl-1H- | 428 | 96 |
| | benzimidazol-2-yl)-1,2,4-oxadiazol-5- | | |
| | yl]piperazine-1-carboxamide | | |
| 353 | N-cyclohexyl-4-[3-(5-fluoro-1-methyl-1H- | 428 | 97 |
| | benzimidazol-2-yl)-1,2,4-oxadiazol-5- | | |
| | yl]piperazine-1-carboxamide | | |
| 354 | N-methyl-4-[3-(1-methyl-1H-benzimidazol-2- | 426 | 95 |
| | yl)-1,2,4-oxadiazol-5-yl]-N-(tetrahydro-2H- | | |
| | pyran-4-yl)piperazine-1-carboxamide | | |
| 355 | 2-(5-{4-[(4,4- | 464 | 96 |
| | difluorocyclohexyl)carbamoyl]piperazin-1- | | |
| | yl}-1,2,4-oxadiazol-3-yl)-7-fluoro-1-methyl- | | |
| | 1H-benzimidazol-3-ium trifluoroacetate | | |
| 356 | 2-(5-{4-[(4,4- | 464 | 97 |
| | difluorocyclohexyl)carbamoyl]piperazin-1- | | |
| | yl}-1,2,4-oxadiazol-3-yl)-4-fluoro-1-methyl- | | |
| | 1H-benzimidazol-3-ium trifluoroacetate | | |
| 357 | 2-{5-[4-(cyclohexylcarbamoyl)piperazin-1- | 428 | 96 |
| | yl]-1,2,4-oxadiazol-3-yl}-7-fluoro-1-methyl- | | |
| | 1H-benzimidazol-3-ium trifluoroacetate | | |
| 358 | 2-{5-[4-(cyclohexylcarbamoyl)piperazin-1- | 428 | 97 |
| | yl]-1,2,4-oxadiazol-3-yl}-4-fluoro-1-methyl- | | |
| | 1H-benzimidazol-3-ium trifluoroacetate | | |
| 359 | 7-fluoro-1-methyl-2-(5-{4- | 444 | 96 |
| | [methyl(tetrahydro-2H-pyran-4- | | |
| | yl)carbamoyl]piperazin-1-yl}-1,2,4- | | |
| | oxadiazol-3-yl)-1H-benzimidazol-3-ium | | |
| | trifluoroacetate | | |
| 360 | 6-fluoro-1-methyl-2-(5-{4- | 444 | 96 |
| | [methyl(tetrahydro-2H-pyran-4- | | |
| | yl)carbamoyl]piperazin-1-yl}-1,2,4- | | |
| | oxadiazol-3-yl)-1H-benzimidazol-3-ium | | |
| | trifluoroacetate | | |
| 361 | 5-fluoro-1-methyl-2-(5-{4- | 444 | 97 |
| | [methyl(tetrahydro-2H-pyran-4- | | |
| | yl)carbamoyl]piperazin-1-yl}-1,2,4- | | |
| | oxadiazol-3-yl)-1H-benzimidazol-3-ium | | |
| | trifluoroacetate | | |
| 362 | 4-fluoro-1-methyl-2-(5-{4- | 444 | 97 |
| | [methyl(tetrahydro-2H-pyran-4- | | |
| | yl)carbamoyl]piperazin-1-yl}-1,2,4- | | |
| | oxadiazol-3-yl)-1H-benzimidazol-3-ium | | |
| | trifluoroacetate | | |
| 363 | 2-{5-[4-(cyclohexylcarbamoyl)piperazin-1- | 413 | 94 |
| | yl]-1,2,4-oxadiazol-3-yl}-1,3-benzothiazol-3- | | |
| | ium trifluoroacetate | | |
| 364 | 2-(5-{4-[(4,4- | 449 | 91 |
| | difluorocyclohexyl)carbamoyl]piperazin-1- | | |
| | yl}-1,2,4-oxadiazol-3-yl)-1,3-benzothiazol-3- | | |
| | ium trifluoroacetate | | |
| 365 | 2-(5-{4-[methyl(tetrahydro-2H-pyran-4- | 428 | 92 |
| | yl)carbamoyl]piperazin-1-yl}-1,2,4- | | |
| | oxadiazol-3-yl)-1,3-benzothiazol-3-ium | | |
| | trifluoroacetate | | |
| 366 | 2-(5-{4-[(1,1-dioxidotetrahydro-2H- | 460 | 94 |
| | thiopyran-4-yl)carbamoyl]piperazin-1-yl}- | | |
| | 1,2,4-oxadiazol-3-yl)-1-methyl-1H- | | |
| | benzimidazol-3-ium trifluoroacetate | | |

1. A compound of structural formula I: 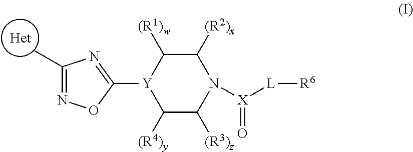
wherein:
each of w, x, y and z is independently 0, 1 or 2;
Y is CH, CR⁵ or N;
L is —(NR⁷)ₐ(O)_{b}(CR⁸R⁹)_{c}(NR⁷)_{d}(C═O)_{f}—;
a is 0 or 1;
b is 0 or 1;
c is 0, 1, 2, 3, 4, 5 or 6;
d is 0 or 1;
f is 0 or 1;
when Y is CH or CR⁵ then each of R¹, R², R³, R⁴ and R⁵ is independently hydroxy, oxo, cyano, halogen, C₁₋₆alkyl, C₂₋₁₀alkenyl, haloC₁₋₆alkyl, hydroxyC₁₋₆alkyl, carboxy, nitro, OR^{a}, CO₂R^{a} or CONR^{a}R^{b};
when Y is N then each of R¹, R², R³ and R⁴ is independently oxo, cyano, C₁₋₆alkyl, C₂₋₁₀alkenyl, haloC₁₋₆alkyl, hydroxyC₁₋₆alkyl, carboxy, CO₂R^{a} or CONR^{a}R^{b};
R⁶ is hydrogen, hydroxy, cyano, halogen, C₁₋₆alkyl, C₂₋₁₀alkenyl, haloC₁₋₆alkyl, hydroxyC₁₋₆alkyl, C₁₋₆alkylcarbonyl, C₁₋₆alkoxy, haloC₁₋₆alkoxy, C₁₋₆alkoxycarbonyl, carboxy, nitro or a ring which is: C₆₋₁₀aryl; C₆₋₁₀aryloxy; C₆₋₁₀arylcarbonyl; C₃₋₁₀cycloalkyl; oxetanyl; azetidinyl; a 5 or 6 membered saturated or partially saturated heterocyclic ring containing one, two or three heteroatoms independently selected from N, O and S; a 5 membered heteroaromatic ring containing 1, 2, 3 or 4 heteroatoms independently selected from N, O and S, not more than one heteroatom of which is O or S; a 6 membered heteroaromatic ring containing one, two or three N atoms; or a 7-15 membered unsaturated, partially saturated or saturated heterocyclic ring containing one, two, three or four heteroatoms independently selected from N, O and S; any of which rings being optionally substituted by one, two or three groups independently selected from (CH₂)ₑ(C═O)_{g}R¹⁰;
e is 0, 1, 2, 3 or 4;
g is 0 or 1;
R⁷ is hydrogen or C₁₋₆alkyl;
each of R⁸ and R⁹ is independently hydrogen, C₁₋₆alkyl, haloC₁₋₆alkyl, NR^{a}R^{b} or a 5 or 6 membered saturated or partially saturated heterocyclic ring containing one, two or three heteroatoms independently selected from N, O and S, which ring is optionally substituted by one, two or three groups independently selected from halogen, C₁₋₆alkyl or haloC₁₋₆alkyl;
Het is pyridin-2-yl or a 7 to 15 membered unsaturated or partially saturated heterocyclic ring containing one, two, three or four heteroatoms independently selected from N, O and S, optionally substituted by one, two or three groups independently selected from R¹¹;
each of R¹⁰ and R¹¹ is independently hydroxy, oxo, oxido, cyano, halogen, C₁₋₆alkyl, C₂₋₁₀alkenyl, haloC₁₋₆alkyl, hydroxyC₁₋₆alkyl, C₁₋₆alkoxyC₁₋₆alkyl, carboxy, nitro, OR^{a}, NR^{a}R^{b}, NR^{a}COR^{b}, NR^{a}S(O)ᵣR^{b}, NR^{a}S(O)ᵣNR^{a}R^{b}, CO₂R^{a}, CONR^{a}R^{b}, S(O)ᵣR^{a}, S(O)ᵣNR^{a}R^{b} or a ring which is: C₃₋₁₀cycloalkyl, C₃₋₁₀cycloalkylC₁₋₆alkyl, C₆₋₁₀aryl, C₆₋₁₀aryloxy, azetidinyl or a 5 or 6 membered saturated or partially saturated heterocyclic ring containing one, two or three heteroatoms independently selected from N, O and S, a 5 membered heteroaromatic ring containing 1, 2, 3 or 4 heteroatoms independently selected from N, O and S, not more than one heteroatom of which is O or S or a 6 membered heteroaromatic ring containing one, two or three N atoms, any of which rings being optionally substituted by one, two or three groups independently selected from hydroxy, oxo, oxido, halogen, C₁₋₆alkyl, haloC₁₋₆alkyl and C₁₋₆alkoxy;
each of R^{a} and R^{b} is independently hydrogen, C₁₋₆alkyl, C₁₋₆alkoxy, C₁₋₆alkylcarbonyl, haloC₁₋₆alkyl, hydroxyC₁₋₆alkyl or C₃₋₁₀cycloalkyl;
r is 0, 1 or 2;
X is C or S═O;
or a pharmaceutically acceptable salt, stereoisomer or tautomer thereof.

2. A compound of claim 1 of formula II: 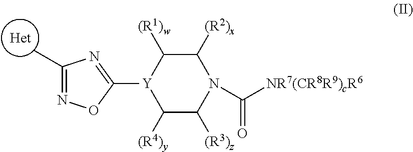
wherein c, w, x, y, z, Y, R¹, R², R³, R⁴, R⁵, R⁶, R⁷, R⁸, R⁹ and Het are as defined in claim 1;
or a pharmaceutically acceptable salt, stereoisomer or tautomer thereof.

3. A compound of claim 1 of formula III: 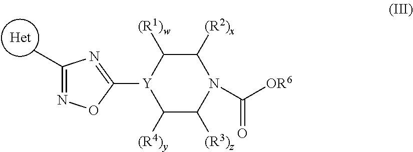
wherein w, x, y, z, Y, R¹, R², R³, R⁴, R⁵, R⁶ and Het are as defined in claim 1;
or a pharmaceutically acceptable salt, stereoisomer or tautomer thereof.

4. A compound of claim 1 wherein Het is a 8 to 10 membered unsaturated or partially saturated heterocyclic ring containing one, two, three or four heteroatoms independently selected from N, O and S, optionally substituted by one, two or three groups independently selected from R¹¹.

5. A compound of claim 1 of formula IV: 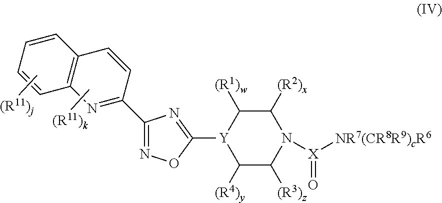
wherein:
the sum of j and k is 0, 1, 2 or 3;
c, w, x, y, z, Y, L, R¹, R², R³, R⁴, R⁵, R⁶, R⁷, R⁸, R⁹, R¹¹ and X are as defined in claim 1;
or a pharmaceutically acceptable salt, stereoisomer or tautomer thereof.

6. A compound of claim 1 of formula V: 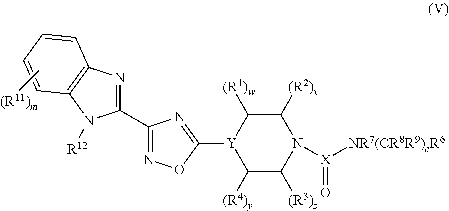
wherein:
m is 0, 1 or 2;
R¹² is hydrogen or R¹¹;
c, w, x, y, z, Y, L, R¹, R², R³, R⁴, R⁵, R⁶, R⁷, R⁸, R⁹, R¹¹ and X are as defined in claim 1;
or a pharmaceutically acceptable salt, stereoisomer or tautomer thereof.

7. A compound of claim 1 wherein R⁶ is C₁₋₆alkyl, such as methyl haloC₁₋₆alkyl, such as trifluoromethyl or a ring which is: phenyl, cyclohexyl, cycloheptyl, tetrahydrothiopyranyl, cyclopropyl, naphthyl, indolyl, dihydroquinolinyl, furyl, pyridinyl, dihydrobenzodioxinyl, benzofuranyl, cyclobutyl, tetrahydropyranyl, quinolinyl, piperidinyl, dihydroindenyl, adamantyl, benzodioxolyl, cyclopentyl, isoxazolyl, azaazoniaspirononyl, triazolyl, pyrazinyl, azaazoniaspirodecyl, dihydropyridinyl, quinoxalinyl, benzoxazolyl, benzothiazolyl, benzodioxinyl, tetrahydrofuranyl, thiazolyl, tetrahydrothiophenyl, benzimidazolyl, pyrrolidinyl, morpholinyl, dioxanyl, tetrahydroquinolinyl, tetrahydroisoquinolinyl, octahydropyrrolodiazepinyl, octahydropyrrolopyrazinyl, spirooctyl or bicyclopentyl; any of which rings being optionally substituted by one, two or three groups independently selected from (CH₂)ₑ(C═O)_{f}R¹⁰.

8. A compound of claim 1 wherein Y is N.

9. A compound of claim 1 wherein Y is CH or CR⁵.

10. A pharmaceutical composition comprising a compound of claim 1, or a pharmaceutically acceptable salt, stereoisomer or tautomer thereof in association with a pharmaceutically acceptable carrier.

11-16. (canceled)

17. A method of treating or preventing cancer, which method comprises administration to a patient in need thereof of an effective amount of a compound of claim 1 or a composition comprising a compound of claim 1.
